SEQUENCE LISTING (111 sequences)

SEQ ID NO: 1 - DNA, 564 bp, Gallus gallus
  source 1..564: mol_type = genomic DNA
gataccggttttccgggtatgcctggtcgtagcggtgatccgggtcgtagcggtaaagatggtctgcctggtagcccgggttttaaaggtgaagttggtcagccaggtagccctggtctggaaggtcatcgtggtgaaccgggtattccaggtattccgggtaatcagggtgcaaaaggtcagaaaggcgaaattggtcctccgggtctgccaggtgccaaaggttctccgggtgaaaccggtctgatgggtcctgaaggtagctttggcctgcctggtgcaccgggtccgaaaggtgacaaaggtgaacctggtctgcagggtaaaccgggtagcagcggtgcaaaaggcgaaccaggtggtccgggtgctccgggtgaaccaggctatccgggtattcctggtactcagggtattaaaggcgataaaggtagccagggtgaaagcggtattcagggtcgtaagggtgaaaaaggccgtcagggtaatccaggcctgcagggcaccgaaggtctgcgtggcgaacagggcgaaaaaggtgagaagggtgacccaggcattcgt

SEQ ID NO: 2 - protein, 188 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIR

SEQ ID NO: 3 - DNA, 564 bp, Gallus gallus
  source 1..564: mol_type = genomic DNA
gatactggtttcccggggatgcctgggcgctcaggtgatccggggcgtagtggaaaagacggtctgccggggtccccgggctttaagggtgaggtgggtcagcccggtagtccaggtttagaaggtcaccgcggagagcccgggattccaggcattcctggcaaccagggtgccaagggacagaaaggcgaaattggtccgcccggcctaccgggcgcgaaaggttctcctggtgaaaccggtctcatgggtccggaaggtagcttcggcctgcccggcgcacctggtccgaagggcgataagggggagcctgggctgcaaggtaaaccgggtagttctggcgccaaaggtgaacccggcggtcccggtgcgccaggggaaccaggttatcctggtattcctggaacccaaggaattaaaggtgacaaaggctcacagggcgaaagtggtatacagggtcgcaagggcgaaaaaggacgtcagggcaatccaggcctgcagggtactgaaggcctgcgtggagaacagggtgagaaaggtgaaaaaggagatcctggtattcgc

SEQ ID NO: 4 - protein, 188 aa, Gallus gallus; identical to SEQ ID NO: 2

SEQ ID NO: 5 - DNA, 633 bp, Gallus gallus
  source 1..633: mol_type = genomic DNA
gatactggtttcccggggatgcctgggcgctcaggtgatccggggcgtagtggaaaagacggtctgccggggtccccgggctttaagggtgaggtgggtcagcccggtagtccaggtttagaaggtcaccgcggagagcccgggattccaggcattcctggcaaccagggtgccaagggacagaaaggcgaaattggtccgcccggcctaccgggcgcgaaaggttctcctggtgaaaccggtctcatgggtccggaaggtagcttcggcctgcccggcgcacctggtccgaagggcgataagggggagcctgggctgcaaggtaaaccgggtagttctggcgccaaaggtgaacccggcggtcccggtgcgccaggggaaccaggttatcctggtattcctggaacccaaggaattaaaggtgacaaaggctcacagggcgaaagtggtatacagggtcgcaagggcgaaaaaggacgtcagggcaatccaggcctgcagggtactgaaggcctgcgtggagaacagggtgagaaaggtgaaaaaggagatcctggtattcgcggcattaacggtcaaaagggtgaaagtgggatacaaggtcttgtcggtccgcccggagttagaggccag

SEQ ID NO: 6 - protein, 211 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQ

SEQ ID NO: 7 - DNA, 596 bp, Acipenser schrenckii
  source 1..596: mol_type = genomic DNA
gtctgcagggtatgcctggtgaacgtggtgcaagcggtattgccggtgcaaaaggtgatcgtggtgatgttggtgaaaaaggtccggaaggtgccagcggtaaagatggtagccgtggtctgaccggtccgattggtccgcctggtccggcaggtccgaatggcgaaaaaggtgaaagcggtccgagcggtcctccgggtgcagcaggtactcgtggtgcaccgggtgatcgcggtgaaaatggtccaccgggtcctgccggttttgcaggtccgccaggtgcagatggtcagcctggtgccaaaggcgaacaaggcgaaggtggtcagaaaggtgatgcaggcgctccgggtccgcagggtccttctggtgcacctggtcctcagggtccgaccggtgtttctggtccgaaaggcgcacgtggtgcccagggtccacctggtgcgaccggttttcctggcgcagcaggtcgtgttggtcctccaggtcctaatggtaatccgggtccaagcggtcctgcaggtagcgcaggcaaagatggtcctaaaggtgtacgcggtgatgctggtcctcctggccgtgccggtgatgccggt

SEQ ID NO: 8 - protein, 199 aa, Acipenser schrenckii
GLQGMPGERGASGIAGAKGDRGDVGEKGPEGASGKDGSRGLTGPIGPPGPAGPNGEKGESGPSGPPGAAGTRGAPGDRGENGPPGPAGFAGPPGADGQPGAKGEQGEGGQKGDAGAPGPQGPSGAPGPQGPTGVSGPKGARGAQGPPGATGFPGAAGRVGPPGPNGNPGPSGPAGSAGKDGPKGVRGDAGPPGRAGDAG

SEQ ID NO: 9 - DNA, 57 bp, unidentified
  source 1..57: mol_type = unassigned DNA
atgaaaaagatttggctggcgctggctggtttagttttagcgtttagcgcatcggcg

SEQ ID NO: 10 - protein, 19 aa, unidentified
MKKIWLALAGLVLAFSASA

SEQ ID NO: 11 - DNA, 60 bp, unidentified
  source 1..60: mol_type = unassigned DNA
atgaaaaaaggtttcatgctgttcaccctcctcgctgcgttctctggtttcgcgcaggct

SEQ ID NO: 12 - protein, 20 aa, unidentified
MKKGFMLFTLLAAFSGFAQA

SEQ ID NO: 13 - DNA, 75 bp, unidentified
  source 1..75: mol_type = unassigned DNA
atgatgatcaccctgcgtaaactgccgctggctgttgctgttgctgctggtgttatgtctgctcaggctatggct

SEQ ID NO: 14 - protein, 25 aa, unidentified
MMITLRKLPLAVAVAAGVMSAQAMA

SEQ ID NO: 15 - DNA, 63 bp, unidentified
  source 1..63: mol_type = unassigned DNA
atgaaaaaaaccgctatcgctatcgctgttgctctggctggtttcgctaccgttgctcaggct

SEQ ID NO: 16 - protein, 21 aa, unidentified
MKKTAIAIAVALAGFATVAQA

SEQ ID NO: 17 - DNA, 63 bp, unidentified
  source 1..63: mol_type = unassigned DNA
atgaaagttaaagttctgtctctgctggttccggctctgctggttgctggtgctgctaacgct

SEQ ID NO: 18 - protein, 21 aa, unidentified
MKVKVLSLLVPALLVAGAANA

SEQ ID NO: 19 - DNA, 78 bp, unidentified
  source 1..78: mol_type = unassigned DNA
atgaaaaaaaacatcctgtctctgtctatggttgctctgtctctgtctctggctctgggttctgtttctgttaccgct

SEQ ID NO: 20 - protein, 26 aa, unidentified
MKKNILSLSMVALSLSLALGSVSVTA

SEQ ID NO: 21 - DNA, 69 bp, unidentified
  source 1..69: mol_type = unassigned DNA
atgctgaacccgaaagttgcttacatggtttggatgacctgcctgggtctgaccctgccgtctcaggct

SEQ ID NO: 22 - protein, 23 aa, unidentified
MLNPKVAYMVWMTCLGLTLPSQA

SEQ ID NO: 23 - DNA, 63 bp, unidentified
  source 1..63: mol_type = unassigned DNA
atgaaacaggctctgcgtgtagcgttcggtttcctgatactgtgggcttctgttctgcacgct

SEQ ID NO: 24 - protein, 21 aa, unidentified
MKQALRVAFGFLILWASVLHA

SEQ ID NO: 25 - DNA, 600 bp, synthetic construct
  source 1..600: mol_type = other DNA
ggtctgcagggtatgccgggtgaacgtggtgccagcggtattgcaggtgccaaaggtgatcgtggtgatgttggtgaaaaaggtccggaaggtgcaagcggtaaagatggtagccgtggtctgaccggtccgattggtccgccgggtccggccggtccgaatggtgaaaaaggtgaaagcggtccgagcggtccgccgggtgcagccggtacccgtggtgcaccgggtgatcgtggtgaaaatggtccgccgggtccggccggttttgcaggtccgccgggtgccgatggtcagccgggtgcaaaaggtgaacagggtgaaggtggtcagaaaggtgatgccggtgcaccgggtccgcagggtccgagcggtgccccgggtccgcagggtccgaccggtgttagcggtccgaaaggtgcacgtggtgcccagggtccgccgggtgcaaccggttttccgggtgccgcaggtcgtgttggtccgccgggtccgaatggtaatccgggtccgagcggtccggcaggtagcgccggtaaagatggtccgaaaggtgttcgtggtgatgcaggtccgccgggtcgtgccggtgatgcaggttaa

SEQ ID NO: 26 - DNA, 600 bp, synthetic construct
  source 1..600: mol_type = other DNA
ggcctgcaaggcatgccaggcgagcgcggcgcgtctggcatcgcgggcgcgaagggcgaccgcggcgacgtgggcgagaagggccctgagggcgcgtccggcaaggacggctctcgcggcctgacaggcccaatcggccctccaggccctgcgggcccaaacggcgagaagggcgagtccggcccttctggcccacctggcgcggcgggcacacgcggcgcgccaggcgaccgcggcgagaacggccctccaggccctgcgggcttcgcgggcccacctggcgcggacggccaaccaggcgcgaagggcgagcaaggcgagggcggccaaaagggcgacgcgggcgcgcctggcccacaaggcccttctggcgcgccaggccctcaaggcccaacaggcgtgtccggccctaagggcgcgcgcggcgcgcaaggcccacctggcgcgacaggcttcccaggcgcggcgggccgcgtgggccctccaggccctaacggcaacccaggcccttctggcccagcgggctccgcgggcaaggacggccctaagggcgtgcgcggcgacgcgggcccacctggccgcgcgggcgacgcgggctga

SEQ ID NO: 27 - DNA, 600 bp, synthetic construct
  source 1..600: mol_type = other DNA
ggtttgcaaggtatgccaggggaacggggtgcgtccgggatagccggggcaaaaggtgatcgaggcgatgtaggagaaaaaggcccagaaggggcgtcaggtaaggacggatctcgcggcttgacgggacctatcgggcctccaggtcccgccggccctaatggggaaaaaggcgagagtgggccgtctggtccgcccggcgccgctggcacacgtggagcgccgggcgatcgtggtgagaacggaccaccgggtcctgctggttttgcgggacctccgggagcagacggccagccgggcgctaaaggtgaacagggtgaaggtggccaaaaaggcgatgcaggcgcaccgggtccgcagggcccttcaggtgcaccgggtccacagggcccaactggcgtttcagggccgaaaggcgcaagaggtgctcagggtccgcccggggcaactgggtttcctggagcggccggccgtgttggacctccggggccgaacggaaaccctggaccgtctggaccagccggttcagcgggtaaggatggtcctaagggtgtaaggggtgacgcaggtccccctggacgtgcaggggatgcggggtag

SEQ ID NO: 28 - DNA, 600 bp, synthetic construct
  source 1..600: mol_type = other DNA
gggttacaaggtatgccgggagaacgtggagcgtcaggaattgctggggccaaaggtgatcgtggtgatgttggcgagaaagggcccgaaggcgcatctggtaaagatggctcacgcgggttaactggaccaatcggaccaccaggccccgctgggcctaatggtgaaaagggtgaaagtggcccttctggacccccaggagccgccggtacacgtggagcgccaggcgatcgtggcgaaaacggaccgcccggacctgcaggttttgcgggaccccctggagcagacggccaaccaggagcaaaaggtgagcaaggtgaaggtggacaaaagggagatgccggagcgccaggcccccaaggcccatcaggagctccaggacctcaaggtccaactggtgtatcagggcctaagggtgcgcgcggcgctcaaggaccgcctggcgcaactggctttccgggagctgctggtcgtgtgggcccgcctggcccaaacggaaatccaggcccttcaggcccggcgggctcagccggaaaagacggtccgaagggagtccgtggagatgcgggaccgccaggacgcgctggcgatgcaggctaa

SEQ ID NO: 29 - DNA, 600 bp, synthetic construct
  source 1..600: mol_type = other DNA
ggtttacagggaatgccaggggaacgcggcgcctcagggattgccggtgctaaaggagatcgtggcgacgtgggtgaaaagggtcccgagggagcatcaggtaaggatggttcccgtggtttgacgggacctattggacctccgggtcctgcaggtccgaacggcgaaaagggggaaagcgggcctagtggtccacccggcgccgcaggtacccgtggtgccccaggcgaccgcggggagaatggaccgcctggccctgccggttttgcgggtcctccaggagccgatgggcagcccggtgcaaaaggagagcagggagagggaggtcaaaagggagatgccggcgccccgggccctcagggaccaagcggtgcgccaggcccccagggtcctacgggtgttagcgggccgaaaggcgcacgcggagcgcagggcccacctggtgcaacaggcttcccaggagctgcggggcgcgtcggacctccgggacccaatggaaacccaggtccgtcagggccggcaggctccgcagggaaagatggtcccaaaggcgtgcgtggagacgcagggccccccggacgcgccggcgatgcgggataa

SEQ ID NO: 30 - DNA, 564 bp, synthetic construct; identical to SEQ ID NO: 3
  source 1..564: mol_type = other DNA

SEQ ID NO: 31 - protein, 957 aa, Gallus gallus
MAQLLRLFQTLLILLLRDYISAEDGETRASCRTAPADLVFILDGSYSVGPENFEIIKSWLVNITRNFDIGPKFIQVGVVQYSDYPVLEIPLGTHESTENLIKEMESIHYLGGNTKTGRAIQFAYDHLFAKSSRFLTKIAVVLTDGKSQDEVKDVAAEARKNKITLFAIGVGSEIEEDELKAIANKPSSTYVFYVEDYIAISRIKEVIKQKLCEESVCPTRIPVAARDEKGFDILVGLGVKKRVKKRIQIPTTNAKAYEVTSRVDLSELTRNVFPEGLPPSYVFVSTQRFKVKKTWDLWRVLSLDKRPQIAVTINGEEKTLSFTTTSLINGTQVITFAAPRVKTLFDEGWHQIRLLVTEDFVTLYIDDQEIETKPLHPVLGIYISGLTQIGKYSGKEETVQFDIQKLRIYCDPEQNNRETVCEIPGFNGECMNGPSDVGSTPAPCICPPGKQGPPGPKGDPGQPGNHGYPGQPGPDGKPGYQGSAGTPGIPGTPGVQGPRGLPGIKGEPGKDGTKGDRGLPGFPGLHGMPAPKGERGPKGDQGVPGIYGKKGSKGEKGDTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQPGDRGPPGPPGSDGKPAREFSEEFIRQVCSDVLRTQLPVILQSGRLQNCNHCQSQSASPGLPGPPGPRGPEGPRGFPGLPGNDGVPGLTGIPGRPGARGTRGLPGKNGAKGNQGIGVPGIQGPPGPPGPEGPPGMSKEGRPGERGQPGKDGDRGSPGMPGPVGPPGICDPSLCFSVIVGRDPFRKGPNY

SEQ ID NO: 32 - protein, 1420 aa, Acipenser schrenckii
MFSFVDSRTVLLLAAIQLCLLAVVKCQDVEVQQPGRKGQKGEPGDITDVVGPRGPGGPMGPPGEQGPRGERGDKGDKGGPGPRGRDGEPGTPGNPGPPGPPGPNGPPGLGGNFAAQMAGGFDEKAGGAQMGVMQGPMGPMGPRGPPGPTGAPGPQGFQGNPGEPGEPGAAGPLGPRGPPGPSGKPGEDGEAGKPGKSGERGSPGPQGARGFPGTPGLPGIKGHRGYPGLDGAKGEAGAAGSKGEAGSSGENGAPGPMGPRGLPGERGRNGPSGAAGARGNDGLPGPAGPPGPVGPAGAPGFPGSPGSKGEAGPTGARGPEGAQGPRGESGTPGSPGPSGASGNPGTDGIPGAKGSAGAPGIAGAPGFPGPRGPPGPQGATGPLGPKGQQGDPGIPGFKGEHGPKGEHGPAGPQGAPGPAGEEGKRGARGEPGAAGPLGPPGERGAPGNRGFPGQDGLAGPKGAPGERGQPGVGGPKGANGDPGRPGEPGLPGARGLTGRPGDAGPQGKGGPSGAAGEDGRPGPPGPQGARGQPGVMGFPGPKGANGEPGKAGEKGLVGPPGLRGLSGKDGETGAAGPPGPSGPAGERGEQGPPGPSGFQGLPGPPGPPGEGGKPGDQGVPGEAGAAGRAGPRGERGFPGERGSPGAQGLQGPRGLPGTPGTDGPKGATGPSGALGAQGPPGLQGMPGERGASGIAGAKGDRGDVGEKGPEGASGKDGSRGLTGPIGPPGPAGPNGEKGESGPSGPPGAAGTRGAPGDRGENGPPGPAGFAGPPGADGQPGAKGEQGEGGQKGDAGAPGPQGPSGAPGPQGPTGVSGPKGARGAQGPPGATGFPGAAGRVGPPGPNGNPGPSGPAGSAGKDGPKGVRGDAGPPGRAGDAGLQGAAGPPGEKGEPGEDGPPGPDGPSGPQGLGGNRGIVGLPGQRGERGFPGLPGPSGEPGKQGAPGGAGDRGPPGPVGPPGLSGPSGEPGREGNPGSDGPPGRDGSAGIKGDRGQTGPAGAPGAPGAPGSPGPVGPTGKQGDRGESGAQGPAGPSGPAGARGMAGPQGPRGDKGEAGETGERGQKGHRGFTGLQGLPGPPGTAGDQGAAGPAGPTGARGPPGPVGPHGKDGSNGQPGPIGPPGPRGRSGEVGPAGPPGNAGPPGPPGPPGPGIDMSAFAGLAAPEKAPDPMRYMRADEASSSLRQHDAEVDATLKSINNQIENIRSPEGSKKNPARTCRDLKLCHPDWKSGDYWIDPNQGCAVDAIKVFCNMESGETCVYPNPASIPRKNWWTSKSADCKHVWFGETMNGGFHFSYGDDSLAPNTASIQMTFLRLLSTEASQNLTYHCKNSIAYMDQSAGNLKKAVLLQGSNDVEIRAEGNSRFTYNVLEDGCTKHTDRWGKTVIEYKSQKTSRLPIVDIAPLDIGGSDQEFGVDIGPVCY

SEQ ID NO: 33 - protein, 213 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQPG

SEQ ID NO: 34 - protein, 199 aa, Acipenser schrenckii; identical to SEQ ID NO: 8

SEQ ID NO: 35 - protein, 10 aa, Gallus gallus
DTGFPGMPGR

SEQ ID NO: 36 - protein, 13 aa, Gallus gallus
DTGFPGMPGRSGD

SEQ ID NO: 37 - protein, 16 aa, Gallus gallus
DTGFPGMPGRSGDPGR

SEQ ID NO: 38 - protein, 19 aa, Gallus gallus
DTGFPGMPGRSGDPGRSGK

SEQ ID NO: 39 - protein, 29 aa, Gallus gallus
DTGFPGMPGRSGDPGRSGKDGLPGSPGFK

SEQ ID NO: 40 - protein, 9 aa, Gallus gallus
TGFPGMPGR

SEQ ID NO: 41 - protein, 17 aa, Gallus gallus
GFPGMPGRSGDPGRSGK

SEQ ID NO: 42 - protein, 15 aa, Gallus gallus
PGMPGRSGDPGRSGK

SEQ ID NO: 43 - protein, 12 aa, Gallus gallus
PGRSGDPGRSGK

SEQ ID NO: 44 - protein, 6 aa, Gallus gallus
SGDPGR

SEQ ID NO: 45 - protein, 19 aa, Gallus gallus
SGDPGRSGKDGLPGSPGFK

SEQ ID NO: 46 - protein, 10 aa, Gallus gallus
DGLPGSPGFK

SEQ ID NO: 47 - protein, 12 aa, Gallus gallus
DGLPGSPGFKGE

SEQ ID NO: 48 - protein, 25 aa, Gallus gallus
DGLPGSPGFKGEVGQPGSPGLEGHR

SEQ ID NO: 49 - protein, 40 aa, Gallus gallus
DGLPGSPGFKGEVGQPGSPGLEGHRGEPGIPGIPGNQGAK

SEQ ID NO: 50 - protein, 43 aa, Gallus gallus
DGLPGSPGFKGEVGQPGSPGLEGHRGEPGIPGIPGNQGAKGQK

SEQ ID NO: 51 - protein, 39 aa, Gallus gallus
GLPGSPGFKGEVGQPGSPGLEGHRGEPGIPGIPGNQGAK

SEQ ID NO: 52 - protein, 22 aa, Gallus gallus
PGSPGFKGEVGQPGSPGLEGHR

SEQ ID NO: 53 - protein, 15 aa, Gallus gallus
GEVGQPGSPGLEGHR

SEQ ID NO: 54 - protein, 30 aa, Gallus gallus
GEVGQPGSPGLEGHRGEPGIPGIPGNQGAK

SEQ ID NO: 55 - protein, 33 aa, Gallus gallus
GEVGQPGSPGLEGHRGEPGIPGIPGNQGAKGQK

SEQ ID NO: 56 - protein, 16 aa, Gallus gallus
RGEPGIPGIPGNQGAK

SEQ ID NO: 57 - protein, 15 aa, Gallus gallus
GEPGIPGIPGNQGAK

SEQ ID NO: 58 - protein, 18 aa, Gallus gallus
GEPGIPGIPGNQGAKGQK

SEQ ID NO: 59 - protein, 30 aa, Gallus gallus
GEPGIPGIPGNQGAKGQKGEIGPPGLPGAK

SEQ ID NO: 60 - protein, 54 aa, Gallus gallus
GEPGIPGIPGNQGAKGQKGEIGPPGLPGAKGSPGETGLMGPEGSFGLPGAPGPK

SEQ ID NO: 61 - protein, 10 aa, Gallus gallus
PGNQGAKGQK

SEQ ID NO: 62 - protein, 22 aa, Gallus gallus
PGNQGAKGQKGEIGPPGLPGAK

SEQ ID NO: 63 - protein, 15 aa, Gallus gallus
GQKGEIGPPGLPGAK

SEQ ID NO: 64 - protein, 13 aa, Gallus gallus
KGEIGPPGLPGAK

SEQ ID NO: 65 - protein, 12 aa, Gallus gallus
GEIGPPGLPGAK

SEQ ID NO: 66 - protein, 54 aa, Gallus gallus
GEIGPPGLPGAKGSPGETGLMGPEGSFGLPGAPGPKGDKGEPGLQGKPGSSGAK

SEQ ID NO: 67 - protein, 10 aa, Gallus gallus
IGPPGLPGAK

SEQ ID NO: 68 - protein, 24 aa, Gallus gallus
GSPGETGLMGPEGSFGLPGAPGPK

SEQ ID NO: 69 - protein, 26 aa, Gallus gallus
GSPGETGLMGPEGSFGLPGAPGPKGD

SEQ ID NO: 70 - protein, 27 aa, Gallus gallus
GSPGETGLMGPEGSFGLPGAPGPKGDK

SEQ ID NO: 71 - protein, 19 aa, Gallus gallus
TGLMGPEGSFGLPGAPGPK

SEQ ID NO: 72 - protein, 18 aa, Gallus gallus
GLMGPEGSFGLPGAPGPK

SEQ ID NO: 73 - protein, 11 aa, Gallus gallus
SFGLPGAPGPK

SEQ ID NO: 74 - protein, 18 aa, Gallus gallus
GDKGEPGLQGKPGSSGAK

SEQ ID NO: 75 - protein, 16 aa, Gallus gallus
KGEPGLQGKPGSSGAK

SEQ ID NO: 76 - protein, 15 aa, Gallus gallus
GEPGLQGKPGSSGAK

SEQ ID NO: 77 - protein, 42 aa, Gallus gallus
GEPGLQGKPGSSGAKGEPGGPGAPGEPGYPGIPGTQGIKGDK

SEQ ID NO: 78 - protein, 24 aa, Gallus gallus
GEPGGPGAPGEPGYPGIPGTQGIK

SEQ ID NO: 79 - protein, 27 aa, Gallus gallus
GEPGGPGAPGEPGYPGIPGTQGIKGDK

SEQ ID NO: 80 - protein, 30 aa, Gallus gallus
PGEPGYPGIPGTQGIKGDKGSQGESGIQGR

SEQ ID NO: 81 - protein, 27 aa, Gallus gallus
PGYPGIPGTQGIKGDKGSQGESGIQGR

SEQ ID NO: 82 - protein, 24 aa, Gallus gallus
PGIPGTQGIKGDKGSQGESGIQGR

SEQ ID NO: 83 - protein, 21 aa, Gallus gallus
PGTQGIKGDKGSQGESGIQGR

SEQ ID NO: 84 - protein, 22 aa, Gallus gallus
PGTQGIKGDKGSQGESGIQGRK

SEQ ID NO: 85 - protein, 14 aa, Gallus gallus
GDKGSQGESGIQGR

SEQ ID NO: 86 - protein, 15 aa, Gallus gallus
GDKGSQGESGIQGRK

SEQ ID NO: 87 - protein, 18 aa, Gallus gallus
GDKGSQGESGIQGRKGEK

SEQ ID NO: 88 - protein, 20 aa, Gallus gallus
GDKGSQGESGIQGRKGEKGR

SEQ ID NO: 89 - protein, 12 aa, Gallus gallus
KGSQGESGIQGR

SEQ ID NO: 90 - protein, 11 aa, Gallus gallus
GSQGESGIQGR

SEQ ID NO: 91 - protein, 12 aa, Gallus gallus
GSQGESGIQGRK

SEQ ID NO: 92 - protein, 15 aa, Gallus gallus
GSQGESGIQGRKGEK

SEQ ID NO: 93 - protein, 17 aa, Gallus gallus
GSQGESGIQGRKGEKGR

SEQ ID NO: 94 - protein, 19 aa, Gallus gallus
KGEKGRQGNPGLQGTEGLR

SEQ ID NO: 95 - protein, 25 aa, Gallus gallus
KGEKGRQGNPGLQGTEGLRGEQGEK

SEQ ID NO: 96 - protein, 18 aa, Gallus gallus
GEKGRQGNPGLQGTEGLR

SEQ ID NO: 97 - protein, 27 aa, Gallus gallus
GEKGRQGNPGLQGTEGLRGEQGEKGEK

SEQ ID NO: 98 - protein, 14 aa, Gallus gallus
GRQGNPGLQGTEGL

SEQ ID NO: 99 - protein, 15 aa, Gallus gallus
GRQGNPGLQGTEGLR

SEQ ID NO: 100 - protein, 21 aa, Gallus gallus
GRQGNPGLQGTEGLRGEQGEK

SEQ ID NO: 101 - protein, 24 aa, Gallus gallus
GRQGNPGLQGTEGLRGEQGEKGEK

SEQ ID NO: 102 - protein, 26 aa, Gallus gallus
GRQGNPGLQGTEGLRGEQGEKGEKGD

SEQ ID NO: 103 - protein, 13 aa, Gallus gallus
QGNPGLQGTEGLR

SEQ ID NO: 104 - protein, 19 aa, Gallus gallus
QGNPGLQGTEGLRGEQGEK

SEQ ID NO: 105 - protein, 22 aa, Gallus gallus
QGNPGLQGTEGLRGEQGEKGEK

SEQ ID NO: 106 - protein, 24 aa, Gallus gallus
QGNPGLQGTEGLRGEQGEKGEKGD

SEQ ID NO: 107 - protein, 16 aa, Gallus gallus
RGEQGEKGEKGDPGIR

SEQ ID NO: 108 - protein, 6 aa, Gallus gallus
GEQGEK

SEQ ID NO: 109 - protein, 15 aa, Gallus gallus
GEQGEKGEKGDPGIR

SEQ ID NO: 110 - protein, 9 aa, Gallus gallus
GEKGDPGIR

SEQ ID NO: 111 - protein, 6 aa, Gallus gallus
GDPGIR

(19) United States
(12) Patent Application Publication
(10) Pub. No.: US 2024/0415757 A1
(43) Pub. Date: Dec. 19, 2024

(54) ANIMAL-FREE COSMETIC COLLAGENS

(71) Applicant: Geltor, Inc., Emeryville, CA (US)

(72) Inventors: Nikolay OUZOUNOV, Lafayette, CA (US); Jeffrey R. MELLIN, Richmond, CA (US); Julia CO, Oakland, CA (US); José M. MELO BARCELOS, San Jose, CA (US)

(21) Appl. No.: 18/420,585

(22) Filed: Jan. 23, 2024

Related U.S. Application Data
(63) Continuation of application No. PCT/US2022/038590, filed on Jul. 27, 2022.
(60) Provisional application No. 63/226,425, filed on Jul. 28, 2021.

(51) Int. Cl. A61K 8/65; A61Q 19/00; A61Q 19/08; C07K 14/78

(52) U.S. Cl. CPC A61K 8/65; A61Q 19/00; A61Q 19/007; A61Q 19/08; C07K 14/78

(57) ABSTRACT

Provided herein are cosmetic (e.g., topical) formulations comprising non-naturally occurring polypeptides, such as non-naturally occurring polypeptides comprising one or more truncations relative to full-length collagens. The cosmetic (e.g., topical) formulations provided herein may be used in personal care products (e.g., cosmetics).

### CROSS-REFERENCE

This application is a continuation of International Application No. PCT/US2022/038590, filed Jul. 27, 2022, which claims the benefit of U.S. Provisional Application No. 63/226,425, filed Jul. 28, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 19, 2024, is named 57607_718_301_SL.xml and is 107,138 bytes in size.

### BACKGROUND

Collagen is one of the most abundant proteins found in various connective tissues in the body including tendons, ligaments, skin, and hair. Collagens or collagen supplements are popular in medical, cosmetic, and/or health purposes (e.g., stimulating skin growth, promoting wound healing, strengthening nails or joints, etc.). Collagens for most collagen supplements are derived from animals as a byproduct of the animal processing industry. Yet, such animal-derived collagens may increase the risk of illness transmission as well as allergies. Moreover, certain consumers are generally interested in animal-free products for a variety of other reasons. Thus, there remains a need for improved compositions and methods of collagens derived from non-animal sources.

### SUMMARY

In one aspect of the disclosure, a cosmetic formulation is provided comprising: a polypeptide comprising or consisting of an amino acid sequence having at least 80% sequence identity to SEQ ID NO: 32, or a polypeptide comprising or consisting of an amino acid sequence having at least 80% sequence identity to a truncate of SEQ ID NO: 32, wherein the cosmetic formulation is selected from the group consisting of: a cream, a gel, a gel cream, an oil, an ointment, a serum, a foam, a lotion, a paste, a balm, a solution, a suspension, and a powder. In some cases, the cosmetic formulation is a cream, a gel cream, or a powder.

In another aspect, a cosmetic formulation is provided comprising: (i) a polypeptide comprising or consisting of an amino acid sequence having at least 80% sequence identity to SEQ ID NO: 32, or a polypeptide comprising or consisting of an amino acid sequence having at least 80% sequence identity to a truncate of SEQ ID NO: 32; and (ii) one or more additional ingredients selected from the group consisting of: levulinic acid, polyglyceryl-3 methylglucose distearate, glyceryl undecylenate, Simmondsia chinensis (Jojoba) seed oil, polyacrylate cross-polymer, squalane, sodium hyaluronate, acrylic acid polymers (carbomers), pentylene glycol, sodium lauryl sulfoacetate, sodium oleoyl sarcosinate, sodium oleate, Ricinus communis (castor) seed oil, Copernicia cerifera (Carnauba) wax, Candelilla wax, Theobroma cacao (Cocoa) Seed Butter, isononyl isononanoate, ozokerite, isopropyl titanium triisostearate, polyhydroxystearic acid, iron oxide, titanium dioxide, sodium levulinate, and hydroxypropyl guar.

In any of the preceding embodiments, the polypeptide comprises or consists of an amino acid sequence having at least 85% sequence identity to SEQ ID NO: 32, or the polypeptide comprises or consists of an amino acid sequence having at least 85% sequence identity to a truncate of SEQ ID NO: 32. In any of the preceding embodiments, the polypeptide comprises or consists of an amino acid sequence having at least 90% sequence identity to SEQ ID NO: 32, or the polypeptide comprises or consists of an amino acid sequence having at least 90% sequence identity to a truncate of SEQ ID NO: 32. In any of the preceding embodiments, the polypeptide comprises or consists of an amino acid sequence having at least 95% sequence identity to SEQ ID NO: 32, or the polypeptide comprises or consists of an amino acid sequence having at least 95% sequence identity to a truncate of SEQ ID NO: 32. In any of the preceding embodiments, the polypeptide comprises or consists of an amino acid sequence having at least 98% sequence identity to SEQ ID NO: 32, or the polypeptide comprises or consists of an amino acid sequence having at least 98% sequence identity to a truncate of SEQ ID NO: 32. In any of the preceding embodiments, the polypeptide comprises or consists of an amino acid sequence having 100% sequence identity to SEQ ID NO: 32, or the polypeptide comprises or consists of an amino acid sequence having 100% sequence identity to a truncate of SEQ ID NO: 32. In any of the preceding embodiments, the truncate of SEQ ID NO: 32 comprises an N-terminal truncation, a C-terminal truncation, or both, relative to SEQ ID NO: 32. In any of the preceding embodiments, the N-terminal truncation is an N-terminal truncation of 50 amino acids to 750 amino acids relative to SEQ ID NO: 32. In any of the preceding embodiments, the C-terminal truncation is a C-terminal truncation of 50 amino acids to 600 amino acids relative to SEQ ID NO: 32. In any of the preceding embodiments, the polypeptide comprises or consists of an amino acid sequence having at least 80% sequence identity to SEQ ID NO: 8. In any of the preceding embodiments, the polypeptide comprises or consists of an amino acid sequence having at least 90% sequence identity to SEQ ID NO: 8. In any of the preceding embodiments, the polypeptide comprises or consists of an amino acid sequence having at least 95% sequence identity to SEQ ID NO: 8. In any of the preceding embodiments, the polypeptide comprises or consists of an amino acid sequence having at least 98% sequence identity to SEQ ID NO: 8. In any of the preceding embodiments, the polypeptide comprises or consists of an amino acid sequence having 100% sequence identity to SEQ ID NO: 8. In any of the preceding embodiments, the polypeptide has a total truncation of 50 amino acids to 1250 amino acids. In any of the preceding embodiments, the polypeptide is at least 50 amino acids in length. In any of the preceding embodiments, the polypeptide is 50 amino acids to 250 amino acids in length. In any of the preceding embodiments, the polypeptide does not comprise one or more of: a laminin G domain, a Von Willebrand factor type A (vWA) domain, and a fibrillar collagen C-terminal domain. In any of the preceding embodiments, the polypeptide comprises one or more collagen triple helix repeats. In any of the preceding embodiments, the polypeptide is monomeric. In any of the preceding embodiments, the polypeptide does not form a stable triple helix structure of a naturally occurring collagen. In any of the preceding embodiments, the polypeptide is substantially free of other collagen chains. In any of the preceding embodiments, the polypeptide has a non-naturally occurring level of hydroxylation relative to a naturally-occurring collagen. In any of the preceding embodiments, fewer than 10% of prolines present in the polypeptide are hydroxylated. In any of the preceding embodiments, the polypeptide is non-hydroxylated. In any of the preceding embodiments, the polypeptide has a non-naturally occurring level of glycosylation relative to a naturally-occurring collagen. In any of the preceding embodiments, the polypeptide comprises less than 5 wt. % glycosylation. In any of the preceding embodiments, the polypeptide is present in the cosmetic formulation at an amount of 0.001% to 30% w/w. In any of the preceding embodiments, the cosmetic formulation is formulated for topical application. In any of the preceding embodiments, the cosmetic formulation is formulated for application to the skin or hair of an individual. In any of the preceding embodiments, the cosmetic formulation further comprises a topical carrier. In any of the preceding embodiments, the topical carrier is selected from the group consisting of: a liposome, a biodegradable microcapsule, a lotion, a spray, an aerosol, a dusting powder, a biodegradable polymer, mineral oil, triglyceride oil, silicone oil, glycerin, glycerin monostearate, an alcohol, an emulsifying agent, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, and water. In any of the preceding embodiments, the cosmetic formulation further comprises a preservative. In any of the preceding embodiments, the preservative is selected from the group consisting of: tocopherol, diiodomethyl-p-tolylsulfone, 2-bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-ethylbicyclooxazolidine, phenoxyethanol, butylene glycol, 1,2 hexanediol, methyl paraben, sorbic acid, Germaben® II, rosemary extract, and ethylenediaminetetraacetic acid (EDTA).

In another aspect, a personal care product is provided, comprising the cosmetic formulation of any of the preceding embodiments. In some embodiments, the personal care product is selected from the group consisting of: a mask, a skin cleaners, a cleansing cream, a cleansing lotion, a facial lotion, a body lotion, a shower gel, an antiperspirant, a deodorant, a shave cream, a depilatory, a face oil, a lip oil, a body oil, a facial cleanser, a cleansing milk, a cleansing pad, a facial wash, a facial cream, a body cream, a facial moisturizer, a body moisturizer, a facial serum, a facial mask, a body mask, a facial toner, a facial mist, an eye cream, an eye serum, an exfoliator formula, a lip balm, a lipstick, a hair shampoo, a hair conditioner, a body shampoo, a hair serum, a scalp serum, a hair mist, a hair spray, a foundation, a tinted multifunctional cream, an eye shadow, a concealer, a mascara, and any combination thereof.

In yet another aspect, a method of promoting, improving, and/or maintaining youthful skin of an individual is provided, the method comprising: applying the cosmetic formulation or the personal care product of any of the preceding embodiments to the skin of the individual, thereby promoting, improving, and/or maintaining youthful skin of the individual. In some embodiments, promoting, improving, and/or maintaining youthful skin of the individual comprises improving firmness of the skin of the individual. In some embodiments, improving firmness of the skin of the individual comprises increasing skin firmness (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on measuring the resistance of the skin to negative pressure (e.g., using a Cutometer®). In some embodiments, promoting, improving, and/or maintaining youthful skin of the individual comprises improving elasticity of the skin of the individual. In some embodiments, improving elasticity of the skin of the individual comprises increasing skin elasticity (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on measuring the ability of the skin to return to its original position after deformation (e.g., using a Cutometer®). In some embodiments, promoting, improving, and/or maintaining youthful skin of the individual comprises improving brightness of the skin of the individual. In some embodiments, improving brightness of the skin of the individual comprises increasing brightness of the skin (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% as determined by an expert clinical grader. In some embodiments, promoting, improving, and/or maintaining youthful skin of the individual comprises improving hydration of the skin of the individual. In some embodiments, improving hydration of the skin of the individual comprises increasing skin hydration (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on capacitance measurement of the skin (e.g., using a Corneometer®). In some embodiments, promoting, improving, and/or maintaining youthful skin of the individual comprises improving tactile texture of the skin of the individual. In some embodiments, promoting, improving, and/or maintaining youthful skin of the individual comprises improving collagen content of the skin of the individual. In some embodiments, promoting, improving, and/or maintaining youthful skin of the individual comprises improving elastin content of the skin of the individual. In some embodiments, promoting, improving, and/or maintaining youthful skin of the individual comprises improving redness of the skin of the individual. In some embodiments, improving redness of the skin of the individual comprises decreasing redness of the skin (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% as determined by an expert clinical grader. In some embodiments, promoting, improving, and/or maintaining youthful skin of the individual comprises improving visual texture of the skin of the individual. In some embodiments, promoting, improving, and/or maintaining youthful skin of the individual comprises improving fine lines and/or wrinkles of the skin of the individual. In some embodiments, improving fine lines and/or wrinkles of the skin of the individual comprises decreasing fine lines and/or wrinkles (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% as determined by an expert clinical grader. In some embodiments, promoting, improving, and/or maintaining youthful skin of the individual comprises improving epidermal thickness of the skin of the individual. In some embodiments, improving epidermal thickness of the skin of the individual comprises increasing epidermal thickness (e.g., relative to the skin prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% as measured by reflectance confocal microscopy (e.g., using a Vivascope®). In some embodiments, after the applying, keratinocyte growth and/or regeneration in the skin is increased (e.g., relative to prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In some embodiments, after the applying, collagen production in the skin is increased (e.g., relative to prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In some embodiments, after the applying, fibroblast migration, proliferation, and/or adhesion in the skin is increased (e.g., relative to prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In some embodiments, after the applying, keratinocyte viability after exposure to urban dust is increased (e.g., relative to prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In some embodiments, after the applying, expression of one or more genes involved in a signaling pathway selected from the group consisting of: VEGFA/VEGFR2 signaling pathway, focal adhesion signaling pathway, endothelin signaling pathway, EGF/EGFR signaling pathway, TGF-beta signaling pathway, and any combination thereof, is increased. In some embodiments, the one or more genes involved in VEGFA/VEGFR2 signaling pathway is selected from the group consisting of: MYOC1, FLII, ROCK1, ROCK2, CLTC, LIMK 1, EGR1, and any combination thereof. In some embodiments, the one or more genes involved in focal adhesion signaling pathway is selected from the group consisting of: ITGA3, TNC, LAMC1, FLNA, TLN1, ZYX, DIAPH1, and any combination thereof. In some embodiments, the one or more genes involved in endothelin signaling pathway is selected from the group consisting of: TRIOBP, WNK1, MMP2, VCAN, ACTA2, GNA12, EGR1, and any combination thereof. In some embodiments, the one or more genes involved in EGF/EGFR signaling pathway is selected from the group consisting of: ATXN2, JAK1, RPS6KA2, ROCK1, SHC1, IQGAP1, PLCG1, and any combination thereof. In some embodiments, the one or more genes involved in TGF-beta signaling pathway is selected from the group consisting of: SMURF1, SPTBN1, PAK2, ROCK1, SHC1, TGFBR3, TGFBR1, and any combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1 depicts alignment of non-naturally occurring polypeptides of the disclosure with corresponding naturally occurring collagens. FIG. 1 discloses SEQ ID NO: 33 (a subsection of SEQ ID NO: 31).

FIG. 2 depicts alignment of non-naturally occurring polypeptides of the disclosure with corresponding naturally occurring collagens. FIG. 2 discloses SEQ ID NO: 34 (a subsection of SEQ ID NO: 32).

FIG. 3 depicts an image of two SDS-PAGE gels showing bands of collagen proteins in supernatant samples from microbial cell cultures. The identities of each protein are indicated above each band.

FIGS. 4A-4C depict images of SDS-PAGE gels showing bands of non-naturally occurring polypeptides of the disclosure before and after pH 3.0 treatment.

FIG. 5 depicts the effect of pH, polypeptide concentration, and temperature on the dissolution time of an exemplary non-naturally occurring polypeptide of the disclosure.

FIG. 6 depicts the effect of pH, polypeptide concentration, and temperature on turbidity of a solution containing an exemplary non-naturally occurring polypeptide of the disclosure.

FIG. 7A and FIG. 7B depict the effect of pH and temperature on the stability of a powder containing an exemplary non-naturally occurring polypeptide of the disclosure as measured by total amount of polypeptide.

FIG. 8A and FIG. 8B depict the effect of pH and temperature on the stability of a powder containing an exemplary non-naturally occurring polypeptide of the disclosure as measured by % full-length polypeptide/degradation.

FIGS. 9A-9C depict the effect of pH and temperature on the viscosity of a solution containing an exemplary non-naturally occurring polypeptide of the disclosure.

FIGS. 10A-10C depict the effect of pH and temperature on the viscosity of a solution containing an exemplary non-naturally occurring polypeptide of the disclosure.

FIG. 11 depicts increased firmness of a skin model substrate treated with an exemplary non-naturally occurring polypeptide of the disclosure versus a benchmark.

FIGS. 12A-12C depict viability of an immortalized human keratinocyte cell line, human primary fibroblasts, and human primary keratinocytes after exposure to an exemplary non-naturally occurring polypeptide of the disclosure.

FIG. 13 depicts a dose-dependent increase in proliferation of human primary keratinocytes after exposure to an exemplary non-naturally occurring polypeptide of the disclosure.

FIG. 14 depicts a dose-dependent increase in collagen I production by primary human fibroblasts after exposure to an exemplary non-naturally occurring polypeptide of the disclosure.

FIG. 15 depicts wound healing activity of human dermal fibroblasts after exposure to an exemplary non-naturally occurring polypeptide of the disclosure.

FIG. 16 depicts viability of an immortalized human keratinocyte cell line pre-treated with an exemplary non-naturally occurring polypeptide of the disclosure after exposure to urban dust.

FIG. 17 depicts the antioxidant capacity of an exemplary non-naturally occurring polypeptide of the disclosure.

### DETAILED DESCRIPTION

### Definitions

The terminology used herein is for the purpose of describing particular cases only and is not intended to be limiting. As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The terms “about” or “approximately” mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual”, “patient”, or “subject” are used interchangeably herein. None of the terms require or are limited to a situation characterized by the supervision (e.g., constant or intermittent) of a health care worker (e.g., a doctor, a registered nurse, a nurse practitioner, a physician's assistant, an orderly, or a hospice worker).

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of”. The phrase “consisting essentially of” is used herein to require the specified feature(s) as well as those which do not materially affect the character or function of the claimed disclosure. As used herein, the term “consisting” is used to indicate the presence of the recited feature alone.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as any individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The term “truncated collagen” as used herein generally refers to a polypeptide that is smaller than a full-length (e.g., natural) collagen wherein one or more portions of the full-length (e.g., natural) collagen is not present. The non-naturally occurring polypeptides provided herein may be truncated at the C-terminal end, the N-terminal end, truncated by removal of internal portion(s) of the full-length collagen sequence (e.g., an internal truncation), truncated at both the C-terminal end and the N-terminal end, or may have one or both of a C-terminal truncation and an N-terminal truncation as well as an internal truncation. In a non-limiting embodiment, a truncated collagen may comprise an amino acid sequence according to SEQ ID NO: 2, or a homolog thereof. In another non-limiting embodiment, a truncated collagen may comprise an amino acid sequence according to SEQ ID NO: 8, or a homolog thereof.

When used in reference to an amino acid position, a “truncation” is inclusive of said amino acid position. For example, an N-terminal truncation at amino acid position 100 relative to a full-length polypeptide means a truncation of 100 amino acids from the N-terminus of the full-length polypeptide (i.e., the truncated polypeptide is missing amino acid positions 1 through 100 of the full-length polypeptide). Similarly, a C-terminal truncation at amino acid position 901 of a full-length polypeptide (assuming a 1000 amino acid full-length polypeptide) means a truncation of 100 amino acids from the C-terminus (i.e., the truncated polypeptide is missing amino acid positions 901 through 1000 of the full-length polypeptide). Similarly, an internal truncation at amino acid positions 101 and 200 means an internal truncation of 100 amino acids of the full-length polypeptide (i.e., the truncated polypeptide is missing amino acid positions 101 to 200 of the full-length polypeptide).

The section headings used herein are for organizational purposes only and are not to be construed as limiting the subject matter described.

Provided in certain embodiments herein are, by way of non-limiting example, compositions, methods, and systems for manufacturing non-naturally occurring polypeptides, such as, e.g., animal-free collagen polypeptides or collagen-like polypeptides, as well as collagen fragments, and/or truncated collagens, such as that are expressed in and/or by genetically engineered microorganisms. Thus, in various aspects of the disclosure, the non-naturally occurring polypeptides provided herein include collagen or collagen-like polypeptides, recombinant collagens, collagen fragments, or truncated collagens. In certain embodiments, the non-naturally occurring polypeptides described herein (e.g., recombinant collagens, collagen fragments, or truncated collagens) are derived from any suitable source, such as from mammalian or non-mammalian sources. For example, in some embodiments, the non-naturally occurring polypeptides described herein (e.g., recombinant collagens, collagen fragments, or truncated collagens), or at least a portion thereof, are derived from (e.g., modified, truncated, fragments of, or the like) collagens of a bird or an avian animal (e.g., Gallus gallus collagen), a freshwater- or saltwater-fish (e.g., Acipenser schrenckii collagen), or any combination thereof.

The non-naturally occurring polypeptides provided herein are not normally found in nature. Generally, the non-naturally occurring polypeptides described herein exhibit one or more differences from naturally occurring collagens. In certain aspects, the non-naturally occurring polypeptides provided herein may have a different amino acid sequence from naturally occurring polypeptides (e.g., a truncated collagen). In some cases, the non-naturally occurring polypeptides may have a different structure from a naturally occurring collagen. The quaternary structure of natural collagen is a triple helix, typically composed of three polypeptides. In some aspects, the non-naturally occurring polypeptides described herein may not have or may not form a quaternary structure of natural collagen. For example, in some instances, the non-naturally occurring polypeptides described herein may not form the stable triple helical structure of naturally occurring collagen. In certain instances, of the three polypeptides that form natural collagen, two are usually identical and are designated as the alpha chain. The third polypeptide is designated as the beta chain. In certain instances, a typical natural collagen can be designated as AAB, wherein the collagen is composed of two alpha (“A”) strands and one beta (“B”) strand. In some aspects, the non-naturally occurring polypeptides described herein do not have the AAB structure of natural collagen. In some instances, the non-naturally occurring polypeptides described herein are free from or substantially free from different collagen chains (e.g., a non-naturally occurring polypeptide described herein may comprise an alpha chain collagen and may be free or substantially free from a beta chain collagen). In some aspects, the non-naturally occurring polypeptides described herein are monomeric and/or do not form multimeric structures. In other aspects, the non-naturally occurring polypeptides described herein may, in some instances, form multimeric structures with identical monomers (e.g., homodimers, homotrimers, etc.).

In some aspects, the non-naturally occurring polypeptides are recombinant polypeptides (e.g., prepared recombinantly in a host cell). The non-naturally occurring polypeptide is, in one embodiment, a truncated collagen. Other non-naturally occurring collagen polypeptides include chimeric collagens. A chimeric collagen is a polypeptide wherein one portion of a collagen polypeptide is contiguous with a portion of a second collagen polypeptide. For example, a collagen molecule comprising a portion of a collagen from one species contiguous with a portion of a collagen from another species is a chimeric collagen. In another embodiment, the non-naturally occurring polypeptide comprises a fusion polypeptide that includes additional amino acids such as a secretion tag, histidine tag, green fluorescent protein, protease cleavage site, GEK repeats, GDK repeats, and/or beta-lactamase.

In some embodiments, the non-naturally occurring polypeptides (e.g., recombinant polypeptides) provided herein have a non-naturally occurring level of glycosylation, for example, relative to a corresponding natural collagen or naturally present collagen. For example, in some embodiments, the non-naturally occurring polypeptide (e.g., recombinant polypeptide) comprises less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, less than about 0.9 wt. %, less than about 0.8 wt. %, less than about 0.7 wt. %, less than about 0.6 wt. %, less than about 0.5 wt. %, less than about 0.4 wt. %, less than about 0.3 wt. %, less than about 0.2 wt. %, or less than about 0.1 wt. % glycosylation. Alternatively and/or additionally, the non-naturally occurring polypeptide (e.g., recombinant polypeptide) comprises less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% of total glycosylation of the corresponding natural collagen or naturally present collagen. For example, where the naturally present collagen ABC from a species XYZ has 20 glycosylations (throughout the full length of the collagen ABC or a portion thereof), it is contemplated that the non-naturally occurring polypeptide (e.g., recombinant polypeptide) comprises less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1 glycosylations. In some embodiments, those lower levels of glycosylation can be specific to one or more types of glycosylation (e.g., O-glycosylation or N-glycosylation, etc.) and/or the glycosylation residues (e.g., galactosylhydroxylysine (Gal-Hyl), glucosyl galactosylhydroxylysine (GlcGal-Hyl), etc.). Non-naturally occurring polypeptides produced recombinantly (e.g., in a recombinant host cell), in some instances, may have a glycosylation level and/or a glycosylation pattern that differs from naturally occurring collagen.

In some aspects, a non-naturally occurring polypeptide provided herein has a non-naturally occurring amount of hydroxyprolines. In some cases, a non-naturally occurring polypeptide provided herein lacks hydroxyprolines. In some cases, a non-naturally occurring polypeptide provided herein comprises fewer hydroxyprolines than a naturally-occurring collagen. Hydroxyprolines include, without limitation, 3-hydroxyproline, 4-hydroxyproline, and 5-hydroxyproline. In some cases, less than about 50% (e.g., less than about 45%, less than about 40%, less than about 35%, less than about 30%, less than about 25%, less than about 20%, less than about 15%, less than about 10%, or less) of the prolines present in the amino acid sequence of a non-naturally occurring polypeptide provided herein are hydroxyprolines. In some aspects, a non-naturally occurring polypeptide produced recombinantly (e.g., in a recombinant host cell) may have fewer hydroxyprolines than a naturally occurring collagen. In some cases, a recombinant polypeptide as provided herein is recombinantly expressed in a recombinant host cell (e.g., bacterial cell, yeast cell, fungal cell) that lacks an enzyme that hydroxylates one or more amino acids (e.g., proline) of the recombinant polypeptide. In some cases, a recombinant polypeptide as provided herein is recombinantly expressed in a host cell (e.g., bacterial cell, yeast cell, fungal cell) that lacks prolyl 4-hydroxylase and/or prolyl 3-hydroxylase.

In some aspects, the non-naturally occurring polypeptides provided herein lack or substantially lack lysyl oxidation. Lysyl oxidation involves the conversion of lysine residues into highly reactive aldehydes that can form cross-links with other proteins. Naturally occurring collagens may have some level of lysyl oxidation. Thus, the non-naturally occurring polypeptides may be different from natural collagens in that they lack or substantially lack lysyl oxidation. In some cases, less than about 50% (e.g., less than about 45%, less than about 40%, less than about 35%, less than about 30%, less than about 25%, less than about 20%, less than about 15%, less than about 10%, or less) of the lysines present in the amino acid sequence of a non-naturally occurring polypeptide provided herein are oxidized.

Generally, the non-naturally occurring polypeptides provided herein (e.g., truncated collagens) may have a function and/or provide a benefit (e.g., as provided herein) similar or substantially similar to that of a natural or a full-length collagen. In some cases, the non-naturally occurring polypeptides provided herein (e.g., truncated collagens) may have improved or increased function and/or benefit (e.g., as provided herein) as compared to a natural or a full-length collagen. In some embodiments, the non-naturally occurring polypeptides provided herein may have one or more different functions as compared to a natural or a full-length collagen.

The non-naturally occurring polypeptides disclosed herein often have advantageous properties related to their monomeric structure and/or lack of amino acids capable of cross-linking with other collagen strands, e.g., the lack of hydroxyproline residues. In addition, collagen hydrolysates of the non-naturally occurring polypeptides disclosed herein are also produced with increased solubility as compared to full-length or natural collagens. Moreover, monomeric structures, as opposed to natural triple helix collagens, are more readily digestible and bioavailable, or broken down by digestive proteases. Other advantageous properties include improved physical properties in liquid compositions and in purification processes, since full-length or natural collagens or collagen strands interact to form stronger structures that can precipitate due to the presence of hydroxyproline residues.

In certain preferred embodiments, the non-naturally occurring polypeptides provided herein (e.g., truncated collagens) comprise an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to at least a portion of the naturally existing mammalian or non-mammalian collagens from which those are derived from. In some instances, a portion or portions of a natural amino acid sequence is deleted, but the remainder of the sequence is substantially similar or identical to the natural amino acid sequence. In certain exemplary embodiments, the non-naturally occurring polypeptide has an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a Gallus gallus Type 21 alpha 1 collagen or a truncate or a fragment thereof. In another example, the non-naturally occurring polypeptide has an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a Acipenser schrenckii Type 2 alpha 1 collagen or a truncate or a fragment thereof.

In some embodiments, the recombinant polypeptide is a truncated collagen. In certain instances, a truncated collagen is a polypeptide that is smaller than a full-length (e.g., natural) collagen wherein one or more portions (e.g., internal and/or terminal portion(s)) of the full-length (e.g., natural) collagen is not present. In various instances, the non-naturally occurring polypeptides provided herein (e.g., truncated collagens) are truncated at the C-terminal end, the N-terminal end, truncated by removal of internal portion(s) of the full-length collagen polypeptide (e.g., internal truncation), truncated at both the C-terminal end and the N-terminal end, or comprise one or both of a C-terminal truncation and an N-terminal truncation as well as an internal truncation. In some instances, the non-naturally occurring polypeptide is a fragment of a naturally occurring collagen that retains at least about 50%, at least about 60%, at least about 70%, at least about 80%, or at least about 90% of a function (e.g., of interest) of natural or naturally-present corresponding collagens. In some instances, the term truncated collagen is interchangeably used with the term collagen fragment. In some instances, the truncated collagen includes any contiguous collagen fragments that are at least about 10%, at least about 20%, at least about 30%, at least about 40%, at least about 50%, at least about 60%, at least about 70%, or at least about 80% of full-length natural or naturally-present corresponding collagens. In some embodiments, the truncation is an internal truncation, a truncation at the N-terminal portion of the collagen, a truncation at the C-terminal portion of the collagen, a truncation of an internal portion, or a truncation at both the C-terminal end and the N-terminal end. A truncated collagen provided herein may be truncated by 50 amino acids to 1250 amino acids, 50 amino acids to 1200 amino acids, 50 amino acids to 1150 amino acids, 50 amino acids to 1100 amino acids, 50 amino acids to 1050 amino acids, 50 amino acids to 1000 amino acids, 50 amino acids to 950 amino acids, 50 amino acids to 900 amino acids, 50 amino acids to 850 amino acids, 50 amino acids to 800 amino acids, 50 amino acids to 750 amino acids, 50 amino acids to 700 amino acids, 50 amino acids to 650 amino acids, 50 amino acids to 600 amino acids, 50 amino acids to 550 amino acids, 50 amino acids to 500 amino acids, 50 amino acids to 450 amino acids, 50 amino acids to 400 amino acids, 50 amino acids to 350 amino acids, 50 amino acids to 300 amino acids, 50 amino acids to 250 amino acids, 50 amino acids to 200 amino acids, 50 amino acids to 150 amino acids, or 50 amino acids to 100 amino acids (e.g., relative to a full-length collagen). In another embodiment, a truncated collagen is truncated by 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900, 950, 1000, 1050, 1100, 1150, 1200, or 1250 amino acids (e.g., relative to a full-length collagen).

In some embodiments, a polypeptide provided herein (e.g., amino acid sequence thereof) may be truncated at the C-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein (e.g., amino acid sequence thereof) may be truncated at the C-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be truncated at the N-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be truncated at the N-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be truncated at both the N-terminal end and the C-terminal end relative to a full-length collagen. In some instances, a polypeptide provided herein may be truncated at the N-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like; and may be truncated at the C-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be truncated at the N-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids; and may be truncated at the C-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be internally truncated (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be internally truncated (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

A non-naturally occurring polypeptide (e.g., truncated collagen) disclosed herein may comprise a truncation relative to a full-length (e.g., natural) collagen. In some embodiments, a truncated collagen disclosed herein may comprise a truncation relative to a full-length (e.g., natural) chicken (Gallus gallus) type 21 alpha 1 collagen (e.g., SEQ ID NO: 31). In some embodiments, a truncated collagen disclosed herein may comprise the amino acid sequence of SEQ ID NO: 31, or an amino acid sequence having at least about 70% sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 31, with an N-terminal truncation, a C-terminal truncation, an internal truncation, or a combination thereof. In some embodiments, a truncated collagen disclosed herein may comprise a truncation relative to a full-length (e.g., natural) Japanese sturgeon (Acipenser schrenckii) type 2 alpha 1 collagen (e.g., SEQ ID NO: 32). In some embodiments, a truncated collagen disclosed herein may comprise the amino acid sequence of SEQ ID NO: 32, or an amino acid sequence having at least about 70% sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 32, with an N-terminal truncation, a C-terminal truncation, an internal truncation, or a combination thereof. Non-limiting examples of full-length (e.g., natural) collagens are provided in Table 1 below.

In some cases, a polypeptide provided herein may be at least 50 amino acids, at least 75 amino acids, at least 100 amino acids, at least 125 amino acids, at least 150 amino acids, at least 175 amino acids, at least 200 amino acids, at least 225 amino acids, at least 250 amino acids, at least 275 amino acids, at least 300 amino acids, at least 350 amino acids, at least 400 amino acids, at least 450 amino acids, or at least 500 amino acids in length.

In other embodiments, polypeptides as disclosed herein may be truncated collagen polypeptides comparable to fish collagens, including from other species of sturgeon, or from other species producing roe suitable for caviar, including salmon, steelhead, trout, lumpfish, whitefish, or carp, as well as other fish such as tilapia and sharks. Suitable comparable sequences from Acipenser schrenckii (Japanese sturgeon) include NCBI accession numbers BAO58965.1, BAO58966.1, BAO58967.1, BAT51012.1, BAR72360.1, BAR72359.1, BAR72358.1, BAR72357.1 and BAR72356.1. Suitable sequences from Acipenser ruthenus (Sterlet sturgeon) include NCBI accession numbers A0A444UGW0, A0A444TZM6, A0A444UC45, A0A444UC53, A0A662YTX1, A0A662Z270, A0A662YZ39, A0A444U1F5, A0A444UJK3, A0A444UNU0, X5HZZ7, X5IHC1, A0A444UPK8, A0A444UBS1, A0A444UYQ7, A0A444TWQ3, A0A444ULY4, A0A444TZ23, A0A662YS48, A0A444U4C8, A0A444UD64, A0A662YX10, A0A662YXI2, A0A444TXQ4, A0A444TZ42, A0A444U8N8, A0A444UJU3, A0A444UQ51, A0A444U2T2, A0A662YJ50, A0A444V1V9, A0A444V113, A0A662YWR6, A0A662YW91, A0A444U5J5, A0A662YR93, A0A444UJB0, A0A444UFS4, A0A444UVK2, A0A444UJU1, A0A444ULY9, A0A444UKA7, A0A444U5L7, A0A444V6M4, A0A444V788, A0A444UFS9, A0A444UVP7, A0A444U4D9, A0A444UHN6, A0A662YJC1, A0A444V1E8, A0A444UPM0, A0A662YU87, A0A444TZS8, A0A444U200, A0A444V2E3, A0A662YXD3, A0A662YQA4, A0A444U1H9, A0A444V715, A0A444UFX8, A0A444V7B8, A0A444U2K4, A0A444V762, A0A444UQ49, A0A662YMD3, A0A662YWF2, A0A444UE44, A0A444UAR6, A0A444UX46, A0A444U5P4, A0A662YRG8, A0A444USC3, A0A444UK09, A0A444UNQ7, A0A444UN69, A0A444V5D9, E6Y298, A0A444TZY1, A0A444TYS0, and E6Y299.

In other embodiments, polypeptides may be truncated collagen polypeptides comparable to chicken collagens, or other poultry collagens, such as from domestic fowls, including chickens, turkeys, geese, and ducks. Suitable comparable sequences from Gallus gallus (chicken) include NCBI accession numbers V9GZR2, Q9PSS5, A0A3Q2UDI3, Q90802, A0A1D5PNH7, Q4TZW6, Q90803, Q91014, A0A1D5PPI0, A0A1D5P1A5, A0A3Q2U6K2, A0A3Q2U8F9, Q90689, A0A3Q2U3U6, P13731, A0A1D5PFE0, A0A3Q2TXZ7, Q5FY72, A0A1D5PR16, A0A1D5PKR6, F1NDF5, Q90589, P08125, F1NRH2, P32017, A0A1D5PW49, Q90800, P12108, E1C353, Q7LZR2, P02460, A0A1L1RNI7, Q90796, P12106, F1NQ20, Q919K3, P20785, A0A1D5PWN6, P15988, P12105, F1NIL4, 093419, P02467, A0A5H1ZRJ7, A0A1D5PKQ4, A0A5H1ZRK9, Q90W37, A0A1D5NY11, A0A1D5P959, P02457, A0A1DSPYU1, A0A1D5PE57, Q90ZA0, Q90584, A0A1L1RZW7, A0A1D5NVM0, A0A1D5P8P3, F1NIP0, F1P2Q3, A0A1D5PE74, Q9IAU4, A0A3Q2TTC1, F1NHH4, P32018, A0A1D5P0F4, R4GHP9, A0A3Q2UD12, A0A3Q2UMJ2, A0A3Q2U4U7, F1NX22, A0A1D5P818, A0A1L1RPW4, P13944, P15989, F1P2F0, A0A1D5PGD5, and A0A3Q3AR07.

TABLE 1
Full-length collagen amino acid sequences

| Collagen | Amino Acid Sequence |
|---|---|
| Gallus gallus | MAQLLRLFQTLLILLLRDYISAEDGETRASCRTAPADLVFILDGSYSVGPENFEIIKSWL |
| (chicken) type 21 | VNITRNFDIGPKFIQVGVVQYSDYPVLEIPLGTHESTENLIKEMESIHYLGGNTKTGRAI |
| alpha 1 collagen | QFAYDHLFAKSSRFLTKIAVVLTDGKSQDEVKDVAAEARKNKITLFAIGVGSEIEEDELK |
| | AIANKPSSTYVFYVEDYIAISRIKEVIKQKLCEESVCPTRIPVAARDEKGFDILVGLGVK |
| | KRVKKRIQIPTTNAKAYEVTSRVDLSELTRNVFPEGLPPSYVFVSTQRFKVKKTWDLWRV |
| | LSLDKRPQIAVTINGEEKTLSFTTTSLINGTQVITFAAPRVKTLFDEGWHQIRLLVTEDE |
| | VTLYIDDQEIETKPLHPVLGIYISGLTQIGKYSGKEETVOFDIQKLRIYCDPEQNNRETV |
| | CEIPGFNGECMNGPSDVGSTPAPCICPPGKOGPPGPKGDPGQPGNHGYPGQPGPDGKPGY |
| | QGSAGTPGIPGTPGVQGPRGLPGIKGEPGKDGTKGDRGLPGFPGLHGMPAPKGERGPKGD |
| | QGVPGIYGKKGSKGEKGDTGFPGMPGRSGDPGRSGKDGLPGSPGFKGEVGQPGSPGLEGH |
| | RGEPGIPGIPGNQGAKGQKGEIGPPGLPGAKGSPGETGLMGPEGSFGLPGAPGPKGDKGE |
| | PGLQGKPGSSGAKGEPGGPGAPGEPGYPGIPGTQGIKGDKGSQGESGIQGRKGEKGROGN |
| | PGLQGTEGLRGEQGEKGEKGDPGIRGINGQKGESGIQGLVGPPGVRGQPGDRGPPGPPGS |
| | DGKPAREFSEEFIRQVCSDVLRTQLPVILQSGRLONCNHCQSQSASPGLPGPPGPRGPEG |
| | PRGFPGLPGNDGVPGLTGIPGRPGARGTRGLPGKNGAKGNQGIGVPGIQGPPGPPGPEGP |
| | PGMSKEGRPGERGQPGKDGDRGSPGMPGPVGPPGICDPSLCFSVIVGRDPFRKGPNY |
| | (SEQ ID NO: 31) |
| Acipenser | MFSFVDSRTVLLLAAIQLCLLAVVKCODVEVQQPGRKGQKGEPGDITDVVGPRGPGGPMG |
| schrenckii | PPGEQGPRGERGDKGDKGGPGPRGRDGEPGTPGNPGPPGPPGPNGPPGLGGNFAAQMAGG |
| (Japanese | FDEKAGGAQMGVMQGPMGPMGPRGPPGPTGAPGPQGFQGNPGEPGEPGAAGPLGPRGPPG |
| sturgeon) type 2 | PSGKPGEDGEAGKPGKSGERGSPGPQGARGFPGTPGLPGIKGHRGYPGLDGAKGEAGAAG |
| alpha 1 collagen | SKGEAGSSGENGAPGPMGPRGLPGERGRNGPSGAAGARGNDGLPGPAGPPGPVGPAGAPG |
| | FPGSPGSKGEAGPTGARGPEGAQGPRGESGTPGSPGPSGASGNPGTDGIPGAKGSAGAPG |
| | IAGAPGFPGPRGPPGPQGATGPLGPKGQQGDPGIPGFKGEHGPKGEHGPAGPOGAPGPAG |
| | EEGKRGARGEPGAAGPLGPPGERGAPGNRGFPGQDGLAGPKGAPGERGQPGVGGPKGANG |
| | DPGRPGEPGLPGARGLTGRPGDAGPQGKGGPSGAAGEDGRPGPPGPQGARGQPGVMGFPG |
| | PKGANGEPGKAGEKGLVGPPGLRGLSGKDGETGAAGPPGPSGPAGERGEQGPPGPSGFQG |
| | LPGPPGPPGEGGKPGDQGVPGEAGAAGRAGPRGERGFPGERGSPGAQGLQGPRGLPGTPG |
| | TDGPKGATGPSGALGAQGPPGLQGMPGERGASGIAGAKGDRGDVGEKGPEGASGKDGSRG |
| | LTGPIGPPGPAGPNGEKGESGPSGPPGAAGTRGAPGDRGENGPPGPAGFAGPPGADGQPG |
| | AKGEQGEGGQKGDAGAPGPQGPSGAPGPQGPTGVSGPKGARGAQGPPGATGFPGAAGRVG |
| | PPGPNGNPGPSGPAGSAGKDGPKGVRGDAGPPGRAGDAGLQGAAGPPGEKGEPGEDGPPG |
| | PDGPSGPQGLGGNRGIVGLPGORGERGFPGLPGPSGEPGKQGAPGGAGDRGPPGPVGPPG |
| | LSGPSGEPGREGNPGSDGPPGRDGSAGIKGDRGQTGPAGAPGAPGAPGSPGPVGPTGKQG |
| | DRGESGAQGPAGPSGPAGARGMAGPQGPRGDKGEAGETGERGQKGHRGFTGLQGLPGPPG |
| | TAGDQGAAGPAGPTGARGPPGPVGPHGKDGSNGQPGPIGPPGPRGRSGEVGPAGPPGNAG |
| | PPGPPGPPGPGIDMSAFAGLAAPEKAPDPMRYMRADEASSSLROHDAEVDATLKSINNQI |
| | ENIRSPEGSKKNPARTCRDLKLCHPDWKSGDYWIDPNQGCAVDAIKVFCNMESGETCVYP |
| | NPASIPRKNWWTSKSADCKHVWFGETMNGGFHFSYGDDSLAPNTASIQMTFLRLLSTEAS |
| | QNLTYHCKNSIAYMDQSAGNLKKAVLLQGSNDVEIRAEGNSRFTYNVLEDGCTKHTDRWG |
| | KTVIEYKSQKTSRLPIVDIAPLDIGGSDQEFGVDIGPVCY |
| | (SEQ ID NO: 32) |

In some cases, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 31) from amino acid positions 1 to 537; from amino acid positions 1 to 542; from amino acid positions 1 to 547; from amino acid positions 1 to 552; from amino acid positions 1 to 557; from amino acid positions 1 to 562; from amino acid positions 1 to 567; from amino acid positions 1 to 572; or from amino acid positions 1 to 577. In some cases, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with a C-terminal truncation at any amino acid position (relative to SEQ ID NO: 31) from amino acid positions 726 to 957; from amino acid positions 731 to 957; from amino acid positions 736 to 957; from amino acid positions 741 to 957; from amino acid positions 746 to 957; from amino acid positions 751 to 957; from amino acid positions 756 to 957; from amino acid positions 761 to 957; from amino acid positions 766 to 957; from amino acid positions 769 to 957; from amino acid positions 774 to 957; from amino acid positions 779 to 957; or from amino acid positions 784 to 957. In some cases, a non-naturally occurring polypeptide as described herein (e.g., a truncated collagen) may comprise both an N-terminal truncation and a C-terminal truncation. For example, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 31) from amino acid positions 1 to 537; from amino acid positions 1 to 542; from amino acid positions 1 to 547; from amino acid positions 1 to 552; from amino acid positions 1 to 557; from amino acid positions 1 to 562; from amino acid positions 1 to 567; from amino acid positions 1 to 572; or from amino acid positions 1 to 577; and with a C-terminal truncation at any amino acid position (relative to SEQ ID NO: 31) from amino acid positions 726 to 957; from amino acid positions 731 to 957; from amino acid positions 736 to 957; from amino acid positions 741 to 957; from amino acid positions 746 to 957; from amino acid positions 751 to 957; from amino acid positions 756 to 957; from amino acid positions 761 to 957; from amino acid positions 766 to 957; from amino acid positions 769 to 957; from amino acid positions 774 to 957; from amino acid positions 779 to 957; or from amino acid positions 784 to 957. In a specific embodiment, a non-naturally occurring polypeptide (e.g., truncated collagen) disclosed herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at amino acid position 557 (relative to SEQ ID NO: 31); and with a C-terminal truncation at amino acid position 746 (relative to SEQ ID NO: 31). In another specific embodiment, a non-naturally occurring polypeptide (e.g., truncated collagen) disclosed herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at amino acid position 557 (relative to SEQ ID NO: 31); and with a C-terminal truncation at amino acid position 769 (relative to SEQ ID NO: 31).

In some cases, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 32) from amino acid positions 1 to 660; from amino acid positions 1 to 665; from amino acid positions 1 to 670; from amino acid positions 1 to 675; from amino acid positions 1 to 680; from amino acid positions 1 to 685; from amino acid positions 1 to 690; from amino acid positions 1 to 695; or from amino acid positions 1 to 700. In some cases, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with a C-terminal truncation at any amino acid position (relative to SEQ ID NO: 32) from amino acid positions 855 to 1420; from amino acid positions 860 to 1420; from amino acid positions 865 to 1420; from amino acid positions 870 to 1420; from amino acid positions 875 to 1420; from amino acid positions 880 to 1420; from amino acid positions 885 to 1420; from amino acid positions 890 to 1420; from amino acid positions 895 to 1420; or from amino acid positions 900 to 1420. In some cases, a non-naturally occurring polypeptide as described herein (e.g., a truncated collagen) may comprise both an N-terminal truncation and a C-terminal truncation. For example, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 32) from amino acid positions 1 to 660; from amino acid positions 1 to 665; from amino acid positions 1 to 670; from amino acid positions 1 to 675; from amino acid positions 1 to 680; from amino acid positions 1 to 685; from amino acid positions 1 to 690; from amino acid positions 1 to 695; or from amino acid positions 1 to 700; and with a C-terminal truncation at any amino acid position (relative to SEQ ID NO: 32) from amino acid positions 855 to 1420; from amino acid positions 860 to 1420; from amino acid positions 865 to 1420; from amino acid positions 870 to 1420; from amino acid positions 875 to 1420; from amino acid positions 880 to 1420; from amino acid positions 885 to 1420; from amino acid positions 890 to 1420; from amino acid positions 895 to 1420; or from amino acid positions 900 to 1420. In a specific embodiment, a non-naturally occurring polypeptide (e.g., truncated collagen) disclosed herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at amino acid position 680 (relative to SEQ ID NO: 32); and with a C-terminal truncation at amino acid position 880 (relative to SEQ ID NO: 32).

In some cases, a non-naturally occurring polypeptide (e.g., truncated collagen) may comprise any amino acid sequence provided herein. In some cases, a non-naturally occurring polypeptide (e.g., truncated collagen) may consist of any amino acid sequence provided herein. In some cases, a non-naturally occurring polypeptide (e.g., truncated collagen) may consist essentially of any amino acid sequence provided herein. In specific embodiments, the non-naturally occurring polypeptide has or comprises an amino acid sequence of any one of SEQ ID NO: 2, SEQ ID NO: 4, SEQ ID NO: 6, and SEQ ID NO: 8. In some embodiments, a non-naturally occurring polypeptide (e.g., truncated collagen) comprises an amino acid sequence having at least about 80%, at least about 85%, at least about 90%, at least about 95%, or at least about 98% sequence identity to any one of SEQ ID NO: 2, SEQ ID NO: 4, SEQ ID NO: 6, and SEQ ID NO: 8. In some embodiments, the non-naturally occurring polypeptide consists of or consists essentially of an amino acid sequence of any one of SEQ ID NO: 2, SEQ ID NO: 4, SEQ ID NO: 6, and SEQ ID NO: 8.

In some aspects, the non-naturally occurring polypeptide may include any chimeric collagen that includes at least one non-continuous collagen fragment. For example, the non-naturally occurring polypeptide can be a chimeric collagen in which a portion of N-terminus collagen is contiguous with a portion of C-terminus collagen where the portion of N-terminus collagen and the portion of C-terminus collagen are not contiguous in the natural or naturally-present corresponding collagens. In another example, the non-naturally occurring polypeptide can be a chimeric collagen in which a portion of C-terminus collagen is contiguous with a portion of N-terminus collagen (e.g., in a flipped or reverse order-C terminus collagen is located in the N-terminus of the portion of N-terminus collagen) where the portion of C-terminus collagen and the portion of N-terminus collagen are contiguous or non-contiguous in the natural or naturally-present corresponding collagens. In another example, the non-naturally occurring polypeptide can be a chimeric collagen in which one portion of a collagen polypeptide is contiguous with a portion of a second collagen polypeptide (e.g., a collagen molecule comprising a portion of a collagen from a first species contiguous with a portion of a collagen from a second species is a chimeric collagen, etc.).

Exemplary amino acid sequences of or nucleic acid sequences encoding the recombinant polypeptides are provided below:

A nucleotide sequence encoding a truncated collagen type 21 alpha 1
polypeptide from Gallus gallus (chicken)
SEQ ID NO: 1
GATACCGGTTTTCCGGGTATGCCTGGTCGTAGCGGTGATCCGGGTCGTAGCGGTAAAGATGGTCTGC
CTGGTAGCCCGGGTTTTAAAGGTGAAGTTGGTCAGCCAGGTAGCCCTGGTCTGGAAGGTCATCGTGG
TGAACCGGGTATTCCAGGTATTCCGGGTAATCAGGGTGCAAAAGGTCAGAAAGGCGAAATTGGTCCT
CCGGGTCTGCCAGGTGCCAAAGGTTCTCCGGGTGAAACCGGTCTGATGGGTCCTGAAGGTAGCTTTG
GCCTGCCTGGTGCACCGGGTCCGAAAGGTGACAAAGGTGAACCTGGTCTGCAGGGTAAACCGGGTAG
CAGCGGTGCAAAAGGCGAACCAGGTGGTCCGGGTGCTCCGGGTGAACCAGGCTATCCGGGTATTCCT
GGTACTCAGGGTATTAAAGGCGATAAAGGTAGCCAGGGTGAAAGCGGTATTCAGGGTCGTAAGGGTG
AAAAAGGCCGTCAGGGTAATCCAGGCCTGCAGGGCACCGAAGGTCTGCGTGGCGAACAGGGCGAAAA
AGGTGAGAAGGGTGACCCAGGCATTCGT
Amino acid sequence of a truncated collagen type 21 alpha 1 polypeptide
from Gallus gallus (chicken)
SEQ ID NO: 2
DTGFPGMPGRSGDPGRSGKDGLPGSPGFKGEVGQPGSPGLEGHRGEPGIPGIPGNQGAKGQKGEIGP
PGLPGAKGSPGETGLMGPEGSFGLPGAPGPKGDKGEPGLQGKPGSSGAKGEPGGPGAPGEPGYPGIP
GTQGIKGDKGSQGESGIQGRKGEKGROGNPGLQGTEGLRGEQGEKGEKGDPGIR
A nucleotide sequence encoding a truncated collagen type 21 alpha 1
polypeptide from Gallus gallus (chicken)
SEQ ID NO: 3
GATACTGGTTTCCCGGGGATGCCTGGGCGCTCAGGTGATCCGGGGCGTAGTGGAAAAG
ACGGTCTGCCGGGGTCCCCGGGCTTTAAGGGTGAGGTGGGTCAGCCCGGTAGTCCAGG
TTTAGAAGGTCACCGCGGAGAGCCCGGGATTCCAGGCATTCCTGGCAACCAGGGTGCC
AAGGGACAGAAAGGCGAAATTGGTCCGCCCGGCCTACCGGGCGCGAAAGGTTCTCCTG
GTGAAACCGGTCTCATGGGTCCGGAAGGTAGCTTCGGCCTGCCCGGCGCACCTGGTCCG
AAGGGCGATAAGGGGGAGCCTGGGCTGCAAGGTAAACCGGGTAGTTCTGGCGCCAAA
GGTGAACCCGGCGGTCCCGGTGCGCCAGGGGAACCAGGTTATCCTGGTATTCCTGGAA
CCCAAGGAATTAAAGGTGACAAAGGCTCACAGGGCGAAAGTGGTATACAGGGTCGCA
AGGGCGAAAAAGGACGTCAGGGCAATCCAGGCCTGCAGGGTACTGAAGGCCTGCGTG
GAGAACAGGGTGAGAAAGGTGAAAAAGGAGATCCTGGTATTCGC
Amino acid sequence of a truncated collagen type 21 alpha 1 polypeptide
from Gallus gallus (chicken)
SEQ ID NO: 4
DTGFPGMPGRSGDPGRSGKDGLPGSPGFKGEVGQPGSPGLEGHRGEPGIPGIPGNQGAKGQKGEIGP
PGLPGAKGSPGETGLMGPEGSFGLPGAPGPKGDKGEPGLQGKPGSSGAKGEPGGPGAPGEPGYPGIP
GTQGIKGDKGSQGESGIQGRKGEKGRQGNPGLQGTEGLRGEQGEKGEKGDPGIR
The nucleotide sequence encoding a truncated collagen type 21 alpha 1
polypeptide from Gallus gallus (chicken)
SEQ ID NO: 5
GATACTGGTTTCCCGGGGATGCCTGGGCGCTCAGGTGATCCGGGGCGTAGTGGAAAAGACGG
TCTGCCGGGGTCCCCGGGCTTTAAGGGTGAGGTGGGTCAGCCCGGTAGTCCAGGTTTAGAAGGTCAC
CGCGGAGAGCCCGGGATTCCAGGCATTCCTGGCAACCAGGGTGCCAAGGGACAGAAAGGCGAAATTG
GTCCGCCCGGCCTACCGGGCGCGAAAGGTTCTCCTGGTGAAACCGGTCTCATGGGTCCGGAAGGTAG
CTTCGGCCTGCCCGGCGCACCTGGTCCGAAGGGCGATAAGGGGGAGCCTGGGCTGCAAGGTAAACCG
GGTAGTTCTGGCGCCAAAGGTGAACCCGGCGGTCCCGGTGCGCCAGGGGAACCAGGTTATCCTGGTA
TTCCTGGAACCCAAGGAATTAAAGGTGACAAAGGCTCACAGGGCGAAAGTGGTATACAGGGTCGCAA
GGGCGAAAAAGGACGTCAGGGCAATCCAGGCCTGCAGGGTACTGAAGGCCTGCGTGGAGAACAGGGT
GAGAAAGGTGAAAAAGGAGATCCTGGTATTCGCGGCATTAACGGTCAAAAGGGTGAAAGTGGGATAC
AAGGTCTTGTCGGTCCGCCCGGAGTTAGAGGCCAG
Amino acid sequence of a truncated collagen type 21 alpha 1 polypeptide
from Gallus gallus (chicken)
SEQ ID NO: 6
DTGFPGMPGRSGDPGRSGKDGLPGSPGFKGEVGQPGSPGLEGHRGEPGIPGIPGNQGAKGQKGEIGP
PGLPGAKGSPGETGLMGPEGSFGLPGAPGPKGDKGEPGLQGKPGSSGAKGEPGGPGAPGEPGYPGIP
GTQGIKGDKGSQGESGIQGRKGEKGROGNPGLQGTEGLRGEQGEKGEKGDPGIRGINGQKGESGIQG
LVGPPGVRGQ
The nucleotide sequence encoding a truncated collagen type 2 alpha 1
polypeptide from Acipenser schrenckii (Japanese sturgeon)
SEQ ID NO: 7
GTCTGCAGGGTATGCCTGGTGAACGTGGTGCAAGCGGTATTGCCGGTGCAAAAGGTGATCGTGGTGA
TGTTGGTGAAAAAGGTCCGGAAGGTGCCAGCGGTAAAGATGGTAGCCGTGGTCTGACCGGTCCGATT
GGTCCGCCTGGTCCGGCAGGTCCGAATGGCGAAAAAGGTGAAAGCGGTCCGAGCGGTCCTCCGGGTG
CAGCAGGTACTCGTGGTGCACCGGGTGATCGCGGTGAAAATGGTCCACCGGGTCCTGCCGGTTTTGC
AGGTCCGCCAGGTGCAGATGGTCAGCCTGGTGCCAAAGGCGAACAAGGCGAAGGTGGTCAGAAAGGT
GATGCAGGCGCTCCGGGTCCGCAGGGTCCTTCTGGTGCACCTGGTCCTCAGGGTCCGACCGGTGTTT
CTGGTCCGAAAGGCGCACGTGGTGCCCAGGGTCCACCTGGTGCGACCGGTTTTCCTGGCGCAGCAGG
TCGTGTTGGTCCTCCAGGTCCTAATGGTAATCCGGGTCCAAGCGGTCCTGCAGGTAGCGCAGGCAAA
GATGGTCCTAAAGGTGTACGCGGTGATGCTGGTCCTCCTGGCCGTGCCGGTGATGCCGGT
Amino acid sequence of a truncated collagen type 2 alpha 1 polypeptide
from Acipenser schrenckii (Japanese sturgeon)
SEQ ID NO: 8
GLQGMPGERGASGIAGAKGDRGDVGEKGPEGASGKDGSRGLTGPIGPPGPAGPNGEKGESGPSGPPG
AAGTRGAPGDRGENGPPGPAGFAGPPGADGQPGAKGEQGEGGQKGDAGAPGPQGPSGAPGPQGPTGV
SGPKGARGAQGPPGATGFPGAAGRVGPPGPNGNPGPSGPAGSAGKDGPKGVRGDAGPPGRAGDAG
The nucleotide sequence encoding a secretion signal sequence named
Secretion Signal Sequence 1
SEQ ID NO: 9
ATGAAAAAGATTTGGCTGGCGCTGGCTGGTTTAGTTTTAGCGTTTAGCGCATCGGCG
Amino acid sequence of a Secretion Signal Sequence 1
SEQ ID NO: 10
MKKIWLALAGLVLAFSASA
The nucleotide sequence encoding a secretion signal sequence named
Secretion Signal Sequence 2
SEQ ID NO: 11
ATGAAAAAAGGTTTCATGCTGTTCACCCTCCTCGCTGCGTTCTCTGGTTTCGCGCAGGCT
Amino acid sequence of a Secretion Signal Sequence 2
SEQ ID NO: 12
MKKGFMLFTLLAAFSGFAQA
The nucleotide sequence encoding a secretion signal sequence named
Secretion Signal Sequence 3
SEQ ID NO: 13
ATGATGATCACCCTGCGTAAACTGCCGCTGGCTGTTGCTGTTGCTGCTGGTGTTATGTCTGCTCAGG
CTATGGCT
Amino acid sequence of a Secretion Signal Sequence 3
SEQ ID NO: 14
MMITLRKLPLAVAVAAGVMSAQAMA
The nucleotide sequence encoding a secretion signal sequence named
Secretion Signal Sequence 4
SEQ ID NO: 15
ATGAAAAAAACCGCTATCGCTATCGCTGTTGCTCTGGCTGGTTTCGCTACCGTTGCTCAGGCT
Amino acid sequence of a Secretion Signal Sequence 4
SEQ ID NO: 16
MKKTAIAIAVALAGFATVAQA
The nucleotide sequence encoding a secretion signal sequence named
Secretion Signal Sequence 5
SEQ ID NO: 17
ATGAAAGTTAAAGTTCTGTCTCTGCTGGTTCCGGCTCTGCTGGTTGCTGGTGCTGCTAACGCT
Amino acid sequence of a Secretion Signal Sequence 5
SEQ ID NO: 18
MKVKVLSLLVPALLVAGAANA
The nucleotide sequence encoding a secretion signal sequence named
Secretion Signal Sequence 6
SEQ ID NO: 19
ATGAAAAAAAACATCCTGTCTCTGTCTATGGTTGCTCTGTCTCTGTCTCTGGCTCTGGGTTCTGTTT
CTGTTACCGCT
Amino acid sequence of a Secretion Signal Sequence 6
SEQ ID NO: 20
MKKNILSLSMVALSLSLALGSVSVTA
The nucleotide sequence encoding a secretion signal sequence named
Secretion Signal Sequence 7
SEQ ID NO: 21
ATGCTGAACCCGAAAGTTGCTTACATGGTTTGGATGACCTGCCTGGGTCTGACCCTGCCGTCTCAGG
CT
Amino acid sequence of a Secretion Signal Sequence 7
SEQ ID NO: 22
MLNPKVAYMVWMTCLGLTLPSQA
The nucleotide sequence encoding a secretion signal sequence named
Secretion Signal Sequence 8
SEQ ID NO: 23
ATGAAACAGGCTCTGCGTGTAGCGTTCGGTTTCCTGATACTGTGGGCTTCTGTTCTGCACGCT
Amino acid sequence of a Secretion Signal Sequence 8
SEQ ID NO: 24
MKQALRVAFGFLILWASVLHA
A codon-optimized nucleotide sequence encoding a truncated collagen type
2 alpha 1 polypeptide from Acipenser schrenckii (Japanese sturgeon)
SEQ ID NO: 25
GGTCTGCAGGGTATGCCGGGTGAACGTGGTGCCAGCGGTATTGCAGGTGCCAAAGGTGATCGTGGTG
ATGTTGGTGAAAAAGGTCCGGAAGGTGCAAGCGGTAAAGATGGTAGCCGTGGTCTGACCGGTCCGAT
TGGTCCGCCGGGTCCGGCCGGTCCGAATGGTGAAAAAGGTGAAAGCGGTCCGAGCGGTCCGCCGGGT
GCAGCCGGTACCCGTGGTGCACCGGGTGATCGTGGTGAAAATGGTCCGCCGGGTCCGGCCGGTTTTG
CAGGTCCGCCGGGTGCCGATGGTCAGCCGGGTGCAAAAGGTGAACAGGGTGAAGGTGGTCAGAAAGG
TGATGCCGGTGCACCGGGTCCGCAGGGTCCGAGCGGTGCCCCGGGTCCGCAGGGTCCGACCGGTGTT
AGCGGTCCGAAAGGTGCACGTGGTGCCCAGGGTCCGCCGGGTGCAACCGGTTTTCCGGGTGCCGCAG
GTCGTGTTGGTCCGCCGGGTCCGAATGGTAATCCGGGTCCGAGCGGTCCGGCAGGTAGCGCCGGTAA
AGATGGTCCGAAAGGTGTTCGTGGTGATGCAGGTCCGCCGGGTCGTGCCGGTGATGCAGGTTAA
A codon-optimized nucleotide sequence encoding a truncated collagen type
2 alpha 1 polypeptide from Acipenser schrenckii (Japanese sturgeon)
SEQ ID NO: 26
GGCCTGCAAGGCATGCCAGGCGAGCGCGGCGCGTCTGGCATCGCGGGCGCGAAGGGCGACCGCGGCG
ACGTGGGCGAGAAGGGCCCTGAGGGCGCGTCCGGCAAGGACGGCTCTCGCGGCCTGACAGGCCCAAT
CGGCCCTCCAGGCCCTGCGGGCCCAAACGGCGAGAAGGGCGAGTCCGGCCCTTCTGGCCCACCTGGC
GCGGCGGGCACACGCGGCGCGCCAGGCGACCGCGGCGAGAACGGCCCTCCAGGCCCTGCGGGCTTCG
CGGGCCCACCTGGCGCGGACGGCCAACCAGGCGCGAAGGGCGAGCAAGGCGAGGGCGGCCAAAAGGG
CGACGCGGGCGCGCCTGGCCCACAAGGCCCTTCTGGCGCGCCAGGCCCTCAAGGCCCAACAGGCGTG
TCCGGCCCTAAGGGCGCGCGCGGCGCGCAAGGCCCACCTGGCGCGACAGGCTTCCCAGGCGCGGCGG
GCCGCGTGGGCCCTCCAGGCCCTAACGGCAACCCAGGCCCTTCTGGCCCAGCGGGCTCCGCGGGCAA
GGACGGCCCTAAGGGCGTGCGCGGCGACGCGGGCCCACCTGGCCGCGCGGGCGACGCGGGCTGA
A codon-optimized nucleotide sequence encoding a truncated collagen type
2 alpha 1 polypeptide from Acipenser schrenckii (Japanese sturgeon)
SEQ ID NO: 27
GGTTTGCAAGGTATGCCAGGGGAACGGGGTGCGTCCGGGATAGCCGGGGCAAAAGGTGATCGAGGCG
ATGTAGGAGAAAAAGGCCCAGAAGGGGCGTCAGGTAAGGACGGATCTCGCGGCTTGACGGGACCTAT
CGGGCCTCCAGGTCCCGCCGGCCCTAATGGGGAAAAAGGCGAGAGTGGGCCGTCTGGTCCGCCCGGC
GCCGCTGGCACACGTGGAGCGCCGGGCGATCGTGGTGAGAACGGACCACCGGGTCCTGCTGGTTTTG
CGGGACCTCCGGGAGCAGACGGCCAGCCGGGCGCTAAAGGTGAACAGGGTGAAGGTGGCCAAAAAGG
CGATGCAGGCGCACCGGGTCCGCAGGGCCCTTCAGGTGCACCGGGTCCACAGGGCCCAACTGGCGTT
TCAGGGCCGAAAGGCGCAAGAGGTGCTCAGGGTCCGCCCGGGGCAACTGGGTTTCCTGGAGCGGCCG
GCCGTGTTGGACCTCCGGGGCCGAACGGAAACCCTGGACCGTCTGGACCAGCCGGTTCAGCGGGTAA
GGATGGTCCTAAGGGTGTAAGGGGTGACGCAGGTCCCCCTGGACGTGCAGGGGATGCGGGGTAG
A codon-optimized nucleotide sequence encoding a truncated collagen type
2 alpha 1 polypeptide from Acipenser schrenckii (Japanese sturgeon)
SEQ ID NO: 28
GGGTTACAAGGTATGCCGGGAGAACGTGGAGCGTCAGGAATTGCTGGGGCCAAAGGTGATCGTGGTG
ATGTTGGCGAGAAAGGGCCCGAAGGCGCATCTGGTAAAGATGGCTCACGCGGGTTAACTGGACCAAT
CGGACCACCAGGCCCCGCTGGGCCTAATGGTGAAAAGGGTGAAAGTGGCCCTTCTGGACCCCCAGGA
GCCGCCGGTACACGTGGAGCGCCAGGCGATCGTGGCGAAAACGGACCGCCCGGACCTGCAGGTTTTG
CGGGACCCCCTGGAGCAGACGGCCAACCAGGAGCAAAAGGTGAGCAAGGTGAAGGTGGACAAAAGGG
AGATGCCGGAGCGCCAGGCCCCCAAGGCCCATCAGGAGCTCCAGGACCTCAAGGTCCAACTGGTGTA
TCAGGGCCTAAGGGTGCGCGCGGCGCTCAAGGACCGCCTGGCGCAACTGGCTTTCCGGGAGCTGCTG
GTCGTGTGGGCCCGCCTGGCCCAAACGGAAATCCAGGCCCTTCAGGCCCGGCGGGCTCAGCCGGAAA
AGACGGTCCGAAGGGAGTCCGTGGAGATGCGGGACCGCCAGGACGCGCTGGCGATGCAGGCTAA
A codon-optimized nucleotide sequence encoding a truncated collagen type
2 alpha 1 polypeptide from Acipenser schrenckii (Japanese sturgeon)
SEQ ID NO: 29
GGTTTACAGGGAATGCCAGGGGAACGCGGCGCCTCAGGGATTGCCGGTGCTAAAGGAGATCGTGGCG
ACGTGGGTGAAAAGGGTCCCGAGGGAGCATCAGGTAAGGATGGTTCCCGTGGTTTGACGGGACCTAT
TGGACCTCCGGGTCCTGCAGGTCCGAACGGCGAAAAGGGGGAAAGCGGGCCTAGTGGTCCACCCGGC
GCCGCAGGTACCCGTGGTGCCCCAGGCGACCGCGGGGAGAATGGACCGCCTGGCCCTGCCGGTTTTG
CGGGTCCTCCAGGAGCCGATGGGCAGCCCGGTGCAAAAGGAGAGCAGGGAGAGGGAGGTCAAAAGGG
AGATGCCGGCGCCCCGGGCCCTCAGGGACCAAGCGGTGCGCCAGGCCCCCAGGGTCCTACGGGTGTT
AGCGGGCCGAAAGGCGCACGCGGAGCGCAGGGCCCACCTGGTGCAACAGGCTTCCCAGGAGCTGCGG
GGCGCGTCGGACCTCCGGGACCCAATGGAAACCCAGGTCCGTCAGGGCCGGCAGGCTCCGCAGGGAA
AGATGGTCCCAAAGGCGTGCGTGGAGACGCAGGGCCCCCCGGACGCGCCGGCGATGCGGGATAA
A codon-optimized nucleotide sequence encoding a truncated collagen type
21 polypeptide from Gallus gallus
SEQ ID NO: 30
GATACTGGTTTCCCGGGGATGCCTGGGCGCTCAGGTGATCCGGGGCGTAGTGGAAAAGACGGTCTGC
CGGGGTCCCCGGGCTTTAAGGGTGAGGTGGGTCAGCCCGGTAGTCCAGGTTTAGAAGGTCACCGCGG
AGAGCCCGGGATTCCAGGCATTCCTGGCAACCAGGGTGCCAAGGGACAGAAAGGCGAAATTGGTCCG
CCCGGCCTACCGGGCGCGAAAGGTTCTCCTGGTGAAACCGGTCTCATGGGTCCGGAAGGTAGCTTCG
GCCTGCCCGGCGCACCTGGTCCGAAGGGCGATAAGGGGGAGCCTGGGCTGCAAGGTAAACCGGGTAG
TTCTGGCGCCAAAGGTGAACCCGGCGGTCCCGGTGCGCCAGGGGAACCAGGTTATCCTGGTATTCCT
GGAACCCAAGGAATTAAAGGTGACAAAGGCTCACAGGGCGAAAGTGGTATACAGGGTCGCAAGGGCG
AAAAAGGACGTCAGGGCAATCCAGGCCTGCAGGGTACTGAAGGCCTGCGTGGAGAACAGGGTGAGAA
AGGTGAAAAAGGAGATCCTGGTATTCGC

In some embodiments, the non-naturally occurring polypeptide comprises an amino acid sequence of any one of SEQ ID NOs: 2, 4, 6, and 8. In some embodiments, the non-naturally occurring polypeptide comprises an amino acid sequence having a sequence identity of at least about 80%, at least about 85%, at least about 90%, at least about 95%, or at least about 98% thereof, or the like, to the amino acid sequence of any one of SEQ ID NOs: 2, 4, 6, and 8. Alternatively and/or additionally, the non-naturally occurring polypeptide is encoded by a nucleic acid sequence of any one of SEQ ID NOs: 1, 3, 5, 7, and 25-30. In some embodiments, the non-naturally occurring polypeptide is encoded by a nucleic acid having sequence identity of at least about 65%, at least about 70%, at least about 75%, at least about 80%, at least about 85%, at least about 90%, at least about 95%, or at least about 98% thereof, or the like, to the nucleic acid sequence of any one of SEQ ID NOs: 1, 3, 5, 7, and 25-30.

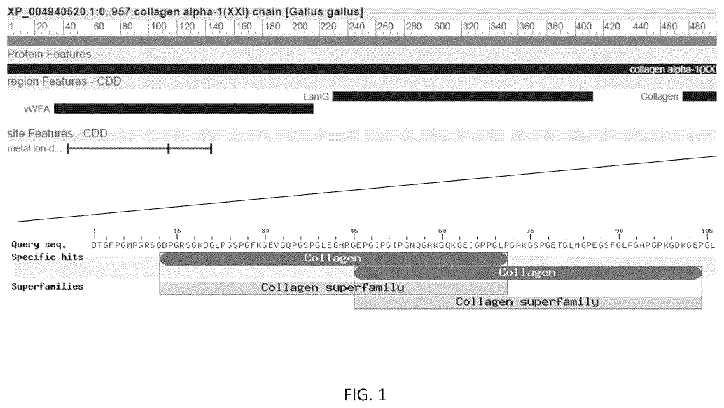

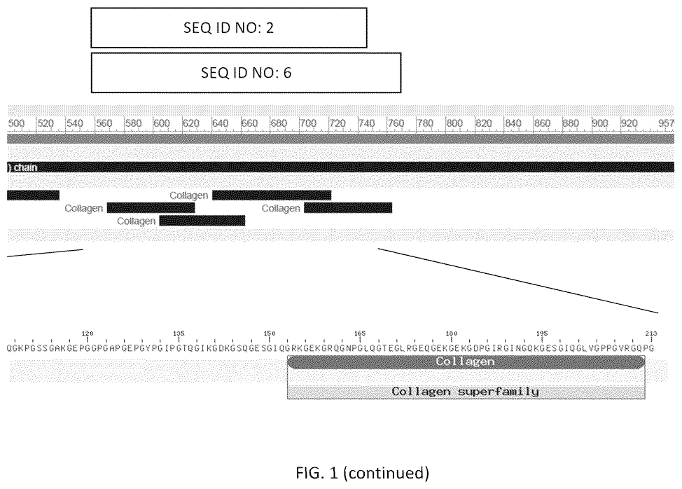

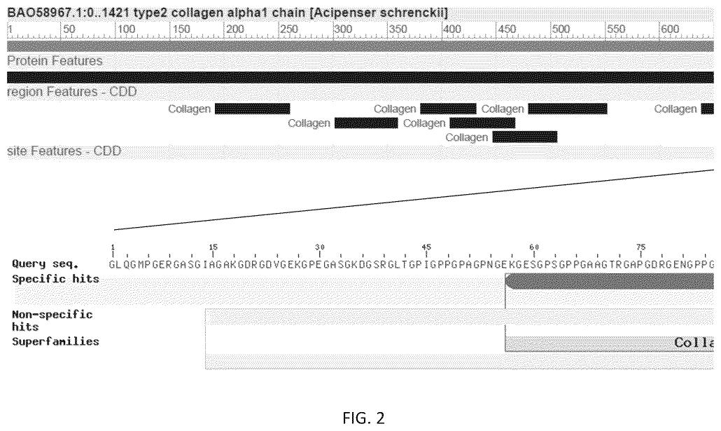

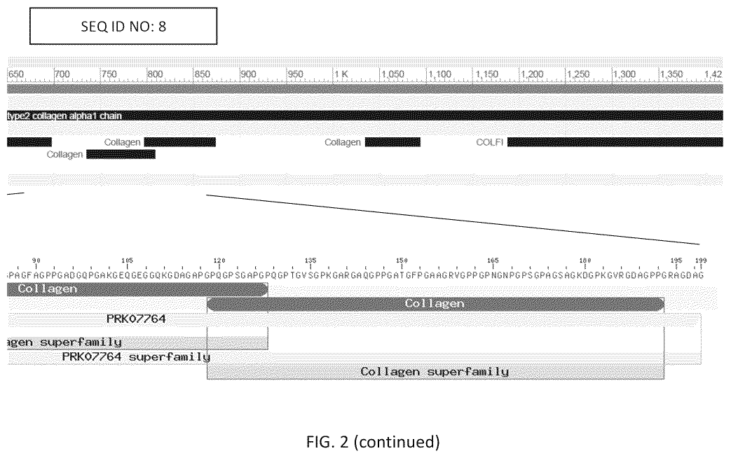

In some aspects, the non-naturally occurring polypeptides provided herein may or may not contain one or more domains from natural collagen. FIG. 1 and FIG. 2 depict alignments of exemplary non-naturally occurring polypeptides (e.g., truncated collagens) of the disclosure with the corresponding naturally occurring collagen. FIG. 1 depicts an alignment of a non-naturally occurring polypeptide of SEQ ID NO: 2 and SEQ ID NO: 6 with Gallus gallus type 21 alpha 1 collagen (e.g., SEQ ID NO: 31). FIG. 2 depicts an alignment of a non-naturally occurring polypeptide of SEQ ID NO: 8 with Acipenser schrenckii type 2 alpha 1 collagen. FIG. 1 and FIG. 2 demonstrate that non-naturally occurring polypeptides may have one or more domains found in natural collagen (e.g., collagen triple helix repeat domains). FIG. 1 and FIG. 2 further demonstrate that non-naturally occurring polypeptides may lack one or more domains found in natural collagen (e.g., Von Willebrand factor type A (vWA) domain, laminin G domain, fibrillar collagen C-terminal domain). In some aspects, a non-naturally occurring polypeptide provided herein may contain one or more collagen triple helix repeat domains. In some aspects, a non-naturally occurring polypeptide provided herein may lack one or more of a Von Willebrand factor type A (vWA) domain, a laminin G domain, and a fibrillar collagen C-terminal domain).

In some embodiments, the non-naturally occurring polypeptide (e.g., recombinant polypeptide) includes a secretion signal sequence. Any suitable secretion signal sequence (e.g., hydrophobic signaling peptides, Sec signal peptides, Tat signal peptides, etc.) that can induce the non-naturally occurring polypeptide (e.g., recombinant polypeptide) to be secreted to the periplasmic and/or extracellular space (e.g., when produced in a recombinant host cell). Exemplary secretion signal sequences include a peptide having an amino acid sequence of any one of SEQ ID NOs: 10, 12, 14, 16, 18, 20, 22, and 24. Alternatively and/or additionally, the secretion signal sequence includes a peptide encoded by a nucleic acid sequence of any one of SEQ ID NOs: 9, 11, 13, 15, 17, 19, 21, and 23. The secretion signal sequence is preferably located at the N-terminus of the non-naturally occurring polypeptide (e.g., recombinant polypeptide). Yet, it is contemplated that the secretion signal sequence can be located at other than N-terminus where the secretion signal sequence remains functional.

The non-naturally occurring polypeptide (e.g., recombinant polypeptide) as described herein can be expressed or generated via a nucleic acid sequence encoding the non-naturally occurring polypeptide (e.g., recombinant polypeptide). Thus, another aspect of the disclosure includes an expression vector comprising a nucleic acid sequence encoding the non-naturally occurring polypeptide (e.g., recombinant polypeptide). In some embodiments, the expression vector is a bacterial expression vector. In some embodiments, the expression vector is a yeast expression vector. In some embodiments, the expression vector is an insect expression vector. Any suitable expression vector that can induce the protein expression from the inserted nucleic acid encoding the non-naturally occurring polypeptide (e.g., recombinant polypeptide). Exemplary bacterial expression vectors may include pGEX vectors where glutathione S-transferase is used as a fusion partner and gene expression is under the control of the tac promoter, or pET vectors (e.g., pET28 vector, etc.) which uses a T7 promoter. Exemplary yeast expression vectors may include pPIC vectors, which uses the AOX1 promoter inducible with methanol. In some embodiments, the expression vector is in a plasmid form (e.g., including bacterial artificial chromosome form, etc.) that are independently present in the host cell (e.g., cells expressing the recombinant polypeptide). In some embodiments, the expression vector is stably integrated into the chromosome of the host cell via random or targeted integration.

In some embodiments, the nucleic acid sequence encoding the non-naturally occurring polypeptide (e.g., recombinant polypeptide) is codon-optimized to be expressed in non-animal cells, preferably in bacterial cells. As used herein, “codon-optimized” means that the codon composition is improved for expression in the heterologous cells (e.g., microbial cells, bacterial cells, etc.) without altering the encoded amino acid sequences. Non-limiting examples of codon-optimized nucleic acid sequences (e.g., encoding a non-naturally occurring polypeptide as described herein) include SEQ ID NOs: 25-30.

In some embodiments, the expression vector may include one or more selection agent. The selection agents include certain sugars including galactose containing sugars or antibiotics including ampicillin, hygromycin, G418 and others. Enzymes that are used to confer resistance to the selection agent include β-galactosidase or a β-lactamase. Alternatively and/or additionally, the expression vector includes an inducible promoter or a constitutive promoter (e.g., CMV promoter, etc.) such that the nucleic acid encoding the recombinant protein is operatively linked to the inducible promoter or the constitutive promoter. For example, the expression vector may include tetracycline-inducible promoter pTET, araC-ParaBAD inducible promoter, or IPTG inducible lac promoter. As used herein, “operatively linked” promoter and nucleic acid means that the expression of the nucleic acid (e.g., transcription, translation, etc.) is at least under partial control of the promoter.

In some embodiments, the nucleic acid encoding the non-naturally occurring polypeptide (e.g., recombinant polypeptide) (e.g., a nucleic acid of any one of SEQ ID NOs: 1, 3, 5, 7, and 25-30), and the expression vector may have an overlap of from 20 to 50 bp long, from 20 to 40 bp long, from 20 to 30 bp long, or from 30 to 40 bp long. Such overlap can be added using PCR with a DNA polymerase (e.g., PRIMESTAR® GXL polymerase (takarabio.com/products/pcr/gc-rich-pcr/primestar-gxl-dna-polymerase)). Opened expression vector and the insert nucleic acid encoding the non-naturally occurring polypeptide (e.g., recombinant polypeptide) can be assembled together into the final plasmid using any suitable cloning system (e.g., IN-FUSION® Cloning (takarabio.com/products/cloning/in-fusion-cloning) or SGI Gibson assembly (us.vwr.com/store/product/17613857/gibson-assembly-hifi-1-step-kit-synthetic-genomics-inc)).

Such prepared expression vector (or plasmid) can be used to generate genetically engineered or modified organisms, or a recombinant cell to produce the non-naturally occurring polypeptides described herein (e.g., collagens, truncated collagens, or collagen fragments). Preferably, the recombinant cells contain at least one copy of a plasmid or a stably integrated heterologous nucleic acid sequence encoding the non-naturally occurring polypeptide (e.g., collagens, truncated collagens, or collagen fragments, preferably collagens, truncated collagens, or collagen fragments of, or derived from, Gallus gallus collagen and/or Acipenser schrenckii collagen). In some embodiments, the recombinant cell is a microbial cell. For example, where the expression vector is bacterial expression vector, the expression vector can be inserted into (e.g., via any suitable transformation method) the bacterial cells for protein expression (e.g., Escherichia coli including BL-21 cells, etc.) to be independently present in the cytoplasm of the bacteria (e.g., as a plasmid form) or to be at least temporarily and/or stably integrated into the bacterial chromosome.

Consequently, the transformed cells can be cultivated in a suitable media. Preferably, the suitable media includes a minimal media and the cells are frozen in 1.5 aliquots with vegetable glycerin at a ratio of 50:50 of cells of cells to glycerin. For protein expression, one vial of the frozen cultured cells can be cultured in a suitable amount of bacteria culture media (e.g., minimal media, 50 mL, 100 mL, etc.) for at least 6 hours, at least 8 hours, at least 10 hours, at least 12 hours, at least overnight at least 36° C., preferably at about 37° C. by continuously shaking the culture (e.g., at least 100 rpm, at least 200 rpm, at least 250 rpm, etc.). Table 2 and Table 3 show the exemplary formulation of the minimal media that can be used for cell cultivation and culture.

TABLE 2
Minimal Media Formulation

1) Autoclave 5 L of 550 g/kg Glucose syrup at concentration
in DI water. (VWR, product #97061-170).

| | |
|---|---|
| 2) Autoclave in 3946 mL | 20 g (NH₄)₂HPO₄. |
| of DI water and add | (VWR, product # 97061-932). |
| | 66.5 g KH₂PO₄. |
| | (VWR, product # 97062-348). |
| | 22.5 g H₃C₆H₅O₇. |
| | (VWR, product #BDH9228-2.5 KG). |
| | 8.85 g MgSO₄•7H₂O. |
| | (VWR, product # 97062-134). |
| | 10 mL of 1000x Trace metals formulation |

After autoclaving, add:
118 g of (1) to (2)
5 mL of 25 mg/mL Kanamycin Sulfate (VWR-V0408)
Use 28% NH₄OH (VWR, product #BDH3022) to adjust pH to 6.1.

TABLE 3
Trace metals formulation

| | |
|---|---|
| Ferrous Sulfate Heptahydrate | 27.8 g/L (Spectrum, 7782-63-0) |
| Zinc Sulfate heptahydrate | 2.88 g/L (Spectrum, 7446-20-0) |
| Calcium chloride dihydrate | 2.94 g/L (Spectrum, 2971347) |
| Sodium molybdate dihydrate | 0.48 g/L (Spectrum, 10102-40-6) |
| Manganese chloride tetrahydrate | 1.26 g/L (Spectrum, 13446-34-9) |
| Sodium selenite | 0.35 g/L (Spectrum, 10102-18-8) |
| Boric acid | 0.12 g/L (Spectrum, 10043-35-3) |

In some embodiments, transformed cells can then be transferred to a larger volume of growth media (e.g., minimal media) and grown for at least 4 hours, at least 5 hours, at least 6 hours, at least 7 hours, at least 8 hours, from 5 to 10 hours, from 5 to 9 hours, from 6 to 9 hours, and/or alternatively until the cell density in the media reaches optical density (OD) of 600.

Additionally, fermentation process can be performed at various temperature ranging from 22° C. to 33° C., from 29° C. to 33° C., from 30° C. to 32° C., from 23° C. to 29° C., or from 25° C. to 28° C. In some embodiments, the temperature of the fermentation can be maintained at a constant temperature and immediately upon completion of fermentation the non-naturally occurring polypeptide can be purified. Alternatively, the temperature of the fermentations can be maintained for a desired period of time and when cell densities of OD600 of 10-20 are reached, then the temperature can be reduced to induce protein production. In such embodiments, typically, the temperature is reduced from 28° C. to 25° C. During the fermentation, protein expression in the bacteria can be induced by adding induction reagent. For example, where the expression vector contains lac promoter and the nucleic acid encoding the non-naturally occurring polypeptide (e.g., truncated collagen, collagen fragments, or collagen) is under the control of the lac promoter, the expression of the nucleic acid can be induced by adding isopropyl β-d-1-thiogalactopyranoside (IPTG) at a concentration ranging from 0.1-1.5 mM, from 0.1-1.0 mM, or from 0.1-0.5 mM. Fermentation can be continued for 20-24 hours, or in some embodiments, for 40-60 hours.

It is contemplated that such generated recombinant cells (e.g., recombinant bacteria transformed with the expression vector) intracellularly express the non-naturally occurring polypeptides (e.g., truncated collagen, collagen fragments, or collagen) encoded by the nucleic acids in the expression vector. Such intracellularly expressed polypeptides (e.g., truncated collagen, collagen fragments, or collagen) can then be secreted (via a secretion signal sequence) to the extracellular space (e.g., into a culture media). Thus, in some embodiments, the culture media can contain secreted recombinant protein (e.g., truncated collagen, collagen fragments, or collagen) encoded by the nucleic acids.

Thus, another aspect of the disclosure includes a composition including the non-naturally occurring polypeptide (e.g., recombinant collagen, truncated collagen, collagen fragments, or collagen) encoded by the nucleic acids. In some embodiments, the composition may include the recombinant cell comprising an integrated heterologous nucleic acid sequence encoding a non-naturally occurring polypeptide (e.g., collagen, a truncated collagen, or fragment thereof), and/or the culture medium (e.g., growth media, cultivation media, etc.) for the recombinant cell.

Alternatively and/or additionally, the composition may include purified recombinant polypeptides from the recombinant cells and/or the culture medium. In some embodiments, the recombinant polypeptides are purified from the culture medium where the recombinant cells grow and secrete the recombinant polypeptides thereto. In some embodiments, the recombinant polypeptide is coupled with a tag (e.g., histidine tag, etc) such that the recombinant polypeptide can be purified using affinity purification is known as immobilized metal affinity chromatography (IMAC). Alternatively, the recombinant polypeptide can be purified via column chromatography. For example, the recombinant polypeptide can be purified by acid treatment of homogenized growth media. In such example, the pH of the growth media (e.g., fermentation broth) can be decreased to from 3 to 3.5 using 5-50% sulfuric acid. The recombinant cells are then separated using centrifugation. Supernatant of the acidified broth can be tested on a polyacrylamide gel and determined whether it contains the recombinant polypeptide in relatively high abundance compared to starting pellet. The recombinant polypeptide slurry obtained is generally high in salts. To obtain volume and salt reduction, concentration and diafiltration steps can be performed using filtration steps. For example, the filtration step can be performed using EMD Millipore Tangential Flow Filtration system with ultrafiltration cassettes of 0.1 m² each. Total area of filtration in this example can be 0.2 m² using two cassettes in parallel. A volume reduction of 5× and a salt reduction of 19× can be achieved in the TFF stage. Final slurry can be run on an SDS-PAGE gel to confirm presence of the recombinant polypeptide. The purified recombinant polypeptide can then be analyzed on an SDS-PAGE gel to identify a corresponding thick and clear band observed at the expected sizes for each respective polypeptide. Quantification of titers and purity can be further conducted using reverse phase and size exclusion HPLC chromatography. It is preferred that the purity of the purified recombinant polypeptides is at least at least about 80%, at least about 85%, at least about 90%, at least about 95%, or at least about 99%.

In various aspects, provided herein are compositions and formulations comprising a polypeptide of the disclosure and one or more additional ingredients. The compositions and formulations of the present disclosure can include or be incorporated into all types of vehicles and carriers. The vehicle or carrier can be a cosmetically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in compositions and formulations of the present disclosure. In certain aspects, the concentrations and combinations of the compounds, ingredients, and agents can be selected in such a way that the combinations are chemically compatible and do not form complexes which precipitate from the finished product.

In some aspects, the compositions and formulations of the present disclosure can further include a surfactant, a silicone containing compound, a UV agent, an oil, and/or other ingredients identified in this specification or those known in the art. The composition can be a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, gel serums, gel emulsions, etc. The composition can be formulated for topical skin application at least 1, at least 2, at least 3, at least 4, at least 5, at least 6, at least 7, or more times a day during use.

The compositions and formulations of the present disclosure can include a triglyceride. Non-limiting examples include small, medium, and large chain triglycerides. The compositions and formulations of the present disclosure can also include preservatives. Non-limiting examples of preservatives include phenoxyethanol, methylparaben, propylparaben, iodopropynyl butylcarbamate, potassium sorbate, sodium benzoate, or any mixture thereof. In some embodiments, the compositions and formulations of the disclosure are paraben-free.

Compositions and formulations of the present disclosure can have UVA and UVB absorption properties. The compositions and formulations of the present disclosure can have a sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions and formulations of the present disclosure can be sunscreen lotions, sprays, or creams.

The compositions and formulations of the present disclosure can also include any one of, any combination of, or all of the following additional ingredients: a conditioning agent, a moisturizing agent, a pH adjuster, a structuring agent, inorganic salts, a preservative, a thickening agent, a silicone containing compound, an essential oil, a fragrance, a vitamin, a pharmaceutical ingredient, or an antioxidant, or any combination of such ingredients or mixtures of such ingredients. The amounts of such ingredients can range from 0.0001% to 99.9% by weight or volume of the composition, or any integer or range in between.

The CTFA International Cosmetic Ingredient Dictionary and Handbook (2004 and 2008) describes a wide variety of non-limiting cosmetic ingredients that can be used in the context of the present disclosure. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, gluconolactone, calcium gluconate, cyclohexasiloxane, and dipotassium glycyrrhizate).

In some embodiments, the compositions, formulations, and/or personal care products of the present disclosure include one or more additional ingredients selected from the group consisting of: levulinic acid, polyglyceryl-3 methylglucose distearate, glyceryl undecylenate, Simmondsia chinensis (Jojoba) seed oil, polyacrylate cross-polymer, squalane, sodium hyaluronate, acrylic acid polymers (carbomers), pentylene glycol, sodium lauryl sulfoacetate, sodium oleoyl sarcosinate, sodium oleate, Ricinus communis (castor) seed oil, Copernicia cerifera (Carnauba) wax, Candelilla wax, Theobroma cacao (Cocoa) Seed Butter, isononyl isononanoate, ozokerite, isopropyl titanium triisostearate, polyhydroxystearic acid, iron oxide, titanium dioxide, sodium levulinate, and hydroxypropyl guar.

In some embodiments, the compositions including the non-naturally occurring polypeptides (e.g., recombinant polypeptides and/or purified recombinant polypeptides) can be formulated for topical application. Topical application includes application on skin and/or keratinous tissue. Keratinous tissue includes keratin-containing layers disposed as the outermost protective covering of mammals and includes, but is not limited to, lips, skin, hair, and nails. The topical application can be for cosmetic purpose. The topical formulation can be any type of topical formulation, including, but not limited to, a powder, a cream, a gel, a gel cream, a liquid, a lotion, an oil, and the like. In such embodiments, the composition may further include at least one of a carrier molecule (e.g., vehicle), a preservative, and/or additional ingredients. Any suitable carrier molecules are contemplated, and the exemplary carrier molecule may include water, oil, alcohol, propylene glycol, or emulsifiers. In addition, any suitable preservatives are contemplated, and the exemplary preservatives include zinc oxide, parabens, formaldehyde releasers, isothiazolinones, phenoxyethanol, or organic acids such as benzoic acid, sodium benzoate, or butylene glycol, hexanediol, or potassium sorbate. Such compositions are typically dermatologically acceptable in that they do not have undue toxicity, incompatibility, instability, allergic response, and the like, when applied to skin and/or keratinous tissue Topical skin care compositions of the present disclosure can have a selected viscosity to avoid significant dripping or pooling after application to skin and/or keratinous tissue.

In one aspect, the compositions that comprise non-naturally occurring polypeptides may be personal care products (e.g., a cosmetic). In some embodiments, the compositions are formulated for topical administration. The compositions can contain other cosmetic ingredients suitable for human use. The personal care products may be useful for preventing or treating ultraviolet radiation damage to human skin or hair. The personal care products may be useful for increasing the firmness, elasticity, brightness, hydration, tactile texture, or visual texture of skin and/or stimulate collagen production. The personal care products may be useful for reducing deep lines and wrinkles, reducing fine lines and wrinkles, evening uneven skin tone, increasing skin radiance, reducing photodamage, reducing sagging skin, reducing loss of facial volume, increasing skin barrier function, reducing redness of the skin, reducing skin dryness, reducing peeling or flaking, or increasing expression and/or production of collagen, elastin, fibronectin or laminin. The personal care products may be applied to skin or hair. The compositions include, for example, masks, skin cleaners such as soap, cleansing creams, cleansing lotions, a facial lotion, a body lotion, a shower gel, an antiperspirant, a deodorant, a shave cream, a depilatory, a face oil, a lip oil, a body oil, facial cleansers, cleansing milks, cleansing pads, facial washes, facial and body creams and moisturizers, facial serums, facial and body masks, facial toners and mists, eye creams and eye treatments, an eye serum, exfoliator formulas, lip balms and lipsticks, hair shampoo, hair conditioner and body shampoos, hair and scalp serums, hair mists and sprays, a foundation, a tinted multifunctional cream, eye shadow, concealer, mascara and other color cosmetics.

The compositions that comprise the non-naturally occurring polypeptide can further comprise at least one additional ingredient comprising a topical carrier or a preservative. The topical carrier may comprise a topical carrier selected from the group consisting of liposome, biodegradable microcapsule, lotion, spray, aerosol, dusting powder, biodegradable polymer, mineral oil, triglyceride oil, silicone oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, water, digylcerol (INCI: diglycerin), and fatty acids (e.g., caprylic acid, lauric acid, palmitic acid, stearic acid, oleic acid, linoleic acid, linolenic acid). The preservative may comprise a preservative selected from the group consisting of tocopherol, diiodomethyl-p-tolylsulfone, 2-bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-ethylbicyclooxazolidine, phenoxyethanol, butylene glycol, 1,2 Hexanediol, methyl paraben, sorbic acid, Germaben® II, rosemary extract, EDTA, benzoic acid and salts thereof, and chlorhexidine.

In various aspects, the compositions, formulations, and/or personal care products provided herein are animal-free. For example, the compositions, formulations, and/or personal care products provided herein do not include any ingredients obtained from an animal. In some cases, the compositions, formulations, and/or personal care products provided herein comprise and/or are made from materials obtained from plants or materials with a plant origin. In some cases, the compositions, formulations, and personal care products provided herein comprise materials obtained synthetically or materials with a synthetic origin (e.g., produced in a microbial cell, e.g., a bacterial cell, a yeast cell, a fungal cell). In some cases, the compositions, formulations, and/or personal care products provide herein do not contain Animal Derived Ingredients (ADIs). Thus, the compositions, formulations, and/or personal care products provided herein are free of Bovine Spongiform Encephalopathy (BSE) and/or Transmissible Spongiform Encephalopathies (TSE). In some embodiments, the compositions, formulations, and/or personal care products provided herein are not tested on animals.

In various aspects, the compositions, formulations, and/or personal care products provided herein do not comprise any detectable genetically-modified organisms or any detectable genetically-modified organism genetic material. In some cases, the compositions, formulations, and/or personal care products provided herein are characterized by the absence of live microflora, as determined by a colony forming unit (CFU) assay. In some cases, the compositions, formulations, and/or personal care products provided herein are characterized by the absence of live microflora DNA, as determined by polymerase chain reaction (PCR).

In various aspects, the compositions, formulations, and/or personal care products provided herein do not contain any naturally occurring and/or synthetic chemicals that are known to cause cancer or birth defects or other reproductive harm. A non-limiting list of such ingredients may be found at oehha.ca.gov/proposition-65/proposition-65-list. In various aspects, the compositions, formulations, and/or personal care products provided herein do not contain any carcinogenic, mutagenic, or toxic to reproduction (CMR) substances. In various aspects, the compositions, formulations, and/or personal care products provided herein do not contain a substance of very high concern (SVHC). In various aspects, the compositions, formulations, and/or personal care products do not contain any Convention on International Trade in Endangered Species of Wild Fauna and Flora (CITES) ingredients (e.g., any ingredients derived from, obtained from, or originating from any species protected by CITES). In various aspects, the compositions, formulations, and/or personal care products do not contain any conflict minerals or conflict resources.

In various aspects, the compositions, formulations, and/or personal care products are fragrance-free. In various aspects, the compositions, formulations, and/or personal care products are compliant with the International Fragrance Association (IFRA).

In various aspects, the compositions, formulations, and/or personal care do not contain any known allergens. In some cases, the compositions, formulations, and/or personal care products are free of any source of tree nut or peanut-based materials. In some cases, the compositions, formulations, and/or personal care products are not processed using equipment that has been in contact with tree nut or peanut-based materials. In some cases, the compositions, formulations, and/or personal care products are free of any source of coconut-based materials. In some cases, the compositions, formulations, and/or personal care products are not processed using equipment that has been in contact with coconut-based materials. In some cases, the compositions, formulations, and/or personal care products are free of any source of wheat-based materials. In some cases, the compositions, formulations, and/or personal care products are not processed using equipment that has been in contact with wheat-based materials. In some cases, the compositions, formulations, and/or personal care products are free of any source of gluten (e.g., are gluten-free). In some cases, the compositions, formulations, and/or personal care products are free of any source of lactose or lactose derivatives (e.g., are lactose-free). In some cases, the compositions, formulations, and/or personal care products are free of any source of latex or latex derivatives (e.g., are latex-free).

In various aspects, the compositions, formulations, and/or personal care products are free of one or more ingredient selected from the group consisting of: phthalates, parabens, triclosan, urea, butylated hydroxytoluene (BHT), butylated hydroxyanisole (BHA), formaldehyde, a mixture of methylchloroisothiazolinone and methylisothiazolinone (e.g., Kathon®), mineral oil, phenoxyethanol, petrolatum, monoethanolamine (MEA), diethanolamine (DEA), triethanolamine (TEA), ethylenediaminetetraacetic acid (EDTA), ethylene glycol, sulfates (e.g., sodium lauryl sulfate (SLS), sodium lauryl ether sulfate (SLES)), retinyl palmitate, ethylene oxide, 1,4-dioxane, and any combination thereof. In various aspects, the compositions, formulations, and/or personal care products are free of pesticides. In various aspects, the compositions, formulations, and/or personal care products are free of nanoparticles (“nano-free”). In various aspects, the compositions, formulations, and/or personal care products are free of aflatoxins. In various aspects, the compositions, formulations, and/or personal care products are free of mycotoxins. In various aspects, the compositions, formulations, and/or personal care products are free of poly aromatic hydrocarbons (PAH). In various aspects, the compositions, formulations, and/or personal care products are free of silicones (e.g., cyclosiloxanes). In various aspects, the compositions, formulations, and/or personal care products are not manufactured using any solvents listed in USP <467> or ICH Q3C (R6). In various aspects, the compositions, formulations, and/or personal care products do not contain any volatile organic compounds as defined by the Swiss Ordinance 814.018.

In various aspects, the compositions, formulations, and/or personal care products contain less than 0.5 ppm arsenic. In various aspects, the compositions, formulations, and/or personal care products contain less than 0.1 ppm mercury. In various aspects, the compositions, formulations, and/or personal care products contain less than 0.1 ppm cadmium. In various aspects, the compositions, formulations, and/or personal care products contain less than 2 ppm lead.

In various aspects, the compositions, formulations, and/or personal care products are certified as Vegan. In various aspects, the compositions, formulations, and/or personal care products are certified as Cruelty-Free. In various aspects, the compositions, formulations, and/or personal care products are certified as Halal.

In certain embodiments, provided herein are methods of promoting, maintaining, and/or improving youthful skin (e.g., appearance of skin, texture of skin, etc.) of an individual, comprising applying a composition, a formulation, and/or a personal care product (e.g., containing a non-naturally occurring polypeptide of the disclosure) to the skin of an individual. Promoting and/or maintaining youthful skin may comprise promoting, maintaining, and/or improving the appearance of the skin of an individual. In some cases, the appearance of the skin of the individual, after application of the compositions, formulations, and/or personal care products provided herein (e.g., containing a non-naturally occurring polypeptide of the disclosure) more closely resembles the appearance of the skin of a young individual (e.g., less than 30 years old, less than 25 years old, less than 20 years old, less than 15 years old, etc.). Promoting and/or maintaining youthful skin may comprise promoting, maintaining, and/or improving the texture of the skin of an individual. In some cases, the texture of the skin of the individual, after application of the compositions, formulations, and/or personal care products provided herein (e.g., containing a non-naturally occurring polypeptide of the disclosure) more closely resembles the texture of the skin of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin of an individual to promote, maintain, and/or improve the firmness of the skin. In some cases, the firmness of the skin of the individual, after application of the compositions, formulations, and/or personal care products provided herein (e.g., containing a non-naturally occurring polypeptide of the disclosure) more closely resembles the firmness of the skin of a young individual (e.g., less than 30 years old, less than 25 years old, less than 20 years old, less than 15 years old, etc.). In some cases, promoting, maintaining, and/or improving the firmness of the skin involves increasing the firmness of the skin (e.g., relative to the skin prior to application of the compositions, formulations, and/or personal care products provided herein (e.g., containing a non-naturally occurring polypeptide of the disclosure). In some cases, the firmness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring the resistance of the skin to negative pressure. In some cases, the resistance of the skin to negative pressure is measured by using a Cutometer®.

In various aspects, the methods provided herein comprise applying the compositions, formulations, and/or personal care products (e.g., containing a non-naturally occurring polypeptide of the disclosure) to the skin of an individual to promote, maintain, and/or improve the elasticity of the skin. In some cases, the elasticity of the skin of the individual, after application of the compositions, formulations, and/or personal care products provided herein (e.g., containing a non-naturally occurring polypeptide of the disclosure) more closely resembles the elasticity of the skin of a young individual (e.g., less than 30 years old, less than 25 years old, less than 20 years old, less than 15 years old, etc.). In some cases, promoting, maintaining, and/or improving the elasticity of the skin involves increasing the elasticity of the skin (e.g., relative to the skin prior to application of the compositions, formulations, and/or personal care products provided herein (e.g., containing a non-naturally occurring polypeptide of the disclosure). In some cases, the elasticity of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring the ability of the skin to return to its original position after deformation. In some cases, the ability of the skin to return to its original position after deformation is measured by using a Cutometer®.

In various aspects, the methods provided herein comprise applying the compositions, formulations, and/or personal care products (e.g., containing a non-naturally occurring polypeptide of the disclosure) to the skin of an individual to promote, maintain, and/or improve the brightness of the skin. In some cases, the brightness of the skin of the individual, after application of the compositions, formulations, and/or personal care products provided herein (e.g., containing a non-naturally occurring polypeptide of the disclosure) more closely resembles the brightness of the skin of a young individual (e.g., less than 30 years old, less than 25 years old, less than 20 years old, less than 15 years old, etc.). In some cases, promoting, maintaining, and/or improving the brightness of the skin involves increasing the brightness of the skin (e.g., relative to the skin prior to application of the compositions, formulations, and/or personal care products provided herein (e.g., containing a non-naturally occurring polypeptide of the disclosure). In some cases, the brightness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin of an individual to promote, maintain, and/or improve the hydration of the skin. In some cases, the hydration of the skin of the individual, after application of the compositions, formulations, and/or personal care products provided herein (e.g., containing a non-naturally occurring polypeptide of the disclosure) more closely resembles the hydration of the skin of a young individual (e.g., less than 30 years old, less than 25 years old, less than 20 years old, less than 15 years old, etc.). In some cases, promoting, maintaining, and/or improving the hydration of the skin involves increasing the hydration of the skin (e.g., relative to the skin prior to application of the compositions, formulations, and/or personal care products provided herein (e.g., containing a non-naturally occurring polypeptide of the disclosure). In some cases, the hydration of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring capacitance of the skin. In some cases, capacitance of the skin is measured by a Corneometer®.

In various aspects, the methods provided herein comprise applying the compositions, formulations, and/or personal care products (e.g., containing a non-naturally occurring polypeptide of the disclosure) to the skin of an individual to promote, maintain, and/or improve tactile texture of the skin. In some cases, tactile texture of the skin of the individual, after application of the compositions, formulations, and/or personal care products provided herein (e.g., containing a non-naturally occurring polypeptide of the disclosure) more closely resembles tactile texture of the skin of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin of an individual to promote, maintain, and/or improve visual texture of the skin. In some cases, visual texture of the skin of the individual, after application of the compositions, formulations, and/or personal care products provided herein (e.g., containing a non-naturally occurring polypeptide of the disclosure) more closely resembles visual texture of the skin of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin of an individual to promote, maintain, and/or improve the collagen content of the skin. In some cases, the collagen content of the skin of the individual, after application of the compositions, formulations, and/or personal care products provided herein (e.g., containing a non-naturally occurring polypeptide of the disclosure) more closely resembles the collagen content of the skin of a young individual (e.g., less than 30 years old, less than 25 years old, less than 20 years old, less than 15 years old, etc.). In some cases, promoting, maintaining, and/or improving the collagen content of the skin involves increasing the collagen content of the skin (e.g., relative to the skin prior to application of the compositions, formulations, and/or personal care products provided herein (e.g., containing a non-naturally occurring polypeptide of the disclosure). In some cases, the collagen content of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%.

In various aspects, the methods provided herein comprise applying the compositions, formulations, and/or personal care products (e.g., containing a non-naturally occurring polypeptide of the disclosure) to the skin of an individual to promote, maintain, and/or improve the elastin content of the skin. In some cases, the elastin content of the skin of the individual, after application of the compositions, formulations, and/or personal care products provided herein (e.g., containing a non-naturally occurring polypeptide of the disclosure) more closely resembles the elastin content of the skin of a young individual (e.g., less than 30 years old, less than 25 years old, less than 20 years old, less than 15 years old, etc.). In some cases, promoting, maintaining, and/or improving the elastin content of the skin involves increasing the elastin content of the skin (e.g., relative to the skin prior to application of the compositions, formulations, and/or personal care products provided herein (e.g., containing a non-naturally occurring polypeptide of the disclosure). In some cases, the elastin content of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%.

In various aspects, the methods provided herein comprise applying the compositions, formulations, and/or personal care products (e.g., containing a non-naturally occurring polypeptide of the disclosure) to the skin of an individual to improve the redness of the skin. In some cases, the redness of the skin of the individual, after application of the compositions, formulations, and/or personal care products provided herein (e.g., containing a non-naturally occurring polypeptide of the disclosure) more closely resembles the redness of the skin of a young individual (e.g., less than 30 years old, less than 25 years old, less than 20 years old, less than 15 years old, etc.). In some cases, improving the redness of the skin involves decreasing the redness of the skin (e.g., relative to the skin prior to application of the compositions, formulations, and/or personal care products provided herein (e.g., containing a non-naturally occurring polypeptide of the disclosure). In some cases, the redness of the skin, after application of the compositions, formulations, and/or personal care products provided herein (e.g., containing a non-naturally occurring polypeptide of the disclosure) is decreased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin of an individual to improve fine lines and/or wrinkles of the skin. In some cases, the fine lines and/or wrinkles of the skin of the individual, after application of the compositions, formulations, and/or personal care products provided herein (e.g., containing a non-naturally occurring polypeptide of the disclosure) more closely resembles the fine lines and/or wrinkles skin of a young individual (e.g., less than 30 years old, less than 25 years old, less than 20 years old, less than 15 years old, etc.). In some cases, improving the fine lines and/or wrinkles of the skin involves decreasing fine lines and/or wrinkles (e.g., decreasing the amount, decreasing the size, etc.) of the skin (e.g., relative to the skin prior to application of the compositions, formulations, and/or personal care products provided herein (e.g., containing a non-naturally occurring polypeptide of the disclosure). In some cases, the fine lines and/or wrinkles of the skin, after application of the compositions, formulations, and/or personal care products provided herein (e.g., containing a non-naturally occurring polypeptide of the disclosure) is decreased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin of an individual to promote, maintain, and/or improve epidermal thickness of the skin. In some cases, the epidermal thickness of the skin of the individual, after application of the compositions, formulations, and/or personal care products provided herein (e.g., containing a non-naturally occurring polypeptide of the disclosure) more closely resembles the epidermal thickness of the skin of a young individual (e.g., less than 30 years old, less than 25 years old, less than 20 years old, less than 15 years old, etc.). In some cases, promoting, maintaining, and/or improving the epidermal thickness of the skin involves increasing the epidermal thickness of the skin (e.g., relative to the skin prior to application of the compositions, formulations, and/or personal care products provided herein (e.g., containing a non-naturally occurring polypeptide of the disclosure). In some cases, the epidermal thickness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by reflectance confocal microscopy. In some cases, reflectance confocal microscopy is performed by a Vivascope®.

In various aspects, the compositions, formulations, and/or personal care products provided herein (e.g., containing a non-naturally occurring polypeptide of the disclosure) increase keratinocyte growth (e.g., proliferation) after application to the skin of an individual. In some cases, keratinocyte growth (e.g., proliferation) is increased by at least about 10%, at least about 20%, at least about 30%, at least about 40%, at least about 50%, at least about 60%, at least about 70%, at least about 80%, at least about 90%, at least about 100%, or greater, after the composition, formulation, and/or personal care product (e.g., as disclosed herein; e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte regeneration after application to the skin of an individual. In some cases, keratinocyte regeneration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte regeneration after application to the skin of an individual. In some cases, keratinocyte regeneration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase collagen production by fibroblasts after application to the skin of an individual. In some cases, collagen production by fibroblasts is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast migration after application to the skin of an individual. In some cases, fibroblast migration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast proliferation after application to the skin of an individual. In some cases, fibroblast proliferation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast adhesion after application to the skin of an individual. In some cases, fibroblast adhesion is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte viability after exposure to urban dust (e.g., when the compositions, formulations, and/or personal care products are applied to the skin prior to urban dust exposure). In some cases, keratinocyte viability after exposure to urban dust is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In some embodiments, the compositions, formulations, and/or personal care products disclosed herein (e.g., containing a non-naturally occurring polypeptide of the disclosure) have anti-oxidative capacity. In some embodiments, the anti-oxidative capacity may be measured using an oxygen radical absorbance capacity (ORAC) assay. In some embodiments, the antioxidative capacity may be measured in Trolox equivalent units. In some embodiments, the antioxidative capacity of the composition may be at least about 50 μM, 100 μM, 150 μM, 200 μM, 250 μM, or more than 250 μM Trolox equivalent units.

In various aspects, the compositions, formulations, and/or personal care products provided herein (e.g., containing a non-naturally occurring polypeptide of the disclosure) increase expression of one or more genes (e.g., one or more genes involved in cell proliferation, cell migration, cell adhesion, etc.) (by a cell present in the skin, e.g., keratinocytes, fibroblasts) after application to the skin of an individual. In some cases, expression of one or more genes (e.g., by a cell present in the skin, e.g., fibroblast, keratinocyte) is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In some embodiments, the one or more genes are involved in a signaling pathway (e.g., involved in cell proliferation, cell migration, cell adhesion). In some cases, the one or more genes are involved in a VEGFA/VEGFR2 signaling pathway. In some cases, the one or more genes involved in a VEGFA/VEGFR2 signaling pathway is selected from the group consisting of: MYOC1, FLII, ROCK1, ROCK2, CLTC, LIMK 1, EGR1, and any combination thereof.

In some cases, the one or more genes are involved in a focal adhesion signaling pathway. In some cases, the one or more genes involved in a focal adhesion signaling pathway is selected from the group consisting of: ITGA3, TNC, LAMC1, FLNA, TLN1, ZYX, DIAPH1, and any combination thereof.

In some cases, the one or more genes are involved in an endothelin signaling pathway. In some cases, the one or more genes involved in an endothelin signaling pathway is selected from the group consisting of: TRIOBP, WNK1, MMP2, VCAN, ACTA2, GNA12, EGR1, and any combination thereof.

In some cases, the one or more genes are involved in an EGF/EGFR signaling pathway. In some cases, the one or more genes involved in an EGF/EGFR signaling pathway is selected from the group consisting of: ATXN2, JAK1, RPS6KA2, ROCK1, SHC1, IQGAP1, PLCG1, and any combination thereof.

In some cases, the one or more genes are involved in a transforming growth factor-beta (TGF-beta) signaling pathway. In some cases, the one or more genes involved in a TGF-beta signaling pathway is selected from the group consisting of: SMURF1, SPTBN1, PAK2, ROCK1, SHC1, TGFBR3, TGFBR1, and any combination thereof.

Provided in certain embodiments herein are (e.g., topical) compositions, formulations, and/or personal care products comprising one or more non-naturally occurring polypeptide provided herein (e.g., for cosmetic use). In some embodiments, the compositions, formulations, and/or personal care products provide any suitable amount of polypeptide provided herein, such as in any suitable amount (e.g., an amount suitable to provide a benefit when given or applied to an individual or a cell). In some specific embodiments, the compositions, formulations, and/or personal care products comprise an amount suitable to provide a beneficial effect to the skin of an individual when (e.g., topically) applied to the skin of the individual. In specific embodiments, the compositions, formulations, and/or personal care products comprise about 0.001% to about 30% w/w of a polypeptide (or non-naturally occurring collagen polypeptide) such as provided herein. In more specific embodiments, the compositions, formulations, and/or personal care products comprise about 0.001% to about 20% w/w of a polypeptide (or non-naturally occurring collagen polypeptide) such as provided herein, about 0.001% to about 10% w/w of a polypeptide (or non-naturally occurring collagen polypeptide) such as provided herein, about 0.001% to about 5% w/w of a polypeptide (or non-naturally occurring collagen polypeptide) such as provided herein, about 0.001% to about 4% w/w of a polypeptide (or non-naturally occurring collagen polypeptide) such as provided herein, about 0.001% to about 3% w/w of a polypeptide (or non-naturally occurring collagen polypeptide) such as provided herein, about 0.001% to about 2% w/w of a polypeptide (or non-naturally occurring collagen polypeptide) such as provided herein, about 0.001% to about 1% w/w of a polypeptide (or non-naturally occurring collagen polypeptide) such as provided herein, about 0.001% to about 0.5% w/w of a polypeptide (or non-naturally occurring collagen polypeptide) such as provided herein, and about 0.001% to about 0.2% w/w of a polypeptide (or non-naturally occurring collagen polypeptide) such as provided herein.

In various embodiments, the concentration or amount of a non-naturally occurring polypeptide (e.g., recombinant protein) provided herein is in a composition, formulation, and/or personal care product provided herein in any suitable amount and may, e.g., vary depending on the use or formulation (e.g., gel, capsule, liquid, powder, etc.). Exemplary concentrations of the non-naturally occurring polypeptides (e.g., recombinant proteins) in the compositions, formulations, and/or personal care products can be at least about 0.01%, at least about 0.05%, at least about 0.1%, at least about 0.2%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% (w/v or w/w). Alternatively and/or additionally, the exemplary concentration of the non-naturally occurring polypeptides (e.g., recombinant proteins) in the compositions, formulations, and/or personal care products can be about 0.01%, about 0.05%, about 0.1%, about 0.2%, about 0.5%, about 1%, about 2%, about 3%, at about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98% (w/v or w/w). Alternatively and/or additionally, the exemplary concentration of the non-naturally occurring polypeptides (e.g., recombinant proteins) in the compositions, formulations, and/or personal care products can range from about 0.01% to about 99%, from about 0.05% to about 99%, from about 0.1% to about 99%, from about 0.1% to about 99%, from about 0.5% to about 99%, from about 0.1% to about 10%, from about 1% to about 99%, from about 5% to about 99%, from about 10% to about 99%, from about 15% to about 99%, from about 20% to about 99%, from about 25% to about 99%, from about 30% to about 99%, from about 35% to about 99%, from about 40% to about 99%, from about 45% to about 99%, from about 50% to about 99%, from about 55% to about 99%, from about 60% to about 99%, from about 65% to about 99%, from about 70% to about 99%, from about 75% to about 99%, from about 80% to about 99%, from about 85% to about 99%, from about 90% to about 99%, from about 95% to about 99%, from about 0.1% to about 90%, from about 1% to about 90%, from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, from about 80% to about 90%, from about 85% to about 90%, from about 20% to about 80%, from about 25% to about 80%, from about 30% to about 80%, from about 35% to about 80%, from about 40% to about 80%, from about 45% to about 80%, from about 50% to about 80%, from about 55% to about 80%, from about 60% to about 80%, from about 65% to about 80%, from about 70% to about 80%, from about 75% to about 80%, from about 70% to about 99%, from about 75% to about 99%, from about 80% to about 99%, etc (w/w or w/v). Alternatively and/or additionally, the exemplary concentration of the non-naturally occurring polypeptides (e.g., recombinant proteins) in the compositions, formulations, and/or personal care products can be less than about 95%, about 90%, about 85%, about 80%, about 75%, about 70%, about 65%, about 60%, about 55%, about 50%, about 45%, about 40%, etc (w/w or w/v).

In some embodiments, the schedule of application varies depending on the purpose, gender, age, or health condition of the subject. For example, in some embodiments, the compositions, formulations, and/or personal care products are applied (e.g., topically) once a day, twice a day, three times a day, up to 6 times a day, every 2 days, every 3 days, every 4 days, every 5 days, every 6 days, etc. Alternatively and/or additionally, in some embodiments, the compositions, formulations, and/or personal care products are applied (e.g., topically) a plurality of times in an irregular interval, or increased interval, or decreased interval. In certain embodiments, the compositions, formulations, and/or personal care products are topically applied in a dose and/or schedule sufficient or effective for promoting, maintaining, and/or improving youthful skin (e.g., appearance, texture, etc.) as provided herein.

### EXAMPLES

### Example 1. Generation of Non-Naturally Occurring Polypeptides of the Disclosure

This example shows the generation of a recombinant polypeptide of the disclosure by genetically engineered microorganisms and the purification process of such generated polypeptides.

The polynucleotides of SEQ ID NOs: 1, 3, 5, and 7 were synthesized and at least one of the polynucleotides were inserted into a pET vector. Overlaps between a pET vector and SEQ ID NOs: 1, 3, 5, and 7 were designed to be between 20 and 30 bp long and added using PCR with the enzyme PRIMESTARR GXL polymerase (takarabio.com/products/pcr/gc-rich-pcr/primestar-gxl-dna-polymerase). The opened pET vector and insert DNA (e.g., polynucleotide of SEQ ID NO: 1) were assembled together into the final plasmid using IN-FUSION® Cloning (takarabio.com/products/cloning/in-fusion-cloning). In all cases, the nucleic acid sequences were preceded by a secretion signal sequence disclosed as SEQ ID NOs: 9, 11, 13, 15, 17, 19, 21, or 23. Plasmid sequences were verified through Sanger sequencing.

Cells were transformed with final plasmids and subsequently cultivated in minimal media and frozen in 1.5 aliquots with vegetable glycerin at a ratio of 50:50 of cells to glycerin. One vial of this frozen culture was revived in 50 ml of minimal media overnight at 37° C., 200 rpm. Formulations of the minimal media in this example are shown in Table 2 and Table 3. Cells were then transferred into 300 ml of minimal media and grown for 6-9 hours to reach an optical density (OD) 600 of 5-10.

The fermentations were performed at various temperature ranging from 25° to 28° C. For some fermentations, the temperature of the fermentation was maintained at a constant temperature and immediately upon completion of fermentation the polypeptide was purified. For other fermentations, the temperature of the fermentations was maintained for a desired period of time and when cell densities of OD600 of 10-20 were reached, the temperature was reduced to induce protein production. Typically, the temperature was reduced from 28° C. to 25° C. Induction was carried out by adding IPTG to the media at concentrations ranging from 0.1-0.5 mM. Fermentations were continued for 40-60 hours.

The recombinant polypeptide was purified as follows: The pH of the fermentation broth was decreased to between 3-3.5 using 5-50% sulfuric acid. The cells were then separated using centrifugation or centrifugation followed by microfiltration. Supernatant of the acidified broth was tested on a polyacrylamide gel and found to contain recombinant polypeptide in relatively high abundance compared to starting pellet. To obtain volume and salt reduction, concentration and diafiltration steps were performed ultrafiltration. Final polypeptide slurry was run on an SDS-PAGE gel to confirm presence of the recombinant polypeptide.

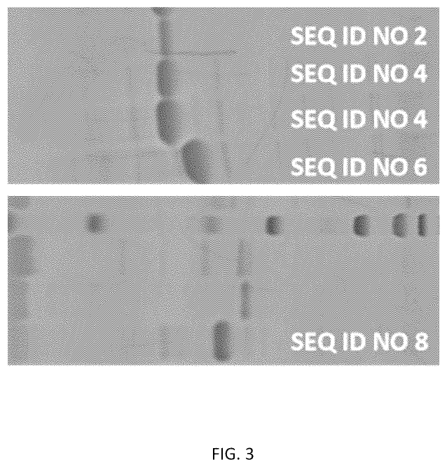

To verify that the desired proteins were produced, supernatants from cultures of microbes carrying SEQ ID NOs: 1, 3, 5, or 7 were collected and purified by decreasing their pH as described above. The acidified broth was analyzed by SDS-PAGE, and bands corresponding to the expected size protein were detected in relative purity. As shown in FIG. 3, a thick and clear band was observed at the expected sizes for each respective protein. Samples were subsequently analyzed for quantifying recombinant polypeptide titers and purity by reverse phase and size exclusion HPLC chromatography and mass spectrometry, which confirmed the correct identity of the respective proteins of interest.

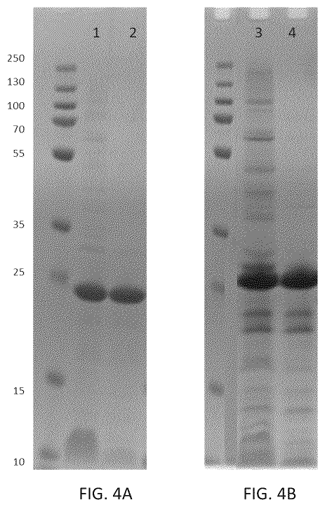

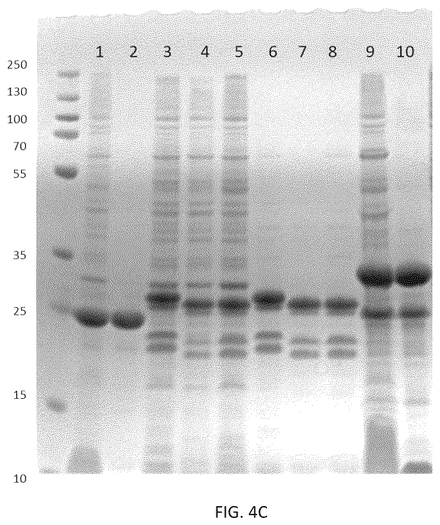

FIGS. 4A-4C depict SDS-PAGE gels of non-naturally occurring polypeptides of the disclosure before and after treatment at pH 3.0. FIG. 4A depicts an SDS-PAGE gel of fermentation supernatant containing a non-naturally occurring polypeptide having an amino acid sequence of SEQ ID NO: 2 before (Lane 1) and after (Lane 2) treatment at pH 3.0. The expected molecular weight of such polypeptide was about 17.9 kDa. The identity of the polypeptide was confirmed by mass spectrometry (data not shown). FIG. 4B depicts an SDS-PAGE gel of fermentation supernatant containing a non-naturally occurring polypeptide having an amino acid sequence of SEQ ID NO: 8 before (Lane 3) and after (Lane 4) treatment at pH 3.0. The expected molecular weight of such polypeptide was about 17.6 kDa. The identity of the polypeptide was confirmed by mass spectrometry (data not shown). FIG. 4C depicts an SDS-PAGE gel of fermentation supernatant containing a non-naturally occurring polypeptide produced in various bacterial host strains having an amino acid sequence of SEQ ID NO: 8 before (Lanes 3-5) and after (Lanes 6-8) treatment at pH 3.0.

### Example 2. Polypeptide Sequence Confirmation of Products and Lack of Hydroxyproline Residues

Mass spectrometry was used to confirm the sequence of a polypeptide of SEQ ID NO: 2 produced by methods according to this disclosure. Table 4 and Table 5 provide the results of peptide mapping of this polypeptide.

TABLE 4
Peptide mapping of the polypeptide of SEQ ID NO: 2.

| | SEQ | | | | | Observed | Mass | Retention |
|---|---|---|---|---|---|---|---|---|
| | ID | | | | Calculated | Mass | Error | Time |
| Label | NO: | Sequence | Range | | Mass (Da) | (Da) | (Da) | (min) |
| T1-Max | 35 | DTGFPGMPGR | 1 | 10 | 1049.4601 | 1049.4598 | −0.000216 | 7.49 |
| T1-2-clipD | 36 | DTGFPGMPGRSGD | 1 | 13 | 1252.5455 | 1292.5471 | 0.001373 | 9.13 |
| T1-2 | 37 | DTGFPGMPGRSGDPGR | 1 | 15 | 1602.7209 | 1602.7206 | 0.00029 | 8.12 |
| T1-3 | 38 | DTGFPGMPGRSGDPGRSGK | 1 | 19 | 1874.8694 | 1874.8634 | −0.005825 | 7.19 |
| T1-4 | 39 | DTGFPGMPGRSGDPGRSGKDGLPGSPGFK | 1 | 29 | 2830.3457 | 2830.3513 | 0.005645 | 8.5 |
| T1-clipT | 40 | TGFPGMPGR | 2 | 10 | 918.43817 | 918.4385 | 0.000225 | 8.38 |
| T1-3-clipG | 41 | GFPGMPGRSGDPGRSGK | 3 | 19 | 1658.7947 | 1658.7977 | 0.003076 | 7.19 |
| T1-3-clipP2 | 42 | PGMPGRSGDPGRSGK | 5 | 19 | 1454.7048 | 1454.7067 | 0.001883 | 7.19 |
| T1-3-clipP1 | 43 | PGRSGDPGRSGK | 3 | 19 | 1169.5901 | 1169.5885 | −0.001693 | 7.19 |
| T2 | 44 | SGDPGR | 11 | 16 | 588.2736 | Not detected | | |
| T2-4 | 45 | SGDPGRSGKDGLPGSPGFK | 11 | 29 | 1814.8911 | 1814.8878 | −0.003236 | 6.62 |
| T3 | | SGK | 17 | 19 | 291.1663 | Not detected | | |
| T4 | 46 | DGLPGSPGFK | 20 | 29 | 974.4941 | Not detected | | |
| T4-5-clipE | 47 | DGLPGSPGFKGE | 20 | 31 | 1159.5509 | 1159.551 | 3.39E−05 | 8.58 |
| T4-5 | 48 | DGLPGSPGFKGEVGQPGSPGLEGHR | 20 | 44 | 2431.188 | 2431.197 | 0.008649 | 8.48 |
| T4-6 | 49 | DGLPGSPGFKGEVGQPGSPGLEGHRGEPGIP | 20 | 59 | 3803.8979 | 3803.9045 | 0.00648 | 9.39 |
| | | GIPGNQGAK | | | | | | |
| T4-7 | 50 | DGLPGSPGFKGEVGQPGSPGLEGHRGEPGIP | 20 | 62 | 4117.0728 | 4117.0747 | 0.002786 | 8.83 |
| | | GIPGNQGAKGQK | | | | | | |
| T4-6-clipG | 51 | DGLPGSPGFKGEVGQPGSPGLEGHRGEPGIP | 21 | 59 | 3688.8711 | 3688.8735 | 0.002824 | 9.01 |
| | | GIPGNQGAK | | | | | | |
| T4-5-clipP | 52 | PGSPGFKGEVGQPGSPGLEGHR | 23 | 44 | 2146.0557 | 2146.0562 | 0.00427 | 8.47 |
| T5 | 53 | GEVGQPGSPGLEGHR | 30 | 44 | 1476.7189 | Not | | |
| | | | | | | detected | | |
| T5-6 | 54 | GEVGQPGSPGLEGHRGEPGIPGIPGNQGAK | 30 | 59 | 2848.4225 | 2848.4215 | −5.51E−05 | 8.28 |
| T5-7 | 55 | GEVGQPGSPGLEGHRGEPGIPGIPGNQGAK | 30 | 62 | 3161.5967 | 3161.5986 | 0.00197 | 7.68 |
| | | GQK | | | | | | |
| T5-6-clipR | 56 | RGEPGIPGIPGNQGAK | 44 | 59 | 1546.8215 | 1546.822 | 0.00052 | 7.68 |
| T6 | 57 | GEPGIPGIPGNQGAK | 45 | 59 | 1391.7277 | Not | | |
| | | | | | | detected | | |
| T6-7 | 58 | GEPGIPGIPGNQGAKGQK | 45 | 62 | 1703.8995 | 1703.8969 | 0.001454 | 7.58 |
| T6-8 | 59 | GEPGIPGIPGNQGAKGQKGEIGPPGLPGAK | 45 | 74 | 2777.4824 | 2777.48 | −0.002657 | 9.08 |
| T6-9 | 60 | GEPGIPGIPGNQGAKGQKGEIGPPGLPGAKG | 45 | 98 | 4955.5239 | 4955.5317 | 0.008078 | 10.72 |
| | | SPGETGLMGPEGSFGLPGAPGPK | | | | | | |
| T6-7-clipP | 61 | PGNQGAKGQK | 53 | 62 | 933.51433 | 933.51324 | −0.001589 | 8.83 |
| T6-8-clipP | 62 | PGNQGAKGQKGEIGPPGLPGAK | 53 | 74 | 2057.1018 | 2057.1055 | 0.003865 | 9.41 |
| T7 | | GQK | 60 | 62 | 332.1928 | Not detected | | |
| T7-8-Matl | 63 | GQKGEIGPPGLPGAK | 60 | 74 | 1418.7882 | 1418.79 | 0.002054 | 8.04 |
| T7-8-clipK | 64 | KGEIGPPGLPGAK | 62 | 74 | 1219.5925 | 1219.691 | −0.001355 | 7.45 |
| T8 | 65 | GEIGPPGLPGAK | 63 | 74 | 1092.6047 | Not detected | | |
| T8-11 | 66 | GEIGPPGLPGAKGSPGETGLMGPEGSFGLPG | 63 | 116 | 4920.4717 | 4920.4771 | 0.005323 | 9.79 |
| | | APGPKGQKGEPGLQGKPGSSGAK | | | | | | |
| T8-clipI | 67 | IGPPGLPGAK | 65 | 74 | 905.53345 | 905.53308 | −0.00042 | 8.71 |
| T9-cation | 68 | GSPGETGLMGPEGSFGLPGAPGPK | 75 | 98 | 2234.0081 | 2233.9998 | −0.008257 | 11.12 |
| T9 | 68 | GSPGETGLMGPEGSFGLPGAPGPK | 75 | 98 | 2197.0593 | Not detected | | |
| T9-10-clipD | 69 | GSPGETGLMGPEGSFGLPGAPGPKGD | 75 | 100 | 2358.1005 | 2353.1003 | −0.000254 | 11.24 |
| T9-10 | 70 | GSPGETGLMGPEGSFGLPGAPGPKGDK | 75 | 101 | 2496.1356 | 2496.1387 | −0.006757 | 10.11 |
| T9-clipT | 71 | TGLMGPEGSFGLPGAPGPK | 80 | 98 | 1768.8818 | 1768.88 | −0.001801 | 11.1 |
| T9-clipG | 72 | GLMGPEGSFGLPGAPGPK | 81 | 98 | 1667.8341 | 1667.3339 | −0.00031 | 11.1 |
| T9-clipS | 73 | SFGLPGAPGPK | 88 | 98 | 1026.5498 | 1026.5435 | −0.00134 | 8.97 |
| T10-11 | 74 | GDKGEPGLQGKPGSSGAK | 99 | 115 | 1668.8431 | 1668.8383 | −0.004932 | 5.46 |
| T11-11- | 75 | KGEPGLQGKPGSSGAK | 101 | 115 | 1498.7947 | 1496.7939 | −0.000623 | 5.33 |

| | | | | | | |
|---|---|---|---|---|---|---|
| T11 | 76 | GEPGLQGKPGSSGAK | 102 | 115 | 1369.707 | Not detected |

TABLE 5
Peptide mapping of the polypeptide of SEQ ID NO: 2.

| | SEQ | | | | | Observed | Mass | Retention | |
|---|---|---|---|---|---|---|---|---|---|
| | ID | | | | Calculated | Mass | Error | Time | Intensity |
| Label | NO: | Sequence | Range | | Mass (Da) | (Da) | (Da) | (min) | {counts} |
| T11-13 | 77 | GEPGLQGKPGSSGAKSEPGGPGAP | 102 | 143 | 3839.908 | 3839.9138 | 0.007516 | 7.99 | 15,528.34 |
| | | GEPGYPGIPGTQGIKGDK | | | | | | | |
| T12 | 78 | GEPGGPGAPGEPGYPGIPGTGGIK | 117 | 140 | 2189.0752 | 2189.0723 | −0.003083 | 9.65 | 145,576.30 |
| T12-13 | 79 | GEPGGPGAPGEPGYPGIPGTGGIKG | 117 | 143 | 2489.2188 | 2489.219 | 0.000101 | 8.74 | 45,482.75 |
| | | DK | | | | | | | |
| T12-14- | 80 | PGEPGYPGIPGTQGIKGDKGSQGE | 125 | 154 | 2923.4424 | 2923.4473 | 0.005102 | 7.56 | 18,260.02 |
| clipP4 | | SGIQGR | | | | | | | |
| T12-14- | 81 | PGYPGIPGTQGIKGDKGSQGESGI | 128 | 154 | 2640.3257 | 2540.3262 | 0.000495 | 7.58 | 18,118.65 |
| clipP3 | | QGR | | | | | | | |
| T12-14- | 82 | PGIPGTQGIKGDKGSQGESGIQGR | 131 | 154 | 2323.188 | 2323.1851 | −0.0029 | 8.33 | 18,247.45 |
| clipP2 | | | | | | | | | |
| T12-14- | 83 | PGTQGIKGDKGSQGESGIQGR | 134 | 154 | 2056.0298 | 2056.0305 | 0.000825 | 7.56 | 24,994.64 |
| clipP1 | | | | | | | | | |
| T12-15- | 84 | PGTQGIKGDKGSQGESGIQGRK | 134 | 155 | 2056.0298 | 2056.0308 | 0.00072 | 8.33 | 13,544.56 |
| clipP | | | | | | | | | |
| T13 | | GDK | 141 | 143 | 319.1512 | Not detected | | | |
| T13-14 | 85 | GDKGSQGESGIQGR | 141 | 154 | 1374.6488 | 1374.6508 | 0.001875 | 5.23 | 66,051.58 |
| T13-15 | 86 | GDKGSQGESGIQGRK | 141 | 155 | 1374.5488 | 1374.6508 | 0.001873 | 5.23 | 66,051.58 |
| T13-16 | 87 | GDKGSQGESGIQGRKGEK | 141 | 158 | 1816.9027 | 1816.899 | −0.00382 | 2.88 | 2,494.48 |
| T13-17 | 88 | GDKGSQGESGIQGRKGEKGR | 141 | 160 | 2030.0253 | 2030.022 | −0.003288 | 2.72 | 2,147.75 |
| T13-14- | 89 | KGSQGESGIQGR | 143 | 154 | 1202.6003 | 1202.5985 | −0.00199 | 3.44 | 2,745.49 |
| clipK | | | | | | | | | |
| T14 | 90 | GSQGESGIQGR | 144 | 154 | 1075.5126 | Not detected | | | |
| T14-15 | 91 | GSQGESGIQGRK | 144 | 155 | 1202.6003 | 1202.5983 | −0.00199 | 3.44 | 2,745.49 |
| T14-16 | 92 | GSQGESGIQGRKGEK | 144 | 158 | 1516.7594 | 1516.7587 | −0.000831 | 3.02 | 1,968.24 |
| T14-17 | 93 | GSQGESGIQGRKGEKGR | 144 | 160 | 1729.882 | 1729.8775 | −0.00443 | 2.79 | 2,134.92 |
| T15 | | K | 155 | 155 | 147.1128 | Not detected | | | |
| T15-18 | 94 | KGEKGRQGNPGLQGTEGLR | 155 | 173 | 1981.0453 | 1981.0386 | −0.006898 | 5.56 | 19,503.56 |
| T15-19 | 95 | KGEKGRQGNPGLQGTEGLRGEQGEK | 155 | 179 | 2609.3269 | 2609.3267 | −0.000342 | 5.51 | 112,503.40 |
| T16-18 | 96 | GEKGRQGNPGLQGTEGLR | 156 | 173 | 1852.9503 | 1852.9458 | −0.004281 | 5.76 | 1,739.51 |
| T16-20 | 97 | GEKGRQGNPGLQGTEGLRGEQGEKG | 156 | 182 | 2795.3911 | 2795.5877 | −0.0033 | 5.51 | 8,967.19 |
| T17 | | EKGR | 159 | 160 | 232.1404 | Not detected | | | |
| T17-18- | 98 | GRQGNPGLQGTEGL | 159 | 172 | 1382.6902 | 1582.6887 | −0.001582 | 7.58 | 12,756.85 |
| clipL | | | | | | | | | |
| T17-18 | 99 | GRQGNPGLQGTEGLR | 159 | 173 | 1558.7914 | 1538.7919 | 0.000687 | 6.51 | 25,248.48 |
| T17-19 | 100 | GRQGNPGLQGTEGLRGEQGEK | 159 | 179 | 2167.073 | 2167.0701 | −0.002904 | 5.83 | 106,768.80 |
| T17-20 | 101 | GRQGNPGLQGTEGLRGEQGEKGEK | 159 | 182 | 2481.2319 | 2481.2329 | 0.000921 | 5.59 | 38,064.79 |
| T17-21- | 102 | GRQGNPGLQGTEGLRGEQGEKGEKGD | 159 | 184 | 2653.2805 | 2653.2791 | −0.001494 | 5.33 | 10,397.58 |
| clipD | | | | | | | | | |
| T18 | 103 | QGNPGLQGTEGLR | 161 | 173 | 1526.575 | Not detected | | | |
| T18-19 | 104 | QGNPGLQGTEGLRGEQGEK | 161 | 179 | 1936.9238 | 1936.9227 | −0.001056 | 7.68 | 14,110.98 |
| T18-20 | 105 | QGNPGLQGTEGLRGEQGEKGEK | 161 | 182 | 2268.1094 | 2268.1101 | 0.000698 | 5.98 | 60,817.64 |
| T18-21- | 106 | QGNPGLQGTEGLRGEQGEKGEKGD | 161 | 184 | 2440.158 | 2440.1602 | 0.002185 | 6.14 | 17,499.44 |
| clipD | | | | | | | | | |
| T18-21- | 107 | RGEQGEKGEKGDPGIR | 173 | 188 | 1711.8601 | 1711.8606 | 9.05E−05 | 5.06 | 7,889.07 |
| clipR | | | | | | | | | |
| T19 | 108 | GEDGEK | 174 | 179 | 647.2995 | Not detected | | | |
| T19-23 | 109 | GEQGEKGEKGDPGIR | 174 | 188 | 1555.759 | 1555.7548 | −0.00416 | 5.3 | 24,352.07 |
| T20 | | GEK | 180 | 182 | 333.1768 | Not detected | | | |
| T20-21 | 110 | GEKGDPGIR | 180 | 188 | 927.47742 | 927.47589 | −0.001604 | 5.25 | 156,253.20 |

| | | | | | | |
|---|---|---|---|---|---|---|
| T21 | 111 | GDPGIR | 183 | 188 | 614.3256 | Not detected |

Analysis was also performed to evaluate any amino acid or peptide modifications present in the produced polypeptide of SEQ ID NO: 2 (Table 6). In a few instances, additional confirmatory analyses were performed to differential methionine oxidation from the presence of hydroxyproline residues. For example, based upon the fragmentation results from MS/MS scans, the tryptic peptide T1 (sequence DTGFPGMPGR (SEQ ID NO: 35)) was shown to contain a methionine oxidation rather than a proline hydroxylation. Based on such results, it was conclusively determined that tryptic peptide 1 (T1) has oxidation at methionine position 7 and no evidence of hydroxyproline at position 5 or 8. Similarly, where there is another methionine in position 83 in tryptic peptide 9 (T9), there were no detectable levels of methionine oxidation, hydroxyproline in positions 77, 85, 92, 95, and 97, or hydroxylysine at position 98 of the polypeptide. Accordingly, the truncated collagen polypeptides of the present disclosure also differ from naturally occurring collagen polypeptides in their lack of hydroxyproline residues.

TABLE 6
Analysis of amino acid and peptide modifications of the polypeptide of SEQ ID NO: 2.

| | | | | Relative |
|---|---|---|---|---|
| | | | Intensity | Instensity |
| Modifications | Label | Sequence | (counts) | (%) |
| Oxidation Met, Missed Cleavages, | T1 | Oxidation (M) | 84,835.78 | 3.29 |
| N and C terminal Clips | | Clippped (T) | 4,768.16 | 0.18 |
| | T1-2 | Missed Cleavage, Clippped (D) | 126,314.00 | 4.90 |
| | | Missed Cleavage | 577,210.30 | 22.38 |
| | T1-3 | 2 Missed Cleavages | 1,090,023.00 | 42.26 |
| | | 2 Missed Cleavages, Clipped (G) | 228,217.00 | 8.85 |
| | | 2 Missed Cleavages, Clipped (P1) | 436,082.90 | 16.91 |
| | | 2 Missed Cleavages, Clipped (P2) | 25,095.66 | 0.97 |
| | T1-4 | 3 Missed Cleavages | 6,936.58 | 0.27 |
| Missed Cleavages, N and | T4 | Unmodified | 233,635.20 | 23.83 |
| C Terminal Clips | T4-5 | Missed Cleavgae, Clipped (E) | 3,738.19 | 0.38 |
| | | Missed Cleavage | 112,201.00 | 11.45 |
| | T4-6 | 2 Missed Cleavages | 441,091.10 | 44.99 |
| | | 2 Missed Cleavages, Clipped (P) | 14,717.96 | 1.50 |
| | T4-7 | 3 Missed Cleavages | 114,096.20 | 11.64 |
| | | 3 Missed Cleavages, Clipped (G) | 60,853.30 | 6.21 |
| Missed Cleavages, N | T5 | Unmodified | 4,628.28 | 1.63 |
| Terminal Clip | T5-6 | Missed Cleavage | 71,435.91 | 25.11 |
| | | Missed Cleavage, Clipped (R) | 3,424.51 | 1.20 |
| | T6 | Unmodified | 177,768.80 | 62.48 |
| | T5-7 | 2 Missed Cleavages | 14,067.67 | 4.94 |
| | T6-7 | Missed Cleavage | 13,208.12 | 4.64 |
| Missed Clevages, N Terminal Clips, | T6-8 | 2 Missed Cleavages | 24,234.60 | 26.78 |
| Methyl Ile, Dehydrated Gln | T6-9 | 3 Missed Cleavages | 9,805.94 | 10.83 |
| | T7-8 | Missed Cleavage, Clipped (P) | 4,871.83 | 5.38 |
| | | Missed Cleavage, Methyl (I) | 3,103.51 | 3.43 |
| | | Missed Cleavage, Clipped (K) | 11,881.92 | 13.13 |
| | T7-9 | 2 Missed Cleavages, Clipped (P) | 1,757.15 | 1.94 |
| | T8 | Dehydrated (E) | 6,818.71 | 7.53 |
| | | Clipped (I) | 28,037.06 | 30.98 |
| Cation K, Missed Cleavgaes, | T9 | Cation K | 48,341.35 | 19.07 |
| N Terminal Clips | | Clipped (T) | 5,679.79 | 2.24 |
| | | Clipped (G) | 1,873.69 | 0.74 |
| | | Clipped (S) | 904.06 | 0.36 |
| | T9-10 | Missed Cleavage | 99,126.63 | 39.11 |
| | | Missed Cleavage, Clipped (D) | 10,231.43 | 4.04 |
| | T10-11 | Missed Cleavage, Clipped (K) | 5,921.06 | 2.34 |
| | T8-11 | 3 Missed Cleavages | 81,370.18 | 32.11 |
| Missed Cleavages | T11 | Unmodified | 40,875.16 | 72.47 |
| | T11-13 | 2 Missed Cleavages | 15,528.34 | 27.53 |
| Missed Cleavages, | T12 | Unmodified | 145,576.30 | 51.93 |
| N Terminal Clips | T12-13 | Missed Cleavage | 45,482.75 | 16.23 |
| | T12-14 | 2 Missed Cleavages, Clipped (P4) | 18,260.02 | 6.51 |
| | | 2 Missed Cleavages, Clipped (P3) | 16,118.65 | 5.75 |
| | | 2 Missed Cleavages, Clipped (P2) | 16,247.45 | 5.80 |
| | | 2 Missed Cleavages, | 24,994.64 | 8.92 |
| | T12-15 | 3 Missed Cleavages, Clipped (P) | 13,644.56 | 4.87 |
| Missed Cleavages | T13-14 | Missed Cleavage | 66,051.58 | 36.91 |
| | T13-16 | 3 Missed Cleavages | 2,494.46 | 1.39 |
| | T13-17 | 4 Missed Cleavages | 2,147.75 | 1.20 |
| | T14 | Unmodified | 101,400.00 | 56.67 |
| | T14-15 | Missed Cleavages | 2,745.49 | 1.53 |
| | T14-17 | 3 Missed Cleavages | 2,134.92 | 1.19 |
| | T14-16 | 2 Missed Cleavages | 1,968.24 | 1.10 |

### Example 3. Preparation of a Powder Comprising a Non-Naturally Occurring Polypeptide of the Disclosure for Use in Beauty and Personal Care Products

This example demonstrates a preparation of a powder comprising a non-naturally occurring polypeptide having an amino acid sequence according to SEQ ID NO: 8. Following fermentation that achieved a suitable cell density and protein expression level (see, e.g., Example 1), the fermentation broth was chilled until ≤15° C. was reached. At this point, a concentrated sulfuric acid solution (98.5% wt) was titrated into the fermentor to reduce the broth pH to 3.0-3.2. Centrifugation was used as the primary broth clarification step to remove the cell biomass and larger cell debris. Microfiltration was then used to remove any residual cells and cell debris in the product-containing stream from centrifugation. Ultrafiltration was used to remove residual salts, sugars, soluble fermentation byproducts, and water from the microfiltration permeate stream. Additional steps included treatment with activated carbon to de-color and de-odorize the protein concentrate, preservative addition, pH adjustment (to pH 4-5) and sterile filtration. The formulated protein concentrate then was spray dried to remove most of the water and to generate the final collagen powder. The resulting collagen powder was then qualified according to the specifications in Table 7.

TABLE 7
Specifications of collagen powder.

| Parameter | Method | Specification |
|---|---|---|
| Appearance | Visual | White to pale yellow powder |
| pH (1% solution) | AOAC OMA | 4.0-5.0 |
| | 943.02/981.12 | |
| Collagen Content | AOAC OMA | ≥70% |
| | 992.15 | |
| (weight %) | (N × 5.55) | |
| Moisture Content | AOAC OMA | <10% |
| (weight %) | 925.09 | |
| Aerobic Plate Count | AOAC OMA | <500 CFU/g |
| | 990.12 | |
| Yeast & Mold | AOAC OMA | <100 CFU/g |
| | 2014.05 | |
| Bacillus cereus | FDA BAM | <10 CFU/g |
| Escherichia coli | USP <62> | Absent |
| Pseudomonas aeruginosa | USP <62> | Absent |
| Salmonella | USP <62> | Absent |
| Staphylococcus aureus | USP <62> | Absent |

### Example 4. Preparation of a Collagen Solution for Use in Beauty and Personal Care Products

The collagen powder from Example 3 was then used to prepare a variety of formulations suitable for incorporation into beauty and personal care products. The standard solution was a 2% solution of the collagen. Exemplary formulations were made with butylene glycol formulations (Table 8) and glycerol formulations (Table 9) as follows. These 2% solutions were than readily analyzed and suitable for further formulation of beauty and personal care products.

TABLE 8
Butylene Glycol Formulation.

| | | Current | |
|---|---|---|---|
| Ingredient | CAS # | concentration | Range |
| Water/Aqua | 7732-18-5 | 90% | 78-90% |
| Butylene glycol | 107-88-0 | 5% | 0-10% |
| 1,2-Hexanediol | 6920-22-5 | 3% | 0-10% |
| Collagen | | 2% | 2% |

TABLE 9
Glycerol Formulation.

| | | Current | |
|---|---|---|---|
| Ingredient | CAS # | concentration | Range |
| Water/Aqua | 7732-18-5 | 78% | 17-78% |
| Glycerol | 56-81-5 | 20% | 20-80% |
| Sodium benzoate | 532-32-1 | 0.5% | 0-1% |
| Collagen | | 2% | 2% |

The resulting 2% collagen solution was then qualified according to the specifications described in Table 10.

TABLE 10
Specifications of a 2% collagen solution.

| Parameter | Method | Specification |
|---|---|---|
| Appearance | Visual | Colorless to |
| | | pale yellow, |
| | | clear solution |
| pH | AOAC OMA | 4.0-5.0 |
| | 943.02/981.12 | |
| Collagen content in | AOAC OMA | ≥70% |
| Collagen powder | 992.15 | |
| | (N × 5.55) | |
| Sturgeon Collagen | Weight % | 1.8%-2.2% |
| concentration | calculation | |
| in solution | | |
| Color | Spectrophotometer | Gardner value < 3 |
| Aerobic Plate Count | AOAC OMA | <100 CFU/g |
| | 990.12 | |
| Yeast & Mold | AOAC OMA | <100 CFU/g |
| | 2014.05 | |
| Bacillus cereus | FDA BAM | <10 CFU/g |
| Escherichia coli | USP <62> | Absent |
| Pseudomonas aeruginosa | USP <62> | Absent |
| Salmonella | USP <62> | Absent |
| Staphylococcus aureus | USP <62> | Absent |

### Example 5. Preparation of Various Cosmetic Products from a Sturgeon Collagen Powder

Powders comprising non-naturally occurring polypeptides of the present disclosure can be used to prepare a variety of cosmetic products for beauty and personal care as described herein. This example demonstrates generally applicable formulations with the use of the non-naturally occurring polypeptides of the present disclosure.

### Preparation of a Formulation with a Powder Comprising a Non-Naturally Occurring Polypeptide of the Disclosure

### Mixing and Hydration

Each formulation was started by mixing ingredients prior to adding the powder. With mixing in progress, the powder was then gradually added. Faster hydration of the powder was achieved with the use of a disperser disk or a high-shear mixer. Alternatively, the powder was hydrated in a concentrated premix prior to its incorporation in the formulation. To facilitate hydration and prevent the formation of lumps, the powder can be premixed into a slurry with a liquid ingredient (e.g., glycerin, propanediol) before hydration.

### Temperature and pH

The powder was incorporated after any neutralization steps of acidic or alkaline components. When exposing the polypeptides to higher temperatures (up to 80° C.) during the formulation process, the pH of the formula was verified to be equal to or higher than 5.0 for optimal results.

The powder can also be dispersed in anhydrous systems using a high-shear mixer. The formulation is mixed prior to adding the powder. With mixing in progress, the powder is gradually added until homogenously dispersed. If finer particles are required, a milling step may need to be applied.

### Preparation of a Formulation with a 2% Solution of a Non-Naturally Occurring Polypeptide of the Disclosure

### Mixing and Hydration

A 2% solution of a non-naturally occurring polypeptide having an amino acid sequence of SEQ ID NO: 8 was also readily incorporated into the aqueous phase of a formulation with standard processing tools.

### Temperature and pH

Best results were achieved when the solutions were incorporated into the formulation below 40° C. and after any neutralization steps of acidic or alkaline components.

TABLE 11
Gel cream formulation containing 0.1% (w/w) of a non-
naturally occurring polypeptide having an amino acid
sequence of SEQ ID NO: 8 as active ingredient.

| Phase | Ingredient | Amount (% w/w) |
|---|---|---|
| Water phase | | |
| | Water | 77.65 |
| | Levulinic Acid, Water, | 4.00 |
| | Glycerin, Sodium | |
| | Levulinate blend | |
| | Polyglyceryl-3 Methylglucose | 2.00 |
| | Distearate | |
| | Glyceryl Undecylenate | 2.00 |
| Oil phase | | |
| | Simmondsia Chinensis | 2.00 |
| | (Jojoba) Seed Oil | |
| | Polyacrylate Crosspolymer | 1.25 |
| | Squalane | 1.00 |
| Active ingredient | | |
| phase | | |
| | Water | 10.00 |
| | Powder of a polypeptide | 0.10 |
| | of SEQ ID NO: 8. | |

TABLE 12
Aqueous gel formulation containing 0.1% (w/w) of a
non-naturally occurring polypeptide having an amino
acid sequence of SEQ ID NO: 8 as active ingredient.

| Ingredient | % wt. | Phase |
|---|---|---|
| Water | 45.0 | A |
| Sodium Hyaluronate | 0.1 | |
| Carbomer (Carbopol ® Ultrez 30 Polymer) | 0.4 | B |
| Glycerin | 1.0 | C |
| Phenoxyethanol | 1.0 | |
| Pentylene Glycol (Hydrolite ® 5 Green) | 0.5 | |
| Water | 45.0 | D |
| Polypeptide having amino acid sequence | 0.1 | |
| of SEQ ID NO: 8 (powder) | | |
| Water (and) Sodium | 0.5 | E |
| Lauryl Sulfoacetate (and) Pentylene Glycol (and) | | |
| Sodium Oleoyl Sarcosinate (and) Sodium Chloride (and) | | |
| Sodium Oleate (SymSol ® PF-3) | | |
| Water (And) Sodium Hydroxide (10N) | PH | F |
| Water | QS | G |
| | 100 | |

TABLE 13
Lipstick formulation containing 0.1% (w/w) of a non-
naturally occurring polypeptide having an amino acid
sequence of SEQ ID NO: 8 as active ingredient.

| Ingredient | % wt. | Phase |
|---|---|---|
| Ricinus Communis (Castor) Seed Oil | 25 | A |
| Copernicia Cerifera (Carnauba) Wax | 5 | |
| Candelilla Wax | 7 | |
| Theobroma cacao (Cocoa) Seed Butter | 50.9 | B |
| Red 7 Lake (And) Isononyl Isononanoate (And) Ozokerite | 5 | C |
| (And) Isopropyl | | |
| Titanium Triisostearate (And) Polyhydroxystearic Acid | | |
| INWP45R7C | | |
| Iron Oxides (CI 77491) (And) Isononyl Isononanoate (And) | 3 | |
| Ozokerite (And) Isopropyl | | |
| Titanium Triisostearate (And) Polyhydroxystearic Acid | | |
| INWP75ER | | |
| Titanium Dioxide (And) Isononyl Isononanoate (And) | 4 | |
| Ozokerite (And) Isopropyl | | |
| Titanium Triisostearate (And) Polyhydroxystearic Acid | | |
| INWP65U | | |
| Polypeptide having an amino acid sequence of SEQ ID | 0.1 | D |
| No: 8 (powder) | | |
| | 100 | |

### Example 6. Determination of Processing Parameters and Suitability for Use of a Powder of a Non-Naturally Occurring Polypeptide of the Disclosure in Various Cosmetic Products

### Solubility

The solubility of a powder comprising a non-naturally occurring polypeptide having an amino acid sequence of SEQ ID NO: 8 in different solvents was tested at a concentration of 0.1% (w/w).

Table 14 below summarizes the solubility results.

TABLE 14
Solubility results

| Solvent | Level of Solubility |
|---|---|
| Water | Soluble |
| Glycerin | Soluble |
| 1,2-propanediol | Soluble |
| 1,2-pentanediol | Limited |
| 1,5-pentanediol | Limited |
| Denatured alcohol | Limited |
| Octyldodecanol | Limited |
| Ricinus Communis (Castor) Seed Oil | Limited |
| Helianthus Annuus (Sunflower) Seed Oil | Limited |
| C12-15 alkyl benzoate | Limited |

The levels of solubility were defined as follows:

Soluble: a clear mixture was obtained after the incorporation of the polypeptide and homogenization.

Limited solubility: significant quantity of suspended particles or high level of haze was observed after the incorporation of the polypeptide and homogenization.

### Dissolution Time Study; Effect of Processing Parameters in Solubility, Mixing, and Hydration of Powder

Deionized water was adjusted to different pH levels using citric acid or sodium hydroxide. The water at different pH levels was heated to different temperatures. The protein was incorporated into the water at a specific pH and temperature, mixed by an overhead mixer (RW20, IKA) at a predefined speed (207±3 rpm), and the dissolution time was recorded with a timer at the time no visible solid material was observed. Color was measured using a spectrophotometer (CM-5, Konica Minolta) and reported in a Gardner scale. Turbidity was measured with a turbidity meter (2100Q, Hach) and reported in NTU. Viscosity was measured using a rheometer (Discovery HR-2, TA instruments), in peak hold mode (60 s) at a shear rate of 10 s⁻¹ with parallel plates at 25° C.

### Variables Tested:

Polypeptide concentration: 0.1% to 1% (w/w)
Initial pH: 3 to 10
Temperature of incorporation: 25° C. to 80° C.
Mixing speed of 207+/−3 rpm

### Outputs:

Dissolution time, final pH, turbidity, color. Such studies can be used to determine optimal parameters for formulation of cosmetic products. It was determined overall that increasing temperature reduces dissolution times, but it can increase turbidity at lower pH. Increasing polypeptide content was found to increase dissolution time, turbidity, and color impact. Increasing initial pH was found to reduce turbidity.

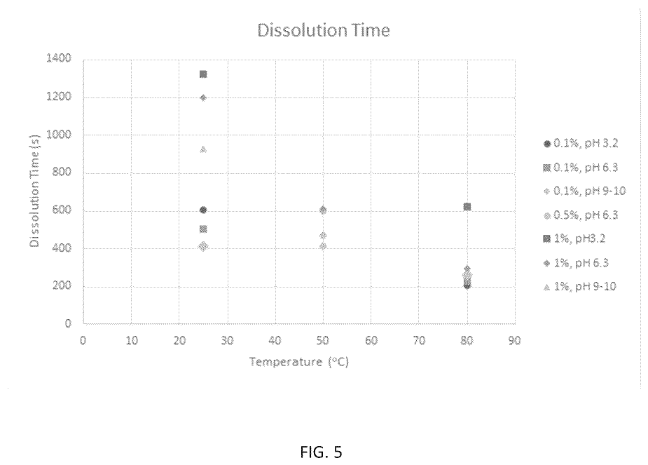

Dissolution time: Increasing the polypeptide concentration was found to increase the dissolution time, and increasing the temperature was found to reduce the dissolution time, as shown in FIG. 5.

Final pH: The polypeptide was found to have a buffering effect that impacts the final pH.

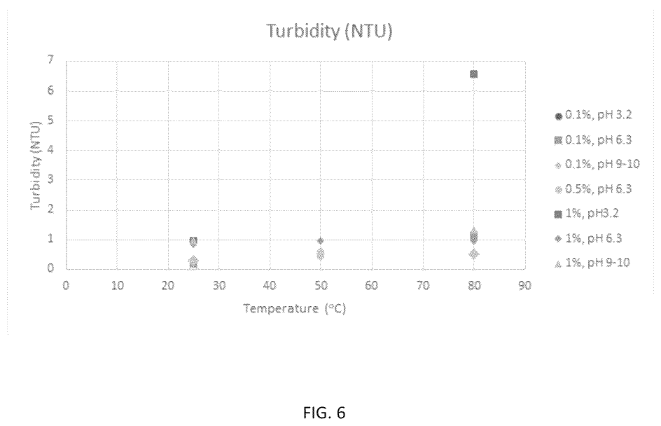

Turbidity: It was determined that increasing temperature and reducing pH can lead to precipitation and increased turbidity. Polypeptide concentration also has a positive impact on turbidity, as shown in FIG. 6.

Color: The primary driver of color variation was found to be polypeptide concentration.

### Effect of pH and Temperature in Formulating Powder/Stress Testing

10 mg/ml concentrated polypeptide solution (containing a polypeptide having an amino acid sequence of SEQ ID NO: 8) was treated at a range of PH levels and temperatures for 4 hours to determine the effect on the quantity (mg/ml) and % full length polypeptide as measures for degradation under those conditions. No difference in color was observed generally. Insoluble particles were observed for 80° C. and pH less than 4.0-treated samples.

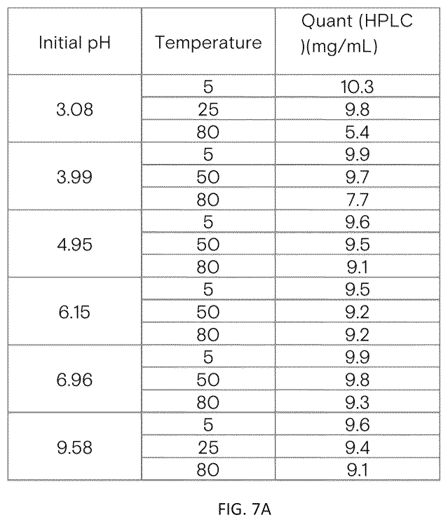

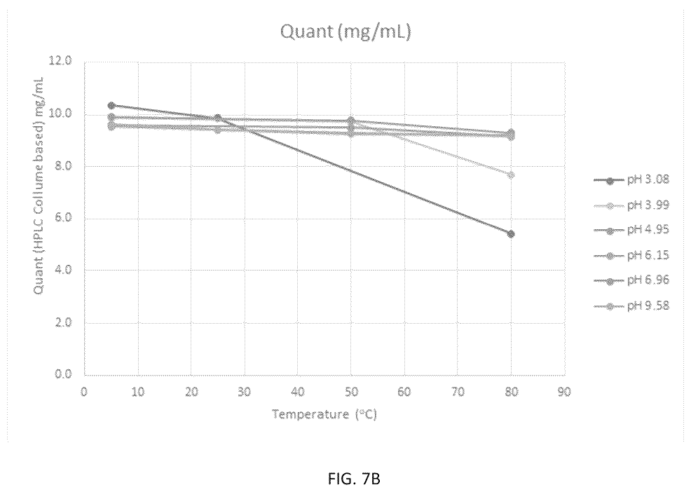

FIG. 7A and FIG. 7B show the stability with increased temperatures at various pH levels.

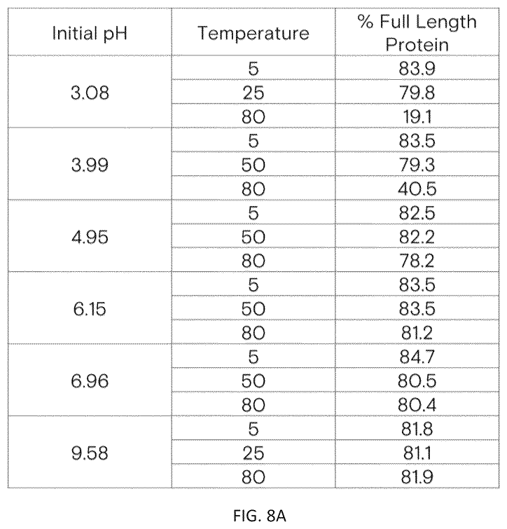

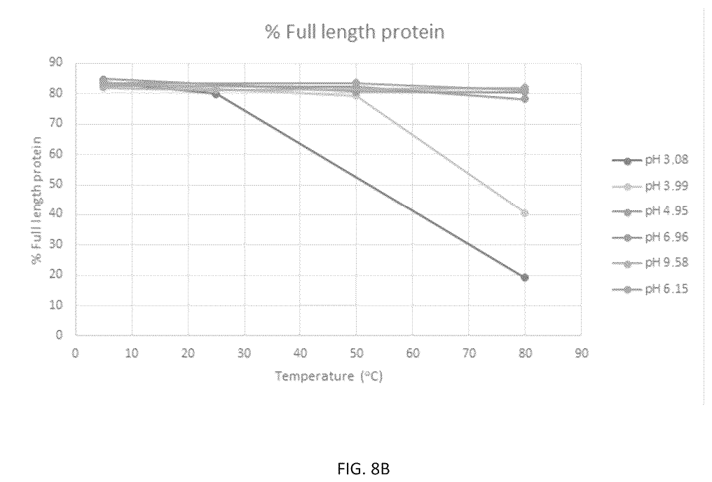

FIG. 8A and FIG. 8B show the stability of full length polypeptide with increased temperatures at various pH levels.

Combined studies showed that the polypeptide was preferably added at the end of the formulation process, prior to emulsification, or in a phase that is heated up to 80° C. if pH is greater than or equal to 5.0. If pH is less than 4.0, the polypeptide was preferably incorporated at the end of the formulation process; if pH was 4.0-5.0, the polypeptide was preferably added below 50° C., and if pH was greater than or equal to 5.0, the polypeptide may be heated during the process up to 80° C.

The impact of pH or pH-induced protein degradation on viscosity was evaluated with solution containing 1% (w/w) of the polypeptide.

Viscosity profile of the product was measured using a rheometer (Discovery HR-2, TA Instruments), using parallel plates with a 1 mm gap. In flow sweep mode, the shear rate varied from 0.1 s⁻¹ to 1000 s⁻¹, and the viscosity was expressed as a function of the shear rate. All samples were tested at 25° C.

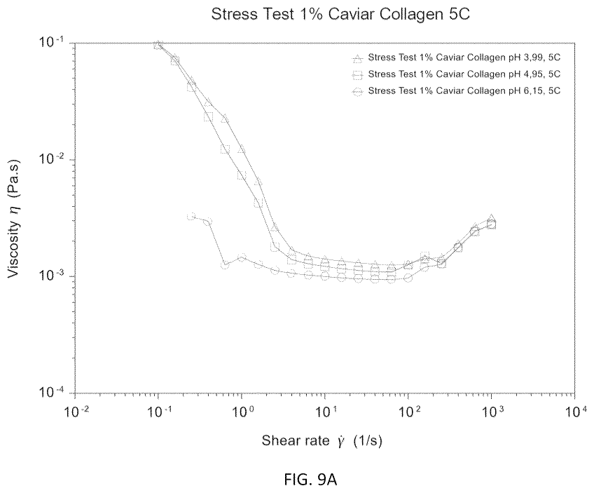

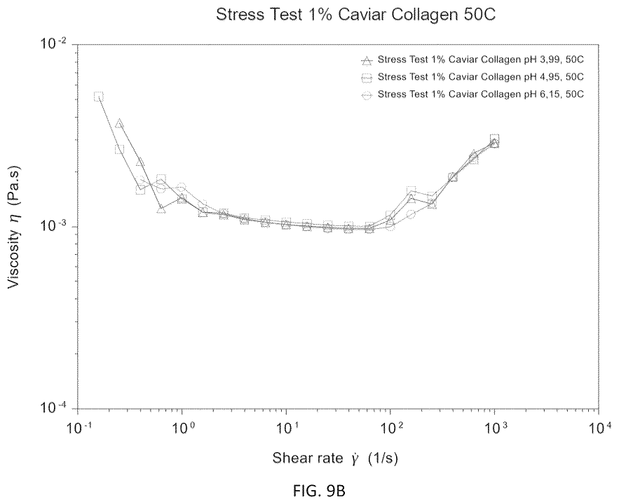

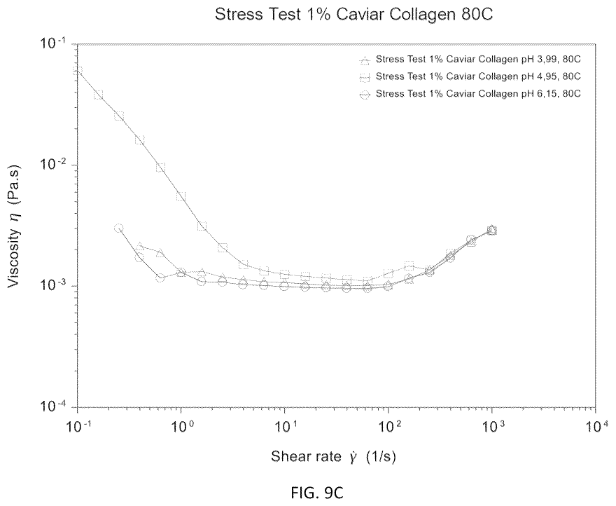

Effect of pH on viscosity is shown at 5° C., 50° C., and 80° C. in FIGS. 9A-9C. No clear impact of pH or pH-induced polypeptide degradation on viscosity was observed at this polypeptide concentration.

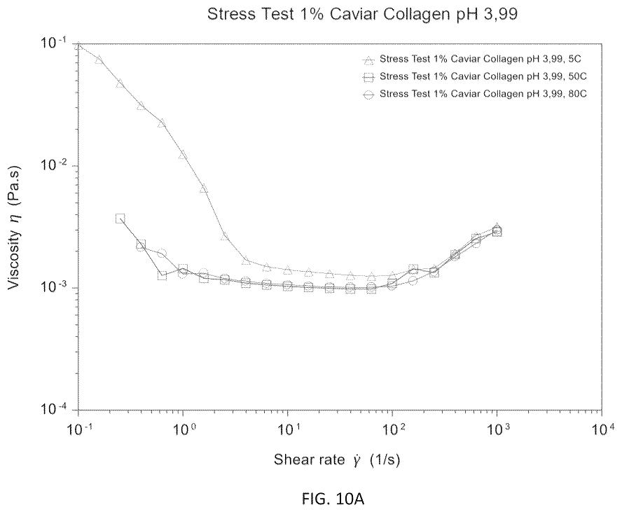

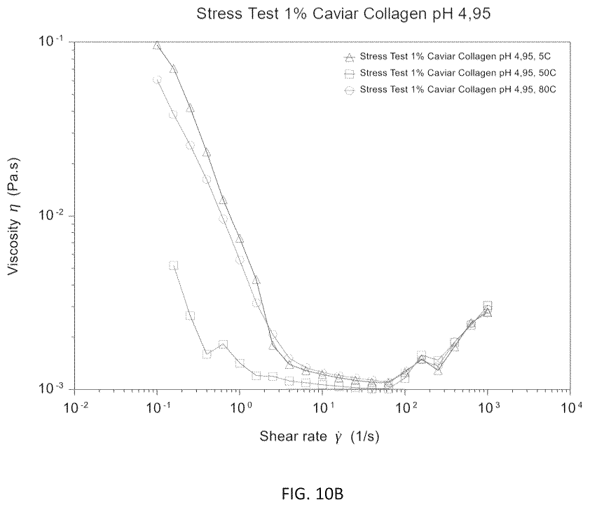

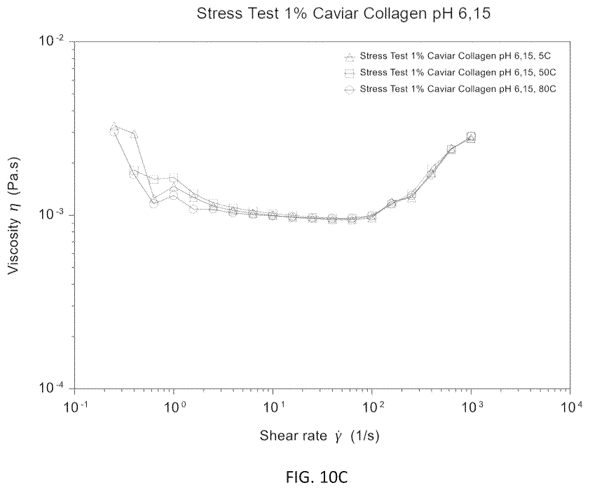

Effect of temperature on viscosity is shown at pH 3.99, 4.95, and 6.15 in FIGS. 10A-10C. No clear impact of temperature or temperature-induced polypeptide degradation on viscosity was observed at this polypeptide concentration.

### Example 7. Toxicology Analyses of a Formulation Comprising a Non-Naturally Occurring Polypeptide of the Disclosure

A variety of toxicology assays were performed in vitro to screen for any potential negative impact of formulations containing a non-naturally polypeptide having an amino acid sequence of SEQ ID NO: 8.

### 1) Bacterial Reverse Mutation Assay

The polypeptide was evaluated for the ability to induce a mutagenic response in four different strains of Salmonella typhimurium and an E. coli strain. Samples were screened at different dose levels by plating them with the tester strains both with and without Arocolor™ 1254 induced rat liver microsomes (S9). Samples are considered mutagenic if they cause an increase in revertant colonies above the spontaneous background level. The assay is known in the art and is performed compliant with OECD 4714 Guideline for Testing of Chemicals: Bacterial Reverse Mutation Assay.

A powder of a polypeptide having an amino acid sequence of SEQ ID NO: 8 was prepared in sterile deionized water at 5 concentrations: 5 mg/plate, 1 mg/plate, 0.5 mg/plate, 0.1 mg/plate, and 0.05 mg/plate. Testing was done with the appropriate solvent control and positive controls were plated with overnight cultures on selective minimal agar in the presence and absence of Aroclor-induced rate liver S9. All were plated in triplicate.

Results showed that test strains were sensitive to the positive control mutagens and showed the appropriate mutagenic response. The spontaneous reversion rate indicated that the strains were sensitive to the detection of potentially genotoxic agents. The formulations were not found to be cytotoxic to the test systems. The metabolic activation using the S9 activation mixture showed an active microsomal preparation. The formulations showed no detectable genotoxic activity at any concentration either in the presence or absence of S9 enzyme activation.

### 2) EpiDerm™ Skin Model In Vitro Toxicity Testing System

Sturgeon collagen was evaluated for irritancy potential utilizing the MatTek Corporation EpiDerm™ in vitro toxicity testing system as is known in the art. Briefly, normal, human-derived epidermal keratinocytes (NHEK) which have been cultured to form a multilayered, highly differentiated model of the human epidermis were tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (1%) was used as a positive control. Results are depicted in Table 15 (GEL-CAV-A indicating the treatment with polypeptide having an amino acid sequence of SEQ ID NO: 8).

TABLE 15
Results of in vitro toxicity testing

| Article | | Percent | Percent |
|---|---|---|---|
| (% & Exposure) | System | Viability | Inhibition |
| GEL-CAV-A EpiDerm; Lot | | | |
| Number: PP6-CAV-20-342 | | | |
| (100% - 20 hrs.) | EpiDerm | 80 | 20 |
| (100% - 4.5 hr.) | EpiDerm | 98 | 2 |
| (100% - 1 hr.) | EpiDerm | 101 | −1 |
| Triton X-100 | | | |
| (1% - 20 hrs.) | EpiDerm | 5 | 95 |
| (1% - 4.5 hr.) | EpiDerm | 94 | 6 |
| (1% - 1 hr.) | EpiDerm | 99 | 1 |

The time at which viability would be 50%, ET-50, for the polypeptide was determined to be greater than 24 hours, and the positive control at 9.4 hours. Standard ranges are shown in Table 16, according to the manufacturer.

TABLE 16
Standard ranges for EpiDerm ™ Skin
Model in vitro Toxicity Testing System

| ET-50 (hrs) | Expected In vivo Irritancy | Example |
|---|---|---|
| <0.5 | Severe, probably corrosive | Conc. Nitric Acid |
| 0.5-4 | Moderate | 1% Sodium Dodecyl Sulfate |
| 4-12 | Moderate to Mild | 1% Triton X-100 |
| 12-24 | Very Mild | Baby Shampoo |
| 24 | Non-irritating | 10% Tween 20 |

Accordingly, the polypeptide has an expected in vivo dermal irritancy potential in the non-irritating range.

### 3) EpiOcular™ Tissue Model In Vitro Toxicity Testing System

The polypeptide was evaluated for irritancy potential utilizing the MatTek Corporation EpiOcular™ in vitro toxicity testing system as is known in the art. Briefly, normal, human-derived epidermal keratinocytes which have been cultured to form a stratified, squamous epithelium similar to that found in the cornea were tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (0.3%) is used as a positive control. Results are depicted in Table 17 (GEL-CAV-A indicating the treatment with polypeptide having an amino acid sequence of SEQ ID NO: 8).

TABLE 17
Results from in vitro toxicity testing

| Article | | Percent | Percent |
|---|---|---|---|
| (% & Exposure) | System | Viability | Inhibition |
| GEL-CAV-A EpiOcular; Lot | | | |
| Number: PP6-CAV-20-342 | | | |
| (20% - 4 hrs.) | EpiOcular | 101 | −1 |
| (20% - 1 hr.) | EpiOcular | 98 | 2 |
| (20% - 20 mins.) | EpiOcular | 111 | −11 |
| Triton X-100 | | | |
| (0.3% - 1 hr.) | EpiOcular | 11 | 89 |
| (0.3% - 20 mins.) | EpiOcular | 49 | 51 |
| (0.3% - 5 mins.) | EpiOcular | 82 | 18 |

The time at which viability would be 50%, ET-50, was determined and then used to estimate the rabbit Draize eye score: Draize=−4.74+101.7/(ET-50)\hat{ }0.5 as is known in the art. The polypeptide was found to have an ET-50 greater than 256 minutes, and an estimated Draize ocular irritation score of 0 (the positive control at 19.6 minutes/Draize 18.2). Standard ranges are shown in Table 18 according to the manufacturer.

TABLE 18
Standard ranges for EpiOcular ™ Tissue
Model in vitro toxicity testing system

| | Irritancy | | EpiOcular |
|---|---|---|---|
| Draize Score | Classification | Example | ET-50 (min) |
| 0-15 | Non-irritating, | PEG-75 Lanolin, | >256-26.5 |
| | Minimal | Tween 20 | |
| 15.1-25 | Mild | 3% Sodium Dodecyl | <26.5-11.7 |
| | | Sulfate | |
| 25.1-50 | Moderate | 5% Triton X-100 | <11.7-3.45 |
| 50.1-110 | Severe, Extreme | 5% Benzalkonium | <3.45 |
| | | Chloride | |

Accordingly, the polypeptide has a non-irritating irritancy classification.

### 4) Repeated Insult Patch Study

The polypeptide was evaluated to determine its ability to sensitize the skin of volunteer subjects with normal skin using an occlusive repeated insult patch study as is known in the art. Briefly, repeated insult patch evaluation is a modified predictive patch study that can detect weak sensitizers that require multiple applications to induce a cell-mediated (Type IV) immune response sufficient to cause an allergic reaction. Irritant reactions may also be detected using this evaluation method, although this is not the primary purpose of this procedure. Sodium laurylsulfate, 0.2% aqueous solution served as a positive control.

Ninety-five (95) subjects completed the study. Under the conditions employed in this study, there was no evidence of sensitization to the polypeptide formulation.

### Example 8. Human Clinical Studies of a Formulation Comprising a Non-Naturally Occurring Polypeptide of the Disclosure

This example demonstrates an anti-aging study to assess the anti-wrinkle efficacy of formulations comprising non-naturally occurring polypeptides of the disclosure in comparison with a placebo product. Female subjects with healthy skin in the face and visible wrinkles in the periorbital regions are enrolled. Skin hydration effects are measured by Corneometer, skin elasticity and firmness effects are measured by Cutometer, and epidermal thickness is measured by Vivascope. Additionally, objective evaluation of fine lines and wrinkles, brightness and redness are performed and images are taken (Colorface) for image analysis. Assessments are performed before product application, directly after the first product application, as well as after 4 and 12 weeks of product application. Subjects fill in a questionnaire concerning product traits.

Test formulation: 0.1% (w/w) polypeptide having the amino acid sequence of SEQ ID NO: 8.

TABLE 19
Gel Cream Formulations.

| Ingredient Name | INCI Name | % wt. | Role |
|---|---|---|---|
| Water | Aqua | 77.65 (for | Solvent |
| | | actives) | |
| | | 87.75 (for | |
| | | placebo) | |
| Cosphaderm LA-T | Levulinic Acid, | 4 | Preservative |
| | Glycerin, Sodium | | |
| | Levulinate, Aqua | | |
| Tego Care 450 | Polyglyceryl-3 | 2 | Surfactant |
| | Methylglucose | | |
| | Distearate | | |
| DUB MUG | Glyceryl Undecylenate | 2 | Preservative |
| Jojobaöl | Simmondsia Chinensis | 2 | Oil phase |
| kaltgepresst kbA | (Jojoba) Seed Oil | | |
| Squalane, | Squalan | 1.25 | Oil phase |
| vegetable | | | |
| Sepimax ZEN | Polyacrylate | 1 | Thickener |
| | Crosspolymer-6 | | |
| Active Ingredient | | 0.1 | Active |
| Powder | | | |
| (polypeptide) | | | |

### Parameters:

P1: Visual Assessment: appearance of fine lines and wrinkles, brightness, redness [trained grader]
P2: Skin hydration [Corneometer®]
P3: Skin elasticity and firmness [Cutometer®]
P4: Dermal thickness [Vivascope®]
P5: Photographic Imaging, Full Face and Profiles (Left and Right), CP, Std60 [ColorFace®]
P6: Image Analysis (e.g., fine lines and wrinkles, dark spots) [Newtone]
P7: Questionnaire (up to 10 questions) [Subject]

### Test Area

The measurement of the anti-wrinkle properties (skin roughness) are performed periorbitally, i.e., in the region of crow's feet. Skin moisturizing and firming effects of the cosmetic product are evaluated on the bones of the cheeks. The study is performed in split-face design.

### Test Procedure

Day 1: Baseline measurement after acclimatization: P1-P5
Dispensing of test products to the subjects
Application of test product by subjects under supervision: P1-P5, P7
Day 2-85: Application of test product by the subjects at home, twice daily
Day 29: Measurement after acclimatization: P1-P5
Day 57: Compliance check via telephone
Day 86: Measurement after acclimatization: P1-P5, P7, return of products
After Day 86: P6

### Panel

100 female subjects (approximately 33 per product) aged between 35 and 70 years with visible eye wrinkles according to proDERM score 3 to 6 with no further specific inclusion criteria are included. It is expected that 28 subjects finish per product. Exploratory, randomized, blind for subjects, intra-individual comparison, split-face, placebo-controlled; comparison between test product and placebo per sub-group, comparison between assessment times (baseline day 1 before product application, day one after product application, after 4 and 12 weeks of product application).

### Analysis

Comparison of times per test product P1-P4
Descriptive statistics for P6, P7
Comparison of each test product vs. reference product are done separately

### Climate Conditions

All investigations are performed in rooms that are completely air-conditioned, especially equipped for the above described tests after a defined period of acclimation of the panelists. The last application usually takes place the evening before the measurements.

### Cutometer®-Skin Firmness by Measuring Total Elasticity and Elastic Recovery

Skin elasticity is measured with a Cutometer®. The measuring principle is based on a suction method. In the measuring head, a vacuum is induced that is set to 300 mbar. The skin on the measured area is sucked into the opening of the measuring head for 5 seconds with a subsequent measuring period of another 5 seconds after release. Using an optical measuring system, how far the skin is sucked into the measuring head is detected contactlessly; this value gives a measure for skin elasticity. From the resultant measuring curves, 2 parameters are calculated: Total elasticity Uf and quotient of elastic relaxation to total elasticity Ur/Uf.

### Corneometer®-Skin Hydration

Measurement of stratum corneum hydration is performed by the electrical capacitance method with the Corneometer® CM 825 (Courage & Khazaka, Cologne, Germany). The measuring principle is based on changes in the capacitance of the measuring head, functioning as a condensator. Between the conductors consisting of gold, an electrical field is built. By these means, the dielectricity of the upper skin layer is measured. Because the dielectricity varies as a function of the skin's water content, the stratum corneum hydration can be measured. An increase in Corneometer® values shows a skin-moisturizing effect.

### Questionnaire-Self Assessment

Subjects judge the test product at the end of the study in a questionnaire with up to 10 questions. The questionnaire consists of closed questions with predefined identical options to tick. If the questionnaire strongly deviates from the given structure, additional costs are charged.

### Objective Evaluation by Trained Grader

Appearance of fine lines and wrinkles, brightness, redness.

### VivaScope®-Epidermal Thickness

The VivaScope®1500 is a device for in vivo confocal scanning laser microscopy. Confocal microscopy is a technique that allows optical sectioning of turbid objects (e.g., skin). With this technique, skin can be imaged in vivo in its native state without further preparation. This method enables an in vivo mapping of the skin up to a depth of 350 μm depending on the skin type since the different microstructures within the skin cause natural variations of the refraction index and therefore provides images with contrast. For example, cytoplasm with a refraction index coming close to that of water (reflectance index 1.33) is depicted with a very low contrast. Melanin and keratin (reflectance index 1.7), however, have a relatively high refraction index and thus act as natural contrasting agents. The VivaScope® 1500 can produce in vivo section of the skin with an optical section thickness less than 5 micrometers and therefore is comparable with histological skin section. The VivaCam® macrocamera allows to capture macroimages of the test area and to correlate the confocal images with the macroimage.

### Image Analysis by Newtone

### Pigmented Spot

Parameters: Colorimetric visibility; color parameters in and outside of the spots (based on baseline spot detection); calculation of contrast Morphological visibility—contrast to the skin: conspicuous area (apparent surface detection in contrast to complexion, based on spot detection at each timepoint)

### Modalities: CP

View: Profile
Multi Pigmented spot
Parameters: Area, L*, a*, b*, in pigmented spots and outside, dE76 contrast
Modalities: CP
View: Profile
Crow's feet wrinkles
Parameters: Conspicuous length, conspicuous surface, conspicuous depth and conspicuous volume
Modalities: Standard60
View: Profile

### Nasolabial Folds

Parameters: Conspicuous length, conspicuous surface, conspicuous depth and conspicuous volume
Modalities: Standard60
View: Front face
   Index which can be Correlated to Radiance
Parameters: Saturation, luminosity, parameter correlated to radiance
Modalities: CP and Std60
View: Profile

### Under Eye Wrinkles

Parameters: Conspicuous length, conspicuous surface, conspicuous depth and conspicuous volume
Modalities: Standard60
View: Front face

### Forehead Wrinkles

Parameters: Conspicuous length, conspicuous surface, conspicuous depth and conspicuous volume
Modalities: Standard60
View: Front face

### Wrinkle Analysis by Colorface®

Crow's feet wrinkles, nasolabial folds, under eye wrinkles and forehead wrinkles

### Color/Tone Analysis by Colorface®

Pigmented spots, multi pigmented spots, index correlated to radiance, pores

### Example 9. A Formulation Comprising a Non-Naturally Occurring Polypeptide of the Disclosure Increased Skin Firmness

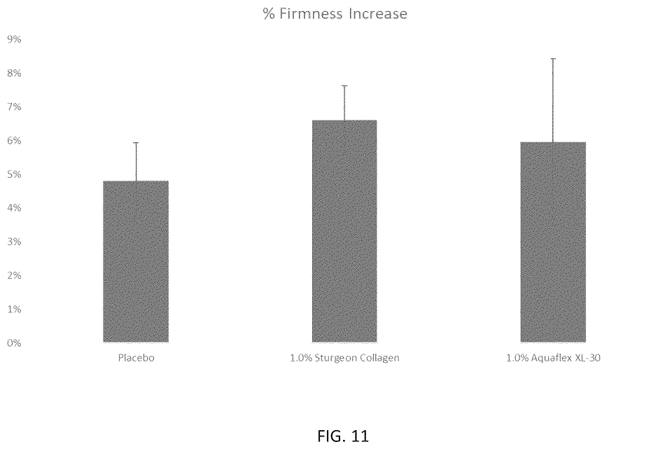

A 1% (w/w) formulation of a non-naturally occurring polypeptide having the amino acid sequence of SEQ ID NO: 8 was tested in vitro and compared to a commercially available film former that reports skin firmness benefits. The 1% (w/w) polypeptide formulation was shown to increase skin firmness approximately 6.5% as compared to placebo, and exceeded the comparable commercially available film former as shown in FIG. 11.

Placebo: 98.5% (w/w) water, 0.5% (w/w) hydroxypropyl guar, 1% (w/w) phenoxyethanol, pH 7 with 2% NaOH solution

1% (w/w) polypeptide formulation: 97.5% (w/w) water, 1% (w/w) polypeptide having an amino acid sequence of SEQ ID NO: 8, 0.5% (w/w) hydroxypropyl guar, 1% (w/w) phenoxyethanol, pH 7 with 2% NaOH solution

1% (w/w) commercially available film former: 97.5% (w/w) water, 1% (w/w) Aquaflex XL-30 (Ashland), 0.5% (w/w) hydroxypropyl guar, 1% (w/w) phenoxyethanol, pH 7 with 2% Citric Acid solution

Test substrate: Bioskin resiliency model (hardness 0.18 S)—Beaulax, Japan

Product application: 2 mg/cm; Drying time: 10 minutes

With a texture analyzer (TA XT Plus Connect, Stable Micro Systems), using a spherical probe (d: ½″), the substrate was compressed to a strain value of 20%. The maximum force necessary to apply the target strain was measured. The product was then applied to substrate (2 mg/cm²) and allowed to dry for 10 minutes. The measurement was repeated and the new force value was recorded. The force variation relative to the initial force was reported using the formula:

$$\% Firmness Increase = \frac{( {Force}_{test product} - {Force}_{substrate} )}{{Force}_{substrate}}$$

### Example 10. In Vitro Studies on Skin Cells

Healthy skin is primarily composed of collagen types I and III, hyaluronans, fibronectin and elastin, and a basal lamina that includes other proteins such as laminins and collagen IV. Fibroblasts are the major cell type that produces these structural proteins, including collagen. Collectively the proteins are known as extra cellular matrix (ECM) and they support the skin's structure. Fibroblast output of collagen decreases with age, so fibroblasts are a primary target for the activity of cosmetics to try to rescue skin aging.

Keratinocytes are the major cell type forming the epidermis, or outer layers of the skin. HaCaT cells are an immortal keratinocyte cell line derived from adult skin. Both cell types were used to demonstrate the benefits of a non-naturally occurring polypeptide having an amino acid sequence of SEQ ID NO: 8 on skin (indicated as “Cav” in figures). These cells have a high turnover and receive the brunt of everyday pollution and radiation. They are negatively affected by the environments they are subjected to, which leads to increased inflammation and damage to our natural skin barrier. Hallmarks used to assess keratinocyte health include inflammatory markers, cell turnover, and DNA integrity.

### 1) A Non-Naturally Occurring Polypeptide Having an Amino Acid Sequence of SEQ ID NO: 8 is Non-Toxic to Human Fibroblasts and Keratinocytes

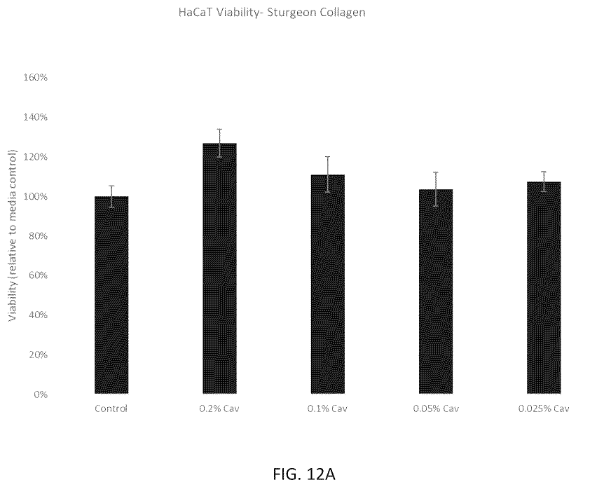

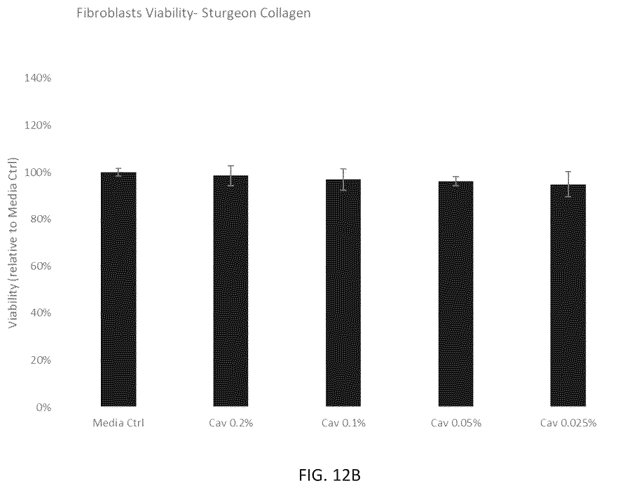

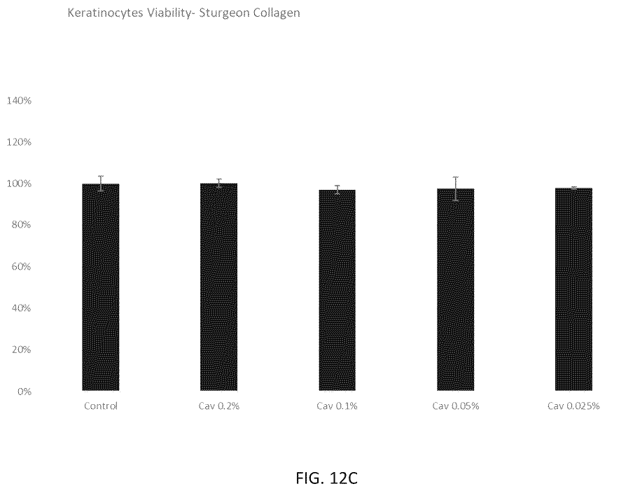

Human primary fibroblasts, HaCaT cells, and human primary keratinocytes treated with a non-naturally occurring polypeptide having an amino acid sequence of SEQ ID NO: 8 in vitro showed no sign of toxicity, as shown in FIGS. 12A-12C, indicating the product is safe as a topical ingredient at the dosages tested.

### Protocol:

The cells were seeded at confluency in a 96-well plate. 24 hours later, the media was changed to low serum media (to avoid any effects due to serum). The cells were treated with a non-naturally occurring polypeptide having the amino acid sequence of SEQ ID NO: 8 in the same low serum media for 24 hours. Post treatment with the polypeptide, the supernatants were saved, and cells were incubated with MTT dye for 60 minutes at 37° C. MTT is metabolized to formazan salts by viable cells. These salts were dissolved using isopropanol and the color produced was quantified using a cell plate reader.

### 2) A Non-Naturally Occurring Polypeptide Having an Amino Acid Sequence of SEQ ID NO: 8 Promotes Keratinocyte Growth and Regeneration

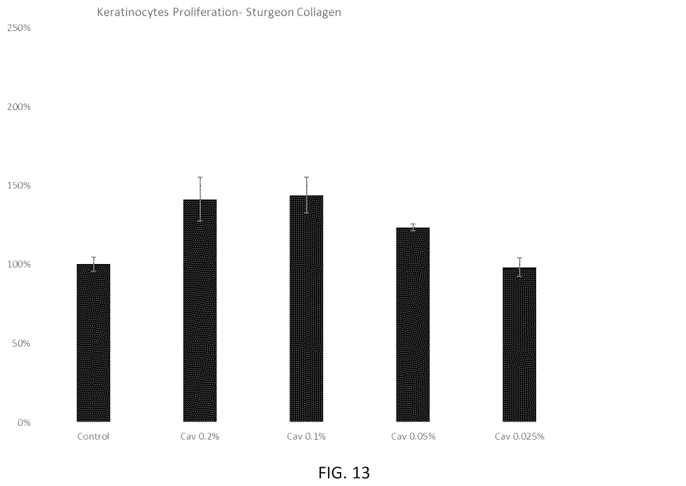

Keratinocytes treated with a non-naturally occurring polypeptide having an amino acid sequence of SEQ ID NO: 8 showed a dose-dependent increase in keratinocyte growth and regeneration. Similar results were seen immortal HaCaT keratinocytes. As shown in FIG. 13 (human primary keratinocytes), the polypeptide demonstrated a dose-dependent stimulation of cellular growth and regeneration in keratinocytes, with a 40% increase in cell numbers at 0.2% (w/w) and 0.1% (w/w) treatment, when compared with control cells.

### 3) A Non-Naturally Occurring Polypeptide Having an Amino Acid Sequence of SEQ ID NO: 8 Stimulates Collagen Production

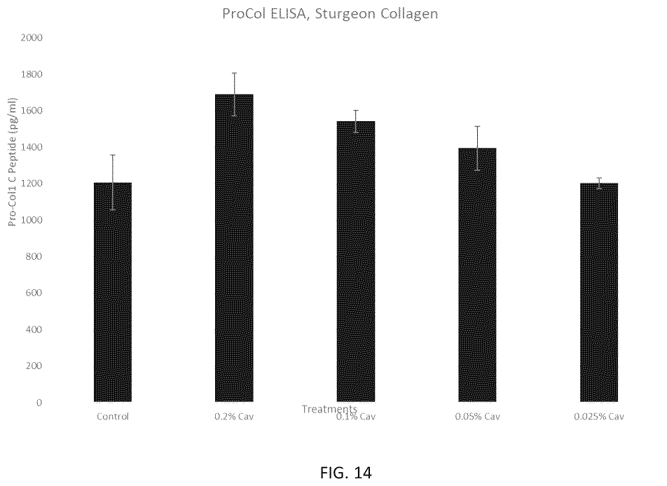

A non-naturally occurring polypeptide having an amino acid sequence of SEQ ID NO: 8 stimulated production of collagen I production by in vitro fibroblasts as shown in FIG. 14.

ELISA Protocol: Primary human fibroblasts were cultured in standard media DMEM/F12+10% FBS. Supernatants were used to determine the level of collagen type I present. The kit used was Takara Procollagen type I C-peptide detection ELISA kit. Manufacturer's protocol was followed to measure the quantity of collagen type I in the supernatants.

In a second method of analysis, microarray data reporting the levels of RNA for a variety of human collagens showed a 2.5-3-fold increase in expression of these collagens in fibroblasts treated with the polypeptide. Table 20 depicts the microarray data.

Microarray RNA analysis Protocol: The cells were seeded at confluency in 6-well plates. 24 hours later the media was changed to low serum media. The cells were treated with 0.05% (w/w) of the polypeptide and control. The QIAGEN RNeasy kit was used to extract the RNA and the extracted RNA for analysis.

TABLE 20
Microarray data

| | 7 Avg | 8 Avg | Fold | | FDR | Gene | | |
|---|---|---|---|---|---|---|---|---|
| ID count: 10 | (log2) | (log2) | Chan . . . | P-val | P-val | Symbol | Description | Group |
| TC1700011088.h . . . | 9.08 | 7.42 | 3.16 | 4.15E−06 | 0.0011 | COL1A1 | collagen, type I, alpha 1 | Multiple . . . |
| TC0700008358.h . . . | 15.3 | 13.86 | 2.71 | 7.01E−06 | 0.0011 | COL1A2 | collagen, type I, alpha 2 | Multiple . . . |
| TC0200016193.h . . . | 14.16 | 12.26 | 3.73 | 7.69E−06 | 0.0011 | COL6A3 | collagen, type VI, alpha 3 | Multiple . . . |
| TC0600012314.h . . . | 11.16 | 9.46 | 3.26 | 8.77E−06 | 0.0011 | COL12A1 | collagen, type XII, alpha 1 | Multiple . . . |
| TC2100007446.h . . . | 13.82 | 12.32 | 2.82 | 1.01E−05 | 0.0012 | COL6A1 | collagen, type VI, alpha 1 | Multiple . . . |
| TC0200010239.h . . . | 10.42 | 8.93 | 2.81 | 2.99E−05 | 0.0015 | COL3A1; . . . | collagen, type III, alpha 1; . . . | Multiple . . . |
| TC2100007451.h . . . | 9.11 | 7.91 | 2.3 | 9.13E−05 | 0.0022 | COL6A2 | collagen, type VI, alpha 2 | Multiple . . . |
| TC0500011163.h . . . | 10.42 | 9.22 | 2.29 | 0.0001 | 0.0023 | COL4A3BP | collagen, type IV, alpha 3 . . . | Multiple . . . |
| TC0900009127.h . . . | 8.32 | 6.39 | 3.8 | 0.0004 | 0.0052 | COL5A1 | collagen, type V, alpha 1 | Multiple . . . |
| TC1300008010h . . . | 5.98 | 4.98 | 2 | 0.0022 | 0.0155 | COL4A2 | collagen, type IV, alpha 2 | Multiple . . . |

In addition to the upregulation of collagens, the polypeptide was found to increase the levels of RNA for a variety of genes involved in several pathways responsible for proliferation, migration, and adhesion.

### Upregulated Pathways:

### VEGFA-VEGFR2 Signaling Pathway

Number of upregulated genes: 74
Number of down regulated genes: 12
Significance: 7.74

TABLE 21
Exemplary upregulated genes in the
VEGFA-VEGFR2 signaling pathway

| Symbol | Fold change | P-value | Description |
|---|---|---|---|
| MYOC1 | 3.25 | 9.25E−05 | Myosin IC |
| FLII | 4.08 | 1.06E−05 | Flightless I actin |
| | | | binding protein |
| ROCK1 | 3.72 | 8.22E−07 | Rho-associated, coiled- |
| | | | coil containing protein |
| | | | kinase 1 |
| ROCK2 | 5.03 | 6.31E−05 | Rho-associated, coiled- |
| | | | coil containing protein |
| | | | kinase 2 |
| CLTC | 2.7 | 7.06E−06 | Clathrin, heavy |
| | | | chain (Hc) |
| LIMK 1 | 4.24 | 2.04E−06 | Serine/threonine |
| | | | Kinase |
| EGR1 | 3.28 | 2.27E−05 | Early growth |
| | | | response 1 |

### Focal Adhesion Pathway

No of upregulated genes: 53
No of down regulated genes: 0
Significance: 9.93

TABLE 22
Exemplary upregulated genes in the Focal Adhesion Pathway

| Symbol | Fold change | P-value | Description |
|---|---|---|---|
| ITGA3 | 3.45 | 5.71E−07 | Integrin alpha 3 |
| TNC | 3.13 | 5.53E−05 | Tenascin |
| LAMC1 | 3.15 | 1.15E−05 | Lamini, gamma 1 |
| FLNA | 6.5 | 1.47E−07 | Actin-binding protein |
| | | | that regulates |
| | | | reorganization of |
| | | | actin cytoskeleton |
| TLN1 | 3.8 | 0.0001 | Cytoskeletal protein |
| ZYX | 3.8 | 6.81E−06 | Zinc-binding |
| | | | phosphoprotein and |
| | | | is found in mature |
| | | | adhesions |
| DIAPH1 | 3.78 | 8.79E−05 | Regulates cell |
| | | | morphology and |
| | | | cytoskeletal |
| | | | reorganization |

### Endothelin Pathway

No of upregulated genes: 48
No of down regulated genes: 4
Significance: 3.57

TABLE 23
Exemplary upregulated genes in the Endothelin Pathway

| Symbol | Fold change | P-value | Description |
|---|---|---|---|
| TRIOBP | 3.91 | 4.55E−06 | TRIO and F-actin |
| | | | binding protein; |
| | | | nucleolar protein 12 |
| WNK1 | 3.11 | 9.10E−05 | Serine/threonine |
| | | | protein kinase |
| MMP2 | 3.08 | 1.13E−06 | Matrix |
| | | | metallopeptidase 2 |
| VCAN | 3.31 | 3.10E−06 | Versican |
| ACTA2 | 2.52 | 0.0002 | Actin, alpha 2 |
| GNA12 | 3.88 | 5.99E−06 | Guanine nucleotide |
| | | | binding protein (G- |
| | | | protein) alpha 12 |
| EGR1 | 3.28 | 2.27E−05 | Early growth |
| | | | response 1 |

### EGF/EGER Signaling Pathway

No of upregulated genes: 32
No of down regulated genes: 4
Significance: 4.83

TABLE 24
Exemplary upregulated genes in the EGF/EGFR Signaling Pathway

| Symbol | Fold change | P-value | Description |
|---|---|---|---|
| ATXN2 | 3.6 | 2.48E−05 | Interact with |
| | | | endoplasmic |
| | | | reticulum |
| JAK1 | 3.15 | 1.88E−05 | Involved in cell |
| | | | growth, survival, |
| | | | development, and |
| | | | differentiation |
| RPS6KA2 | 3.12 | 1.96E−06 | Ribosomal protein |
| ROCK1 | 3.72 | 8.22E−07 | Rho-associated, |
| | | | coiled-coil |
| | | | containing protein |
| | | | kinase 1 |
| SHC1 | 2.83 | 2.57E−06 | Regulation of |
| | | | apoptosis |
| IQGAP1 | 2.65 | 1.94E−05 | Scaffold protein, |
| | | | help in modulating |
| | | | several cellular |
| | | | activities. |
| PLCG1 | 2.59 | 0.0011 | Ribosomal protein |

### TGF-Beta Signaling Pathway

No of upregulated genes: 33
No of down regulated genes: 3
Significance: 6.92

TABLE 25
Exemplary upregulated genes in the TGF-beta signaling pathway

| Symbol | Fold change | P-value | Description |
|---|---|---|---|
| SMURF1 | 2.68 | 4.20E−06 | SMAD specific E3 |
| | | | ubiquitin protein |
| | | | kinase 1 |
| SPTBN1 | 3.41 | 4.83E−06 | Responsible for |
| | | | cellular shape, |
| | | | protection of |
| | | | membranes against |
| | | | stress |
| PAK2 | 2.71 | 1.68E−05 | Stimulates cell |
| | | | survival and cell |
| | | | growth |
| ROCK1 | 3.72 | 8.22E−07 | Rho-associated, |
| | | | coiled-coil |
| | | | containing protein |
| | | | kinase 1 |
| SHC1 | 2.83 | 2.57E−06 | Regulation of |
| | | | apoptosis |
| TGFBR3 | 2.6 | 2.05E−05 | Transforming |
| | | | growth factor beta |
| | | | receptor III |
| TGFBR1 | 2.6 | 0.0062 | Transforming |
| | | | growth factor beta |
| | | | receptor I |

### 4) A Non-Naturally Occurring Polypeptide Having an Amino Acid Sequence of SEQ ID NO: 8 Promotes Wound Healing Activity

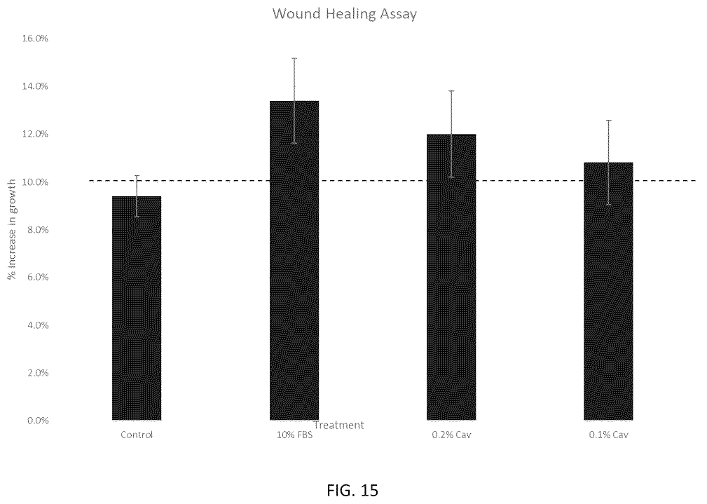

Wound healing is a dynamic process that includes a sequence of events, including cell proliferation and migration. Fibroblast migration and proliferation play a crucial role in wound closure by secreting various chemicals, including collagen and other matrix proteins. Treatment of in vitro human dermal fibroblasts with a non-naturally occurring polypeptide having an amino acid sequence of SEQ ID NO: 8 showed wound healing activity in an in vitro wound-healing model as shown in FIG. 15, as cells proliferated and closed a gap induced by scratching a confluent layer of fibroblasts. Additionally, microarray data was consistent with the polypeptide having a wound healing benefit. The data also showed upregulation of genes involved in several pathways responsible for cell proliferation, migration, and adhesion.

Protocol: The cells were seeded at confluency in 24 well plate. 24 hours later the media was changed to low serum media and the cells were starved for 6-8 hours. Post starvation, the wells containing cells were scratched and treated. Images were taken at this time (time 0 hours) and after 24 hours. Images were analyzed using Image J software.

### 5) A Non-Naturally Occurring Polypeptide Having an Amino Acid Sequence of SEQ ID NO: 8 Increases Cell Viability of Keratinocytes Exposed to Urban Dust Pollution

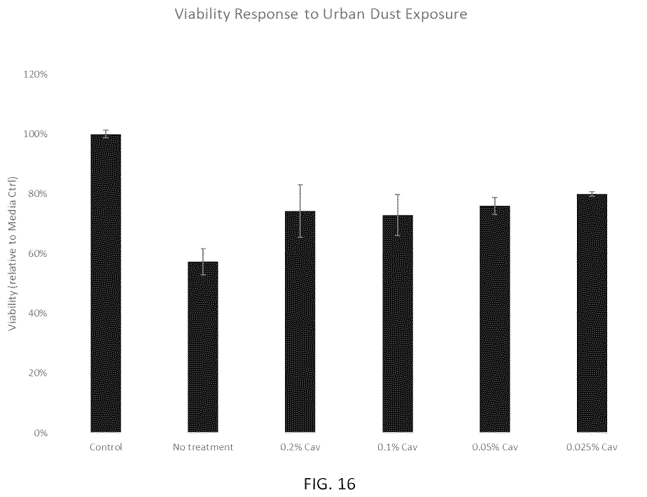

Pre-treatment of HaCaT cells with a non-naturally occurring polypeptide having an amino acid sequence of SEQ ID NO: 8 prior to exposure to a government-certified urban dust sample protected the cells. Cell viability was approximately 20% higher with pre-treatment of the polypeptide when compared to no polypeptide pre-treatment (control being no pretreatment, no urban dust exposure) as shown in FIG. 16.

Urban dust concentration used: 2 mg/ml.

Protocol: The cells were seeded at confluency in 96-well plate. The cells were treated with the polypeptide for 24 hours (pre-treating the cells before they were exposed to Urban dust). The desired urban dust concentration was prepared, and the cells were exposed to it for 24 hours. Post urban dust exposure, the supernatants were stored to run different inflammatory cytokines and the cells were incubated with MTT dye for 60 minutes at 37° C. MTT is metabolized to formazan salts by viable cells. These salts were dissolved using isopropanol and the color produced was quantified using a cell plate reader.

### 6) A Non-Naturally Occurring Polypeptide Having an Amino Acid Sequence of SEQ ID NO: 8 has Antioxidative Capacity

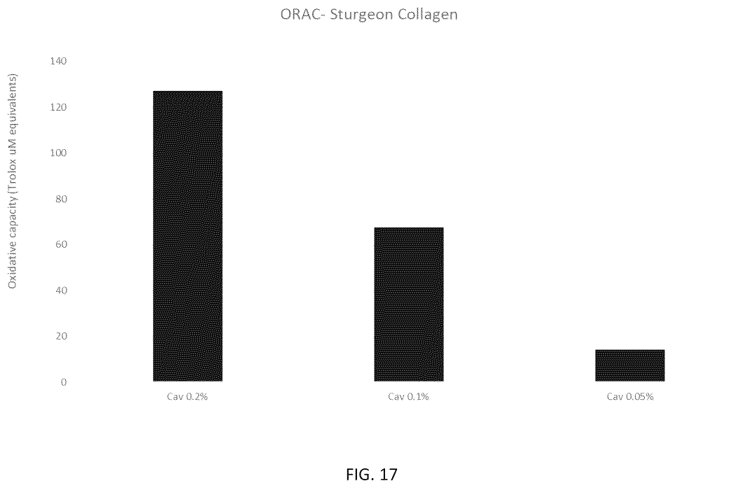

The oxygen radical absorbance capacity (ORAC) assay (a cell-free assay that uses a fluorescent readout) was used to show the antioxidant capacity of sturgeon collagen. Data was reported in Trolox (Vitamin E) equivalents. In the form of a 0.2% (w/w) solution, a non-naturally occurring polypeptide having an amino acid sequence of SEQ ID NO: 8 showed anti-oxidative properties equivalent to 130 μM Trolox as shown in FIG. 17.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

1. A cosmetic formulation comprising:
a polypeptide comprising an amino acid sequence having at least 80% sequence identity to SEQ ID NO: 32, or a polypeptide comprising an amino acid sequence having at least 80% sequence identity to a truncate of SEQ ID NO: 32,
wherein the cosmetic formulation is selected from the group consisting of a cream, a gel, a gel cream, an oil, an ointment, a serum, a foam, a lotion, a paste, a balm, a solution, a suspension, and a powder.

2. (canceled)

3. The cosmetic formulation of claim 1, further comprising:
one or more additional ingredients selected from the group consisting of: levulinic acid, polyglyceryl-3 methylglucose distearate, glyceryl undecylenate, Simmondsia chinensis (Jojoba) seed oil, polyacrylate cross-polymer, squalane, sodium hyaluronate, acrylic acid polymers (carbomers), pentylene glycol, sodium lauryl sulfoacetate, sodium oleoyl sarcosinate, sodium oleate, Ricinus communis (castor) seed oil, Copernicia cerifera (Carnauba) wax, Candelilla wax, Theobroma cacao (Cocoa) Seed Butter, isononyl isononanoate, ozokerite, isopropyl titanium triisostearate, polyhydroxystearic acid, iron oxide, titanium dioxide, sodium levulinate, and hydroxypropyl guar.

4. The cosmetic formulation of claim 1, wherein:
the polypeptide comprises an amino acid sequence having at least 85% sequence identity to SEQ ID NO: 32, or the polypeptide comprises an amino acid sequence having at least 85% sequence identity to a truncate of SEQ ID NO: 32;
the polypeptide comprises an amino acid sequence having at least 90% sequence identity to SEQ ID NO: 32, or the polypeptide comprises an amino acid sequence having at least 90% sequence identity to a truncate of SEQ ID NO: 32;
the polypeptide comprises an amino acid sequence having at least 95% sequence identity to SEQ ID NO: 32, or the polypeptide comprises an amino acid sequence having at least 95% sequence identity to a truncate of SEQ ID NO: 32;
the polypeptide comprises an amino acid sequence having at least 98% sequence identity to SEQ ID NO: 32, or the polypeptide comprises an amino acid sequence having at least 98% sequence identity to a truncate of SEQ ID NO: 32; or
the polypeptide comprises an amino acid sequence having 100% sequence identity to SEQ ID NO: 32, or the polypeptide comprises an amino acid sequence having 100% sequence identity to a truncate of SEQ ID NO: 32.

5-8. (canceled)

9. The cosmetic formulation of claim 1, wherein the truncate of SEQ ID NO: 32 comprises an N-terminal truncation, a C-terminal truncation, or both, relative to SEQ ID NO: 32.

10. The cosmetic formulation of claim 9, wherein the N-terminal truncation is an N-terminal truncation of 50 amino acids to 750 amino acids relative to SEQ ID NO: 32; and/or wherein the C-terminal truncation is a C-terminal truncation of 50 amino acids to 600 amino acids relative to SEQ ID NO: 32.

11. (canceled)

12. The cosmetic formulation of claim 1, wherein the polypeptide comprises or consists of an amino acid sequence having at least 80%, at least 90%, at least 95%, or at least 98% sequence identity to SEQ ID NO: 8.

13-18. (canceled)

19. The cosmetic formulation of claim 1, wherein the polypeptide is 50 amino acids to 250 amino acids in length.

20. The cosmetic formulation of claim 1, wherein the polypeptide does not comprise one or more of: a laminin G domain, a Von Willebrand factor type A (vWA) domain, or a fibrillar collagen C-terminal domain.

21. The cosmetic formulation of claim 1, wherein the polypeptide comprises one or more collagen triple helix repeats.

22. The cosmetic formulation of claim 1, wherein the polypeptide is monomeric.

23-25. (canceled)

26. The cosmetic formulation of claim 1, wherein fewer than 10% of prolines present in the polypeptide are hydroxylated.

27. The cosmetic formulation of claim 1, wherein the polypeptide is non-hydroxylated.

28. (canceled)

29. The cosmetic formulation of claim 1, wherein the polypeptide comprises less than 5 wt. % glycosylation.

30. The cosmetic formulation of claim 1, wherein the polypeptide is present in the cosmetic formulation at an amount of 0.001% to 30% w/w.

31-32. (canceled)

33. The cosmetic formulation of claim 1, wherein the cosmetic formulation further comprises a topical carrier selected from the group consisting of: a liposome, a biodegradable microcapsule, a lotion, a spray, an aerosol, a dusting powder, a biodegradable polymer, mineral oil, triglyceride oil, silicone oil, glycerin, glycerin monostearate, an alcohol, an emulsifying agent, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, water, digylcerol, and/or a fatty acid.

34. (canceled)

35. The cosmetic formulation of claim 1, further comprising a preservative selected from the group consisting of: tocopherol, diiodomethyl-p-tolylsulfone, 2-bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-ethylbicyclooxazolidine, phenoxyethanol, butylene glycol, 1,2 hexanediol, methyl paraben, sorbic acid, Germaben® II, rosemary extract, ethylenediaminetetraacetic acid (EDTA), benzoic acid or salts thereof, and/or chlorhexidine.

36. (canceled)

37. A personal care product comprising the cosmetic formulation of claim 1, wherein the personal care product is selected from the group consisting of: a mask, a skin cleaners, a cleansing cream, a cleansing lotion, a facial lotion, a body lotion, a shower gel, an antiperspirant, a deodorant, a shave cream, a depilatory, a face oil, a lip oil, a body oil, a facial cleanser, a cleansing milk, a cleansing pad, a facial wash, a facial cream, a body cream, a facial moisturizer, a body moisturizer, a facial serum, a facial mask, a body mask, a facial toner, a facial mist, an eye cream, an eye serum, an exfoliator formula, a lip balm, a lipstick, a hair shampoo, a hair conditioner, a body shampoo, a hair serum, a scalp serum, a hair mist, a hair spray, a foundation, a tinted multifunctional cream, an eye shadow, a concealer, a mascara, and/or any combination thereof.

38. (canceled)

39. A method comprising: applying the cosmetic formulation of claim 1 or the personal care product of claim 37 to the skin of an individual, thereby promoting, improving, and/or maintaining youthful skin of the individual.

40. The method of claim 39, wherein promoting, improving, and/or maintaining youthful skin of the individual comprises improving firmness of the skin of the individual, wherein improving firmness of the skin of the individual comprises increasing skin firmness by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on measuring the resistance of the skin to negative pressure.

41. (canceled)

42. The method of claim 39, wherein promoting, improving, and/or maintaining youthful skin of the individual comprises improving elasticity of the skin of the individual, wherein improving elasticity of the skin of the individual comprises increasing skin elasticity by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on measuring the ability of the skin to return to its original position after deformation.

43. (canceled)

44. The method of claim 39, wherein promoting, improving, and/or maintaining youthful skin of the individual comprises improving brightness of the skin of the individual, wherein improving brightness of the skin of the individual comprises increasing brightness of the skin by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%.

45. (canceled)

46. The method of claim 39, wherein promoting, improving, and/or maintaining youthful skin of the individual comprises improving hydration of the skin of the individual, wherein improving hydration of the skin of the individual comprises increasing skin hydration by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75% based on capacitance measurement of the skin.

47. (canceled)

48. The method of claim 39, wherein promoting, improving, and/or maintaining youthful skin of the individual comprises at least one of: improving tactile texture of the skin of the individual; improving collagen content of the skin of the individual; improving elastin content of the skin of the individual; improving redness of the skin of the individual;
improving visual texture of the skin of the individual; improving fine lines and/or wrinkles of the skin of the individual; and/or improving epidermal thickness of the skin of the individual.

49-57. (canceled)

58. The method of claim 39, wherein, after the application of the cosmetic formulation, keratinocyte growth and/or regeneration in the skin is increased;
collagen production is increased; fibroblast migration, proliferation, and/or adhesion is increased; and/or keratinocyte viability after exposure to urban dust is increased.

59-67. (canceled)
